SEQUENCE LISTING (218 sequences)

SEQ ID NO: 1 - protein, 304 aa, Homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 2 - protein, 216 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 3 - protein, 120 aa, Mus musculus
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 4 - protein, 107 aa, Mus musculus
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 5 - protein, 122 aa, Mus musculus
EVQLQESGPGLVKPSQSLSLTCTVTGYSITSDYAWNWIRQFPGNKLEWMGYITYSGSTSYNPSLESRISITRDTSTNQFFLQLNSVTTEDTATYYCARGGYYGSSWGVFAYWGQGTLVTVSA

SEQ ID NO: 6 - protein, 107 aa, Mus musculus
DIQMTQSPASLSASVGETVTITCRVSENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 7 - protein, 116 aa, Mus musculus
EVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSS

SEQ ID NO: 8 - protein, 107 aa, Mus musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 9 - protein, 117 aa, Mus musculus
QVQLQQSAVELARPGASVKMSCKASGYTFTSFTMHWVKQRPGQGLEWIGYINPSSGYTEYNQKFKDKTTLTADKSSSTAYMQLDSLTSDDSAVYYCVRGSSRGFDYWGQGTLVTVSA

SEQ ID NO: 10 - protein, 107 aa, Mus musculus
DIQMIQSPASLSVSVGETVTITCRASENIYSNLAWFQQKQGKSPQLLVYAATNLADGVPSRFSGSGSGTQYSLKINSLQSEDFGIYYCQHFWGTPRTFGGGTKLEIK

SEQ ID NO: 11 - protein, 115 aa, Mus musculus
QVQLQQPGSVLVRPGASVKLSCKASGYTFTSSWMHWAKQRPGQGLEWIGHIHPNSGISNYNEKFKGKATLTVDTSSSTAYVDLSSLTSEDSAVYYCARGGRFDDWGAGTTVTVSS

SEQ ID NO: 12 - protein, 107 aa, Mus musculus
DIQMTQSPSSLSASLGERVSLTCRASQDIGSSLNWLQQEPDGTIKRLIYATSSLDSGVPKRFSGSRSGSDYSLTISSLESEDFVDYYCLQYASSPWTFGGGTKLEIK

SEQ ID NO: 13 - protein, 120 aa, Mus musculus
DVQLQESGPGLVKPSQSLSLTCTVTGYSITSDYAWNWIRQFPGNKLEWMGYITYSGSTNYNPSLKSRISITRDTSKNQFFLQLNSVTTEDTATYYCARCWDYALYAMDCWGQGTSVTVSS

SEQ ID NO: 14 - protein, 107 aa, Mus musculus
DIQMTQSPASLSASVGETVTITCRTSENIYSYLAWCQQKQGKSPQLLVYNAKTLAEGVPSRFSGSGSGTHFSLKINSLQPEDFGIYYCQHHYDTPLTFGAGTKLELK

SEQ ID NO: 15 - protein, 5 aa, Mus musculus
DYVIN

SEQ ID NO: 16 - protein, 7 aa, Mus musculus
GYTFTDY

SEQ ID NO: 17 - protein, 8 aa, Mus musculus
GYTFTDYV

SEQ ID NO: 18 - protein, 17 aa, Mus musculus
EIYPGSGTNYYNEKFKA

SEQ ID NO: 19 - protein, 4 aa, Mus musculus
PGSG

SEQ ID NO: 20 - protein, 16 aa, Mus musculus
GYTFTDYVIYPGSGTN

SEQ ID NO: 21 - protein, 11 aa, Mus musculus
RGRYGLYAMDY

SEQ ID NO: 22 - protein, 9 aa, Mus musculus
GRYGLYAMD

SEQ ID NO: 23 - protein, 13 aa, Mus musculus
ARRGRYGLYAMDY

SEQ ID NO: 24 - protein, 11 aa, Mus musculus
RASQDISNYLN

SEQ ID NO: 25 - protein, 7 aa, Mus musculus
SQDISNY

SEQ ID NO: 26 - protein, 6 aa, Mus musculus
QDISNY

SEQ ID NO: 27 - protein, 7 aa, Mus musculus
YTSRLHS

SEQ ID NO: 28 - protein, 9 aa, Mus musculus
QQGNTRPWT

SEQ ID NO: 29 - protein, 9 aa, Mus musculus
YTSGNTRPW

SEQ ID NO: 30 - protein, 12 aa, Mus musculus
YTSQQGNTRPWT

SEQ ID NO: 31 - protein, 6 aa, Mus musculus
SDYAWN

SEQ ID NO: 32 - protein, 8 aa, Mus musculus
GYSITSDY

SEQ ID NO: 33 - protein, 9 aa, Mus musculus
GYSITSDYA

SEQ ID NO: 34 - protein, 16 aa, Mus musculus
YITYSGSTSYNPSLES

SEQ ID NO: 35 - protein, 7 aa, Mus musculus
ITYSGST

SEQ ID NO: 36 - protein, 13 aa, Mus musculus
GGYYGSSWGVFAY

SEQ ID NO: 37 - protein, 11 aa, Mus musculus
GYYGSSWGVFA

SEQ ID NO: 38 - protein, 15 aa, Mus musculus
ARGGYYGSSWGVFAY

SEQ ID NO: 39 - protein, 11 aa, Mus musculus
RVSENIYSYLA

SEQ ID NO: 40 - protein, 7 aa, Mus musculus
SENIYSY

SEQ ID NO: 41 - protein, 6 aa, Mus musculus
ENIYSY

SEQ ID NO: 42 - protein, 7 aa, Mus musculus
NAKTLAE

SEQ ID NO: 43 - protein, 9 aa, Mus musculus
QHHYGTPWT

SEQ ID NO: 44 - protein, 6 aa, Mus musculus
HYGTPW

SEQ ID NO: 45 - protein, 9 aa, Mus musculus
QHHYGTPWT

SEQ ID NO: 46 - protein, 5 aa, Mus musculus
EYTMH

SEQ ID NO: 47 - protein, 7 aa, Mus musculus
GYTFTEY

SEQ ID NO: 48 - protein, 8 aa, Mus musculus
GYTFTEYT

SEQ ID NO: 49 - protein, 17 aa, Mus musculus
GISPNIGGTSYNQKFKG

SEQ ID NO: 50 - protein, 8 aa, Mus musculus
ISPNIGGT

SEQ ID NO: 51 - protein, 7 aa, Mus musculus
RGGSFDY

SEQ ID NO: 52 - protein, 5 aa, Mus musculus
GGSFD

SEQ ID NO: 53 - protein, 9 aa, Mus musculus
ARRGGSFDY

SEQ ID NO: 54 - protein, 11 aa, Mus musculus
RASQSISDYLH

SEQ ID NO: 55 - protein, 7 aa, Mus musculus
SQSISDY

SEQ ID NO: 56 - protein, 6 aa, Mus musculus
QSISDY

SEQ ID NO: 57 - protein, 7 aa, Mus musculus
YASQSIS

SEQ ID NO: 58 - protein, 9 aa, Mus musculus
QNGHSFPLT

SEQ ID NO: 59 - protein, 6 aa, Mus musculus
GHSFPL

SEQ ID NO: 60 - protein, 5 aa, Mus musculus
SFTMH

SEQ ID NO: 61 - protein, 7 aa, Mus musculus
GYTFTSF

SEQ ID NO: 62 - protein, 8 aa, Mus musculus
GYTFTSFT

SEQ ID NO: 63 - protein, 17 aa, Mus musculus
YINPSSGYTEYNQKFKD

SEQ ID NO: 64 - protein, 8 aa, Mus musculus
INPSSGYT

SEQ ID NO: 65 - protein, 8 aa, Mus musculus
GSSRGFDY

SEQ ID NO: 66 - protein, 6 aa, Mus musculus
SSRGFD

SEQ ID NO: 67 - protein, 10 aa, Mus musculus
VRGSSRGFDY

SEQ ID NO: 68 - protein, 11 aa, Mus musculus
RASENIYSNLA

SEQ ID NO: 69 - protein, 6 aa, Mus musculus
ENIYSN

SEQ ID NO: 70 - protein, 7 aa, Mus musculus
AATNLAD

SEQ ID NO: 71 - protein, 9 aa, Mus musculus
QHFWGTPRT

SEQ ID NO: 72 - protein, 6 aa, Mus musculus
FWGTPR

SEQ ID NO: 73 - protein, 5 aa, Mus musculus
SSWMH

SEQ ID NO: 74 - protein, 7 aa, Mus musculus
GYTFTSS

SEQ ID NO: 75 - protein, 8 aa, Mus musculus
GYTFTSSW

SEQ ID NO: 76 - protein, 17 aa, Mus musculus
HIHPNSGISNYNEKFKG

SEQ ID NO: 77 - protein, 8 aa, Mus musculus
IHPNSGIS

SEQ ID NO: 78 - protein, 6 aa, Mus musculus
GGRFDD

SEQ ID NO: 79 - protein, 8 aa, Mus musculus
ARGGRFDD

SEQ ID NO: 80 - protein, 11 aa, Mus musculus
RASQSISDYLH

SEQ ID NO: 81 - protein, 11 aa, Mus musculus
GRFDSQSISDY

SEQ ID NO: 82 - protein, 10 aa, Mus musculus
QNGHSFLMYT

SEQ ID NO: 83 - protein, 7 aa, Mus musculus
GHSFLMY

SEQ ID NO: 84 - protein, 13 aa, Mus musculus
YASQNGHSFLMYT

SEQ ID NO: 85 - protein, 6 aa, Mus musculus
SDYAWN

SEQ ID NO: 86 - protein, 8 aa, Mus musculus
GYSITSDY

SEQ ID NO: 87 - protein, 9 aa, Mus musculus
GYSITSDYA

SEQ ID NO: 88 - protein, 16 aa, Mus musculus
YITYSGSTNYNPSLKS

SEQ ID NO: 89 - protein, 11 aa, Mus musculus
CWDYALYAMDC

SEQ ID NO: 90 - protein, 9 aa, Mus musculus
WDYALYAMD

SEQ ID NO: 91 - protein, 13 aa, Mus musculus
ARCWDYALYAMDC

SEQ ID NO: 92 - protein, 11 aa, Mus musculus
RTSENIYSYLA

SEQ ID NO: 93 - protein, 7 aa, Mus musculus
NAKTLAE

SEQ ID NO: 94 - protein, 9 aa, Mus musculus
QHHYDTPLT

SEQ ID NO: 95 - protein, 9 aa, Mus musculus
NAKHYDTPL

SEQ ID NO: 96 - protein, 9 aa, Mus musculus
QHHYDTPLT

SEQ ID NO: 97 - protein, 5 aa, Mus musculus
NYGMN

SEQ ID NO: 98 - protein, 7 aa, Mus musculus
GYTFTNY

SEQ ID NO: 99 - protein, 8 aa, Mus musculus
GYTFTNYG

SEQ ID NO: 100 - protein, 17 aa, Mus musculus
WINTNTGEPTYAEEFKG

SEQ ID NO: 101 - protein, 8 aa, Mus musculus
INTNTGEP

SEQ ID NO: 102 - protein, 8 aa, Mus musculus
DYLYYFDY

SEQ ID NO: 103 - protein, 6 aa, Mus musculus
YLYYFD

SEQ ID NO: 104 - protein, 10 aa, Mus musculus
ARDYLYYFDY

SEQ ID NO: 105 - protein, 11 aa, Mus musculus
KASENVVTYVS

SEQ ID NO: 106 - protein, 7 aa, Mus musculus
SENVVTY

SEQ ID NO: 107 - protein, 6 aa, Mus musculus
ENVVTY

SEQ ID NO: 108 - protein, 7 aa, Mus musculus
GASNRYT

SEQ ID NO: 109 - protein, 9 aa, Mus musculus
GQGYSYPYT

SEQ ID NO: 110 - protein, 6 aa, Mus musculus
GYSYPY

SEQ ID NO: 111 - protein, 9 aa, Mus musculus
GQGYSYPYT

SEQ ID NO: 112 - protein, 227 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - DNA, 333 bp, Mus musculus
  source 1..333: mol_type = genomic DNA
gacattcagctgacccaatctccagcttctttggctgtgtctctagggcagagggccaccatctcctgcaaggccagccaaagtgttgattatgatggtgatagttatttgaactggtaccaacagataccaggacagccacccaaactcctcatctatgatgcatccaatctagtatctgggattccacccaggtttagtggcagtgggtctgggacagacttcaccctcaacatccatcctgtggagaaggtggatgctgcaacctatcactgtcagcaaagtactgaggacccttggacgttcggtggaggcaccaagctggaaatcaaa

SEQ ID NO: 114 - protein, 111 aa, Mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 115 - DNA, 372 bp, Mus musculus
  source 1..372: mol_type = other DNA
caggttcagctgcagcagtctggggctgagctggtgcggcctgggtcctcagtgaagatttcctgcaaagcatctggctacgcattcagtagctactggatgaactgggtgaagcagaggcctggacagggtcttgagtggattggacagatttggcctggagatggtgatactaactacaacggaaagttcaagggcaaggccacactgactgcagacgaatcctccagcacagcctacatgcagctcagcagcctggcctctgaggactctgcggtctatttctgtgcaagacgagaaacgaccactgtcgggcgttattactatgctatggactactggggtcaaggaaccacagtcaccgtctcctca

SEQ ID NO: 116 - protein, 124 aa, Mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 117 - DNA, 648 bp, Homo sapiens
  source 1..648: mol_type = other DNA
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccaagcccccatcccgggaggagatgaccaagaaccaggtcagcctgtcctgcctggtcaaaggcttctatcccagcgacatcgccgtggagtgggagagcaatgggcagccggagaacaactacaagaccacggttcccgtgctggactccgacggctccttccgcctcgctagctacctcaccgtggacaagagcaggtggcagcaggggaacgtcttctcatgctccgtgatgcatgaggctctgcacaaccactacacgcagaagagcctctccctgtccccgggg

SEQ ID NO: 118 - protein, 718 aa, synthetic construct
  REGION 1..718: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGSSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGSTGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 119 - protein, 249 aa, Mus musculus
STGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSVEGGSGGSGGSGGSGGVDDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 120 - protein, 251 aa, Mus musculus
STGSEVQLQESGPGLVKPSQSLSLTCTVTGYSITSDYAWNWIRQFPGNKLEWMGYITYSGSTSYNPSLESRISITRDTSTNQFFLQLNSVTTEDTATYYCARGGYYGSSWGVFAYWGQGTLVTVSAVEGGSGGSGGSGGSGGVDDIQMTQSPASLSASVGETVTITCRVSENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 121 - protein, 245 aa, Mus musculus
STGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSVEGGSGGSGGSGGSGGVDDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 122 - protein, 246 aa, Mus musculus
STGSQVQLQQSAVELARPGASVKMSCKASGYTFTSFTMHWVKQRPGQGLEWIGYINPSSGYTEYNQKFKDKTTLTADKSSSTAYMQLDSLTSDDSAVYYCVRGSSRGFDYWGQGTLVTVSAVEGGSGGSGGSGGSGGVDDIQMIQSPASLSVSVGETVTITCRASENIYSNLAWFQQKQGKSPQLLVYAATNLADGVPSRFSGSGSGTQYSLKINSLQSEDFGIYYCQHFWGTPRTFGGGTKLEIK

SEQ ID NO: 123 - protein, 245 aa, Mus musculus
STGSQVQLQQPGSVLVRPGASVKLSCKASGYTFTSSWMHWAKQRPGQGLEWIGHIHPNSGISNYNEKFKGKATLTVDTSSSTAYVDLSSLTSEDSAVYYCARGGRFDDWGAGTTVTVSSVEGGSGGSGGSGGSGGVDDIVMTQSPATLSVTPGDRVSLSCRASQSISDYLHWYQQKSHESPRLLIKYASQSISGIPSRFSGSGSGSDFTLSINSVEPEDVGVYYCQNGHSFLMYTFGGGTKLEIK

SEQ ID NO: 124 - protein, 249 aa, Mus musculus
STGSDVQLQESGPGLVKPSQSLSLTCTVTGYSITSDYAWNWIRQFPGNKLEWMGYITYSGSTNYNPSLKSRISITRDTSKNQFFLQLNSVTTEDTATYYCARCWDYALYAMDCWGQGTSVTVSSVEGGSGGSGGSGGSGGVDDIQMTQSPASLSASVGETVTITCRTSENIYSYLAWCQQKQGKSPQLLVYNAKTLAEGVPSRFSGSGSGTHFSLKINSLQPEDFGIYYCQHHYDTPLTFGAGTKLELK

SEQ ID NO: 125 - protein, 246 aa, Mus musculus
STGSQIQLVQSGPELQKPGETVKISCKASGYTFTNYGMNWVKQAPGKGLKWMGWINTNTGEPTYAEEFKGRFAFSLETSASTAYLQINNLKNEDTATYFCARDYLYYFDYWGQGTTLTVSSVEGGSGGSGGSGGSGGVDNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIK

SEQ ID NO: 126 - DNA, 751 bp, Mus musculus
  source 1..751: mol_type = other DNA
gtcgactggaagccaggtacagctgcagcagtctggccctgaactcgtcaaaccaggagcttccgtgaagatgtcctgcaaggcttcagggtacacgtttaccgactatgtgatcaattggggtaagcagcgctctgggcaaggcttggagtggattggcgagatctatcctgggagtgggaccaactattacaacgagaagttcaaggccaaagccactttgactgcagacaagagctcaaacattgcctacatgcaactgagctccctgacatcagaggattctgctgtgtacttctgtgcacgtagaggtcggtacggtctgtatgccatggactattggggccaaggcacttccgtgacagtcagctctgtggaaggaggaagtggcggttcaggaggtagcggagggtccggaggagtggatgacattcagatgacacagaccacttctagcctctccgcatcccttggggatagggtcaccatcagttgtagggctagccaggacatttccaattacctgaactggtatcagcagaaacccgatggcacagttaagcttctgatctactacacaagcagactgcactcaggggttccatctcggtttagtggaagtggctctggtaccgactattccctgaccatcaacaatctggaacaggaagatatcgccacctacttctgccaacagggcaatactcgaccctggacatttggtggcggcacgaaactcgagataaaataa

SEQ ID NO: 127 - DNA, 757 bp, Mus musculus
  source 1..757: mol_type = other DNA
gtcgactggatccgaggtacagttgcaggagagtgggcctggactggtcaaaccctcccaatctctgagcttgacatgcacagtcacaggctacagcatcacctccgactacgcttggaattggattcgacagtttcccggcaacaagctggaatggatgggctacatcacctatagtggtagcacttcctataatccctcacttgagagccggatttccatcactagggatacgagcaccaaccagttcttcctgcagctgaatagcgtcaccaccgaagatactgccacctattactgcgcaagaggcggttactatggcagttcatggggtgtattcgcctattggggacaggggacacttgtgacagtgtctgctgttgaaggtggatccggcggatcaggagggagtggtggcagtggaggtgttgacgacattcagatgacccaatcccctgcttctctctcagcctctgtgggagagactgtgaccataacctgtcgtgttagcgagaacatctactcctatctcgcctggtatcagcagaaacaggggaaatccccacaactgctcgtgtacaatgccaagactctggcagaaggagtgccaagccgcttttccgggtctgggtctgggacacagttctcactgaagatcaactctttgcaacctgaggattttggctcttactactgtcagcatcactatggcacaccatggacgtttggtggcggcactaagctggagattaagtaa

SEQ ID NO: 128 - DNA, 739 bp, Mus musculus
  source 1..739: mol_type = other DNA
gtcgactgggtccgaagtgcaactgcaacagtctggccctgagctggtcaaacccggtgcttcagtgaagatctcctgcaagacatccggctacaccttcactgagtacaccatgcactgggtcaaacagtctcacggtaagagcctggagtggataggcggaatttcacccaacattggagggacctcctataaccagaagttcaagggcaaagccacccttacagttgacaagagcagttcaactgcctacatggaactgcgctcattgacctccgaggattcagccgtgtattactgcgctagaaggggaggatccttcgattattggggccaaggcactacgcttaccgtgagcagcgttgaaggtggttctggcggctctggtggaagtggagggagtggcggggtagacgacatcgtgatgactcagagtccagcaactctgtccgttacacctggagatcgagtgtctctgagttgtcgtgcaagccagtctatctctgactatctgcactggtatcagcagaagagccatgagtcacctaggctgttgatcaagtacgcctctcagtccattagcgggattccatcccggtttagtggctctggctccggtagtgacttcacactcagcatcaatagcgtcgaaccagaggatgtaggggtgtactactgtcagaatgggcattcctttcccctcacatttggagctggtaccaaactcgagctgaaataa

SEQ ID NO: 129 - DNA, 742 bp, Mus musculus
  source 1..742: mol_type = other DNA
gtcgactggctcccaagtacagcttcagcagtctgccgtcgaacttgctcgaccaggagcttcagtgaagatgagctgcaaagcctctggttacaccttcacgtcctttaccatgcattgggtgaagcagcgtcctggccaaggcctggagtggattggctacatcaatccctccagcgggtataccgagtacaaccagaagttcaaggacaaaacaaccctgactgccgataagtcaagtagcacagcctatatgcagctggattccctgacatcagacgatagcgctgtgtattactgcgttaggggctctagcagagggttcgactattggggtcaaggcacactggtcacggttagtgccgttgaaggaggctctggaggcagtggaggttctggagggtcaggcggtgtggatgacattcagatgattcagagtcccgctagcttgagtgtaagcgtcggtgagacagtgaccatcacttgtcgcgcatccgaaaacatctactccaatctcgcatggttccagcagaaacagggcaaatcaccccaattgctcgtgtatgccgcaactaatctggctgatggtgtgccttccaggtttagcgggtctggatctgggactcagtactccctgaagatcaactccctccagtctgaggacttcgggatctattactgtcagcacttttggggaactccacggacctttggaggcgggaccaaactggagataaagtaa

SEQ ID NO: 130 - DNA, 696 bp, Mus musculus
  source 1..696: mol_type = other DNA
ctggtgaggccaggtgcatctgtgaagctgtcatgcaaagcatccgggtacacgttcacctcttcatggatgcattgggccaaacagcgtccaggccagggccttgagtggattggacacattcaccccaatagcggcatatccaactacaacgagaagttcaagggcaaagccacactgacagtggatacttccagctctacagcctatgtggaccttagtagcttgaccagtgaggattctgccgtatactactgtgctagaggtgggcggtttgacgattggggtgctgggaccacagtcaccgtgagcagtgtcgaaggtggatcaggtggatctggaggctcaggcggttctggcggtgttgacgacatcgtgatgactcaaagccctgctactctctctgtcacacccggagatagggtaagcctcagttgtcgagcaagccagtcaatcagcgactatctgcactggtatcagcagaagtcccatgaatccccacgcttgctcatcaagtacgccagtcagtccatcagtggcattccttcccggttttctgggtctggatccgggtcagacttcactctgagcattaactccgtcgaacccgaggatgttggcgtgtattattgccagaatggacattccttcctgatgtacacctttggcggagggaccaaactggagatcaagtaa

SEQ ID NO: 131 - DNA, 751 bp, Mus musculus
  source 1..751: mol_type = other DNA
gtcgactgggtctgatgtgcagttgcaggagtcaggacctgggcttgtcaagccaagccagagcctcagtctcacttgcactgtcacaggctatagcatcacatccgactatgcttggaattggattaggcagtttcctggcaataagctggaatggatggggtacatcacctattccggcagtaccaactacaatcccagcttgaaatctcggatttccataacacgcgatactagcaagaaccagttcttccttcagctgaactctgtgacaacagaggataccgctacgtactattgcgccagatgctgggattacgccctgtatgccatggactgttggggtcaaggtaccagcgttactgtgtctagcgtcgaaggcggaagtggcggctcaggagggtcaggaggctcaggcggagtggatgacattcagatgacccaatctcccgcatccctgtccgcatcagtaggggagacagtgaccattacctgtcgtacttccgagaacatctactcctatcttgcctggtgtcaacagaaacaggggaaaagtccacagctgctggtgtataacgccaagaccttggcagaaggtgttcccagtcgattctctggttccggatccggtacacacttcagcctgaagatcaattctctgcaaccagaggactttggaatctactactgccagcatcactacgacactcctctgacgtttggcgctggtaccaagctcgaactgaaataa

SEQ ID NO: 132 - DNA, 742 bp, Mus musculus
  source 1..742: mol_type = other DNA
gtcgactggtagccagatacagctggtacagtcaggaccagagctgcagaaacctggagagacagtgaagatcagctgcaaggctagcgggtacaccttcacgaattacgggatgaactgggtcaagcaggctccaggcaaagggctgaagtggatgggctggattaacaccaatactggggaaccaacctatgccgaggaattcaaggggagatttgccttttccctcgaaaccagcgcctcaaccgcctatctccagatcaacaacctgaagaatgaggataccgctacctacttctgtgcaagggactacctctactacttcgactattggggccaaggtacgactcttacagtctctagtgttgagggagggagtggaggttctggaggctctggtggctctggaggcgttgacaacatcgtgatgactcagtctcccaaaagcatgagtatgagtgtgggtgaacgagttaccttgacatgcaaagcctccgagaatgtcgtgacatacgtgtcctggtatcagcagaaacccgagcaatcccctaagctgctgatctatggcgctagcaatcgctatactggcgtacctgatcggttcacaggatcaggctcagccactgactttactcttaccatttcctccgtgcaggcagaagatttggcagattaccactgtgggcaaggttactcttatccctatacatttggaggcggcacaaagctggagattaagtaa

SEQ ID NO: 133 - protein, 720 aa, synthetic construct
  REGION 1..720: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGSSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGSTGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSVEGGSGGSGGSGGSGGVDDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 134 - protein, 722 aa, synthetic construct
  REGION 1..722: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGSSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGSTGSEVQLQESGPGLVKPSQSLSLTCTVTGYSITSDYAWNWIRQFPGNKLEWMGYITYSGSTSYNPSLESRISITRDTSTNQFFLQLNSVTTEDTATYYCARGGYYGSSWGVFAYWGQGTLVTVSAVEGGSGGSGGSGGSGGVDDIQMTQSPASLSASVGETVTITCRVSENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 135 - protein, 716 aa, synthetic construct
  REGION 1..716: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGSSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSVEGGSGGSGGSGGSGGVDDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 136 - protein, 717 aa, synthetic construct
  REGION 1..717: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGSSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGSTGSQVQLQQSAVELARPGASVKMSCKASGYTFTSFTMHWVKQRPGQGLEWIGYINPSSGYTEYNQKFKDKTTLTADKSSSTAYMQLDSLTSDDSAVYYCVRGSSRGFDYWGQGTLVTVSAVEGGSGGSGGSGGSGGVDDIQMIQSPASLSVSVGETVTITCRASENIYSNLAWFQQKQGKSPQLLVYAATNLADGVPSRFSGSGSGTQYSLKINSLQSEDFGIYYCQHFWGTPRTFGGGTKLEIK

SEQ ID NO: 137 - protein, 716 aa, synthetic construct
  REGION 1..716: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGSSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGSTGSQVQLQQPGSVLVRPGASVKLSCKASGYTFTSSWMHWAKQRPGQGLEWIGHIHPNSGISNYNEKFKGKATLTVDTSSSTAYVDLSSLTSEDSAVYYCARGGRFDDWGAGTTVTVSSVEGGSGGSGGSGGSGGVDDIVMTQSPATLSVTPGDRVSLSCRASQSISDYLHWYQQKSHESPRLLIKYASQSISGIPSRFSGSGSGSDFTLSINSVEPEDVGVYYCQNGHSFLMYTFGGGTKLEIK

SEQ ID NO: 138 - protein, 720 aa, synthetic construct
  REGION 1..720: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGSSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGSTGSDVQLQESGPGLVKPSQSLSLTCTVTGYSITSDYAWNWIRQFPGNKLEWMGYITYSGSTNYNPSLKSRISITRDTSKNQFFLQLNSVTTEDTATYYCARCWDYALYAMDCWGQGTSVTVSSVEGGSGGSGGSGGSGGVDDIQMTQSPASLSASVGETVTITCRTSENIYSYLAWCQQKQGKSPQLLVYNAKTLAEGVPSRFSGSGSGTHFSLKINSLQPEDFGIYYCQHHYDTPLTFGAGTKLELK

SEQ ID NO: 139 - protein, 717 aa, synthetic construct
  REGION 1..717: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGSSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGSTGSQIQLVQSGPELQKPGETVKISCKASGYTFTNYGMNWVKQAPGKGLKWMGWINTNTGEPTYAEEFKGRFAFSLETSASTAYLQINNLKNEDTATYFCARDYLYYFDYWGQGTTLTVSSVEGGSGGSGGSGGSGGVDNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIK

SEQ ID NO: 140 - protein, 698 aa, synthetic construct
  REGION 1..698: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGSSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTH

SEQ ID NO: 141 - protein, 214 aa, synthetic construct
  REGION 1..214: note = Chimeric homo sapiens mus musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 837 aa, synthetic construct
  REGION 1..837: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGSSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSVEGGSGGSGGSGGSGGVDDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 143 - protein, 837 aa, synthetic construct
  REGION 1..837: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGSSAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSVEGGSGGSGGSGGSGGVDDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 144 - protein, 218 aa, synthetic construct
  REGION 1..218: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 675 aa, synthetic construct
  REGION 1..675: note = Chimeric homo sapiens mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGSSAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 214 aa, synthetic construct
  REGION 1..214: note = Chimeric homo sapiens mus musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTH

SEQ ID NO: 147 - protein, 218 aa, synthetic construct; identical to SEQ ID NO: 144
  REGION 1..218: note = Chimeric homo sapiens mus musculus

SEQ ID NO: 148 - protein, 801 aa, synthetic construct
  REGION 1..801: note = Chimeric homo sapiens mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 214 aa, synthetic construct; identical to SEQ ID NO: 146
  REGION 1..214: note = Chimeric homo sapiens mus musculus

SEQ ID NO: 150 - protein, 218 aa, synthetic construct; identical to SEQ ID NO: 144
  REGION 1..218: note = Chimeric homo sapiens mus musculus

SEQ ID NO: 151 - protein, 801 aa, synthetic construct; identical to SEQ ID NO: 148
  REGION 1..801: note = Chimeric homo sapiens mus musculus

SEQ ID NO: 152 - protein, 214 aa, synthetic construct; identical to SEQ ID NO: 146
  REGION 1..214: note = Chimeric homo sapiens mus musculus

SEQ ID NO: 153 - protein, 218 aa, synthetic construct; identical to SEQ ID NO: 144
  REGION 1..218: note = Chimeric homo sapiens mus musculus

SEQ ID NO: 154 - protein, 796 aa, synthetic construct
  REGION 1..796: note = Chimeric homo sapiens mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGSSAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 214 aa, synthetic construct; identical to SEQ ID NO: 146
  REGION 1..214: note = Chimeric homo sapiens mus musculus

SEQ ID NO: 156 - protein, 218 aa, synthetic construct; identical to SEQ ID NO: 144
  REGION 1..218: note = Chimeric homo sapiens mus musculus

SEQ ID NO: 157 - protein, 819 aa, synthetic construct
  REGION 1..819: note = Chimeric homo sapiens mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSVEGGSGGSGGSGGSGGVDDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 158 - protein, 218 aa, synthetic construct; identical to SEQ ID NO: 144
  REGION 1..218: note = Chimeric homo sapiens mus musculus

SEQ ID NO: 159 - protein, 819 aa, synthetic construct
  REGION 1..819: note = Chimeric homo sapiens mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPSPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSVEGGSGGSGGSGGSGGVDDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 160 - protein, 218 aa, synthetic construct; identical to SEQ ID NO: 144
  REGION 1..218: note = Chimeric homo sapiens mus musculus

SEQ ID NO: 161 - protein, 815 aa, synthetic construct
  REGION 1..815: note = Chimeric homo sapiens mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGSSPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSVEGGSGGSGGSGGSGGVDDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 162 - protein, 819 aa, synthetic construct
  REGION 1..819: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGSSAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - protein, 214 aa, synthetic construct; identical to SEQ ID NO: 146
  REGION 1..214: note = Chimeric homo sapiens mus musculus

SEQ ID NO: 164 - protein, 819 aa, synthetic construct
  REGION 1..819: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGSSAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTH

SEQ ID NO: 165 - protein, 214 aa, synthetic construct; identical to SEQ ID NO: 141
  REGION 1..214: note = Chimeric homo sapiens mus musculus

SEQ ID NO: 166 - protein, 218 aa, synthetic construct; identical to SEQ ID NO: 144
  REGION 1..218: note = Chimeric homo sapiens mus musculus

SEQ ID NO: 167 - protein, 792 aa, synthetic construct
  REGION 1..792: note = Chimeric homo sapiens mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPSPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTH

SEQ ID NO: 168 - protein, 223 aa, synthetic construct
  REGION 1..223: note = Chimeric homo sapiens mus musculus
EVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 445 aa, synthetic construct
  REGION 1..445: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 680 aa, synthetic construct
  REGION 1..680: note = Chimeric homo sapiens mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 214 aa, synthetic construct; identical to SEQ ID NO: 146
  REGION 1..214: note = Chimeric homo sapiens mus musculus

SEQ ID NO: 172 - protein, 445 aa, synthetic construct
  REGION 1..445: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 680 aa, synthetic construct
  REGION 1..680: note = Chimeric homo sapiens mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 214 aa, synthetic construct; identical to SEQ ID NO: 146
  REGION 1..214: note = Chimeric homo sapiens mus musculus

SEQ ID NO: 175 - protein, 445 aa, synthetic construct
  REGION 1..445: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 680 aa, synthetic construct
  REGION 1..680: note = Chimeric homo sapiens mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - protein, 214 aa, synthetic construct; identical to SEQ ID NO: 146
  REGION 1..214: note = Chimeric homo sapiens mus musculus

SEQ ID NO: 178 - protein, 445 aa, synthetic construct; identical to SEQ ID NO: 169
  REGION 1..445: note = Chimeric homo sapiens mus musculus

SEQ ID NO: 179 - protein, 698 aa, synthetic construct
  REGION 1..698: note = Chimeric homo sapiens mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSVEGGSGGSGGSGGSGGVDDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 180 - protein, 445 aa, synthetic construct; identical to SEQ ID NO: 172
  REGION 1..445: note = Chimeric homo sapiens mus musculus

SEQ ID NO: 181 - protein, 698 aa, synthetic construct
  REGION 1..698: note = Chimeric homo sapiens mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSVEGGSGGSGGSGGSGGVDDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 182 - protein, 454 aa, synthetic construct
  REGION 1..454: note = Chimeric homo sapiens mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 670 aa, synthetic construct
  REGION 1..670: note = Chimeric homo sapiens mus musculus
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 214 aa, synthetic construct; identical to SEQ ID NO: 146
  REGION 1..214: note = Chimeric homo sapiens mus musculus

SEQ ID NO: 185 - protein, 445 aa, synthetic construct; identical to SEQ ID NO: 172
  REGION 1..445: note = Chimeric homo sapiens mus musculus

SEQ ID NO: 186 - protein, 671 aa, synthetic construct
  REGION 1..671: note = Chimeric homo sapiens mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTH

SEQ ID NO: 187 - protein, 223 aa, synthetic construct; identical to SEQ ID NO: 168
  REGION 1..223: note = Chimeric homo sapiens mus musculus

SEQ ID NO: 188 - protein, 680 aa, synthetic construct
  REGION 1..680: note = Human-mouse chimeric
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 189 - protein, 446 aa, synthetic construct
  REGION 1..446: note = Human-mouse chimeric
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 459 aa, synthetic construct
  REGION 1..459: note = Human-mouse chimeric
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHGGSSSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSVEGGSGGSGGSGGSGGVDDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 191 - protein, 446 aa, synthetic construct
  REGION 1..446: note = Human-mouse chimeric
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 680 aa, synthetic construct
  REGION 1..680: note = Human mouse chimeric
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 193 - protein, 459 aa, synthetic construct; identical to SEQ ID NO: 190
  REGION 1..459: note = Human mouse chimeric

SEQ ID NO: 194 - protein, 801 aa, synthetic construct
  REGION 1..801: note = Human-mouse chimeric
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPSPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 219 aa, synthetic construct
  REGION 1..219: note = Human-mouse chimeric
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 459 aa, synthetic construct; identical to SEQ ID NO: 190
  REGION 1..459: note = Human - mouse chimeric

SEQ ID NO: 197 - protein, 928 aa, synthetic construct
  REGION 1..928: note = Human-mouse chimeric
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKGGGGSGGGGSGGGGSQVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPSPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 459 aa, synthetic construct; identical to SEQ ID NO: 190
  REGION 1..459: note = Human - mouse chimeric

SEQ ID NO: 199 - protein, 120 aa, synthetic construct
  REGION 1..120: note = Chimeric
QVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSS

SEQ ID NO: 200 - protein, 120 aa, synthetic construct
  REGION 1..120: note = Chimeric
QVQLVQSGAEVKKPGSSVKVSCKASGYTFTDYVINWGRQAPGQGLEWIGEIYPGSGTNYYNEKFKAKATITADKSTSTAYMELSSLRSEDTAVYFCARRGRYGLYAMDYWGQGTTVTVSS

SEQ ID NO: 201 - protein, 107 aa, synthetic construct
  REGION 1..107: note = Chimeric
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIK

SEQ ID NO: 202 - protein, 122 aa, synthetic construct
  REGION 1..122: note = Chimeric
QVQLQESGPGLVKPSQTLSLTCTVSGYSISSDYAWNWIRQPPGKGLEWIGYITYSGSTSYNPSLESRVTISRDTSKNQFSLKLSSVTAADTAVYYCARGGYYGSSWGVFAYWGQGTLVTVSS

SEQ ID NO: 203 - protein, 122 aa, synthetic construct
  REGION 1..122: note = Chimeric
QVQLQESGPGLVKPSQTLSLTCTVSGYSISSDYAWNWIRQPPGKGLEWMGYITYSGSTSYNPSLESRITISRDTSKNQFSLKLSSVTAADTAVYYCARGGYYGSSWGVFAYWGQGTLVTVSS

SEQ ID NO: 204 - protein, 122 aa, synthetic construct
  REGION 1..122: note = Chimeric
QVQLQESGPGLVKPSQTLSLTCTVSGYSITSDYAWNWIRQPPGKGLEWMGYITYSGSTSYNPSLESRITISRDTSKNQFSLKLSSVTAADTAVYYCARGGYYGSSWGVFAYWGQGTLVTVSS

SEQ ID NO: 205 - protein, 107 aa, synthetic construct
  REGION 1..107: note = Chimeric
DIQMTQSPSSLSASVGDRVTITCRVSENIYSYLAWYQQKPGKAPKLLVYNAKTLAEGVPSRFSGSGSGTDFTLTISSLQPEDFATYYCQHHYGTPWTFGGGTKVEIK

SEQ ID NO: 206 - protein, 116 aa, synthetic construct
  REGION 1..116: note = Chimeric
QVQLVQSGAEVKKPGSSVKVSCKASGYTFSEYTMHWVRQAPGQGLEWMGGISPNIGGTSYNQKFKGRVTITADKSTSTAYMELSSLRSEDTAVYYCARRGGSFDYWGQGTTVTVSS

SEQ ID NO: 207 - protein, 116 aa, synthetic construct
  REGION 1..116: note = Chimeric
QVQLVQSGAEVKKPGSSVKVSCKASGYTFSEYTMHWVRQAPGQGLEWIGGISPNIGGTSYNQKFKGRATITADKSTSTAYMELSSLRSEDTAVYYCARRGGSFDYWGQGTTVTVSS

SEQ ID NO: 208 - protein, 116 aa, synthetic construct
  REGION 1..116: note = Chimeric
QVQLVQSGAEVKKPGSSVKVSCKASGYTFSEYTMHWVRQAPGQGLEWIGGISPNIGGTSYNQKFKGRATLTADKSTSTAYMELSSLRSEDTAVYYCARRGGSFDYWGQGTTVTVSS

SEQ ID NO: 209 - protein, 107 aa, synthetic construct
  REGION 1..107: note = Chimeric
EIVMTQSPATLSVSPGERATLSCRASQSISDYLHWYQQKPGQAPRLLIKYASQSISGIPARFSGSGSGTDFTLTISSLEPEDFAVYYCQNGHSFPLTFGQGTKLEIK

SEQ ID NO: 210 - protein, 117 aa, synthetic construct
  REGION 1..117: note = Chimeric
QVQLVQSGAEVKKPGASVKVSCKASGYTFTSFTMHWVRQAPGQGLEWIGYINPSSGYTEYNQKFKDRVTITADKSTSTAYMELSSLRSEDTAVYYCVRGSSRGFDYWGQGTLVTVSS

SEQ ID NO: 211 - protein, 117 aa, synthetic construct
  REGION 1..117: note = Chimeric
QVQLVQSGAEVKKPGASVKVSCKASGYTFTSFTMHWVRQAPGQGLEWIGYINPSSGYTEYNQKFKDRTTITADKSTSTAYMELSSLRSEDTAVYYCVRGSSRGFDYWGQGTLVTVSS

SEQ ID NO: 212 - protein, 117 aa, synthetic construct
  REGION 1..117: note = Chimeric
QVQLVQSGAEVKKPGASVKVSCKASGYTFTSFTMHWVRQAPGQGLEWIGYINPSSGYTEYNQKFKDRTTLTADKSTSTAYMELSSLRSEDTAVYYCVRGSSRGFDYWGQGTLVTVSS

SEQ ID NO: 213 - protein, 107 aa, synthetic construct
  REGION 1..107: note = Chimeric
DIQMTQSPSSLSASVGDRVTITCRASENIYSNLAWFQQKPGKAPKLLVYAATNLADGVPSRFSGSGSGTDYTLTISSLQPEDFATYYCQHFWGTPRTFGGGTKVEIK

SEQ ID NO: 214 - protein, 120 aa, synthetic construct
  REGION 1..120: note = Chimeric
QVQLQESGPGLVKPSQTLSLTCTVSGGSISSDYAWNWIRQPPGKGLEWIGYITYSGSTNYNPSLKSRVTISRDTSKNQFSLKLSSVTAADTAVYYCARCWDYALYAMDCWGQGTTVTVSS

SEQ ID NO: 215 - protein, 120 aa, synthetic construct
  REGION 1..120: note = Chimeric
QVQLQESGPGLVKPSQTLSLTCTVSGYSISSDYAWNWIRQPPGKGLEWIGYITYSGSTNYNPSLKSRVTISRDTSKNQFSLKLSSVTAADTAVYYCARCWDYALYAMDCWGQGTTVTVSS

SEQ ID NO: 216 - protein, 120 aa, synthetic construct
  REGION 1..120: note = Chimeric
QVQLQESGPGLVKPSQTLSLTCTVSGYSISSDYAWNWIRQPPGKGLEWMGYITYSGSTNYNPSLKSRITISRDTSKNQFSLKLSSVTAADTAVYYCARCWDYALYAMDCWGQGTTVTVSS

SEQ ID NO: 217 - protein, 107 aa, synthetic construct
  REGION 1..107: note = Chimeric
DIQMTQSPSSLSASVGDRVTITCRTSENIYSYLAWCQQKPGKAPKLLIYNAKTLAEGVPSRFSGSGSGTDFTLTISSLQPEDFATYYCQHHYDTPLTFGQGTKLEIK

SEQ ID NO: 218 - protein, 107 aa, synthetic construct
  REGION 1..107: note = Chimeric
DIQMTQSPSSLSASVGDRVTITCRTSENIYSYLAWCQQKPGKAPKLLVYNAKTLAEGVPSRFSGSGSGTDFTLTISSLQPEDFATYYCQHHYDTPLTFGQGTKLEIK

(19) United States
(12) Patent Application Publication
(10) Pub. No.: US 2024/0174748 A1
(43) Pub. Date: May 30, 2024

(54) VARIABLE REGIONS FOR NKP46 BINDING PROTEINS

(71) Applicant: INNATE PHARMA, MARSEILLE (FR)

(72) Inventors: LAURENT GAUTHIER, MARSEILLE (FR); NADIA ANCERIZ, AUBAGNE (FR); ARIANE MOREL, MARSEILLE (FR); BENJAMIN ROSSI, MARSEILLE (FR)

(21) Appl. No.: 18/432,126

(22) Filed: Feb. 5, 2024

Related U.S. Application Data
(62) Division of application No. 17/315,547, filed on May 10, 2021, now Pat. No. 11,891,444.
Application No. 17/315,547 is a division of application No. 16/066,688, filed on Jun. 28, 2018, now Pat. No. 11,001,629.
(60) Provisional application No. 62/271,474, filed on Dec. 28, 2015.

(51) Int. Cl. C07K 16/28; C07K 16/46; C12N 5/10; C12N 15/00; C12N 15/64; C12N 15/66

(52) U.S. Cl. CPC C07K 16/2803; C07K 16/2809; C07K 16/2887; C07K 16/468; C12N 5/10; C12N 15/00; C12N 15/64; C12N 15/66; C07K 2317/24; C07K 2317/31; C07K 2317/33; C07K 2317/34; C07K 2317/526; C07K 2317/622; C07K 2317/732; C07K 2317/92; C12N 2511/00; C12N 2523/00

(57) ABSTRACT

The application pertains to NKp46-binding immunoglobulin variable regions, and proteins such as antibodies and multispecific proteins that comprise the variable regions. The proteins can bind and specifically redirect NK cells to lyse a target cell of interest. The proteins have utility in the treatment of disease, notably cancer or infectious disease.

### CROSS-REFERENCE TO RELATED APPLICATIONS

This application is a divisional of U.S. application Ser. No. 17/315,547, filed May 10, 2021, now U.S. Pat. No. 11,891,444, which is a divisional of U.S. application Ser. No. 16/066,688, filed Jun. 28, 2018, now U.S. Pat. No. 11,001,629, which is the U.S. national stage application of International Patent Application No. PCT/EP2016/081953, filed Dec. 20, 2016, which claims the benefit of U.S. Provisional Application No. 62/271,474, filed Dec. 28, 2015, which are incorporated herein by reference in their entirety; including any drawings and sequence listings.

### REFERENCE TO THE SEQUENCE LISTING

The present application is being filed along with a Sequence Listing in electronic format. The Sequence Listing is provided as a file entitled “Seq-List.xml”, created Jan. 19, 2024, which is 269,012 bytes in size. The information in the electronic format of the Sequence Listing is incorporated herein by reference in its entirety.

### FIELD OF THE INVENTION

Immunoglobulin variable regions, and proteins such as antibodies and multispecific proteins that comprise the variable regions are provided. The proteins can bind and specifically redirect NK cells to lyse a target cell of interest. The proteins have utility in the treatment of disease, notably cancer or infectious disease.

### BACKGROUND

Bispecific antibodies binding two different epitopes offer opportunities for increasing specificity, broadening potency, and utilizing novel mechanisms of action that cannot be achieved with a traditional monoclonal antibody. A variety of formats for bispecific antibodies that bind to two targets simultaneously have been reported. Cross-linking two different receptors using a bispecific antibody to inhibit a signaling pathway has shown utility in a number of applications (see, e.g., Jackman, et al., (2010) J. Biol. Chem. 285:20850-20859). Bispecific antibodies have also been used to neutralize two different receptors. In other approaches, bispecific antibodies have been used to recruit immune effector cells, where T-cell activation is achieved in proximity to tumor cells by the bispecific antibody which binds receptors simultaneously on the two different cell types (see Baeuerle, P. A., et al, (2009) Cancer Res 69(12):4941-4). Approaches developed to date have primarily involved bispecific antibodies that link the CD3 complex on T cells to a tumor-associated antigen. However in other examples, bispecific antibodies having one arm which binds CD16 (FcyinRIIIa) and another which bound to an antigen of interest such as CD19 have been developed (see Kellner et al. (2011) Cancer Lett. 303(2): 128-139).

Natural killer (NK) cells are a subpopulation of lymphocytes that are involved in non-conventional immunity. NK cells provide an efficient immunosurveillance mechanism by which undesired cells such as tumor or virally-infected cells can be eliminated. Characteristics and biological properties of NK cells include the expression of surface antigens including CD16, CD56 and/or CD57, the absence of the alpha/beta or gamma/delta TCR complex on the cell surface; the ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

NK cell activity is regulated by a complex mechanism that involves both activating and inhibitory signals. Several distinct NK cell receptors have been identified that play an important role in the NK cell mediated recognition and killing of HLA Class I deficient target cells. One receptor, although not specific to NK cells, is FcγR3a (CD16) which is responsible for NK cell mediated ADCC. Another NK cell receptor is NKp46, a member of the Ig superfamily. It is specific to NK cells and its cross-linking, induced by specific mAbs, leads to a strong NK cell activation resulting in increased intracellular Ca⁺⁺ levels, triggering of cytotoxicity, and lymphokine release. International patent publication number WO2005/105858 (Innate Pharma) discloses use of monospecific full-length IgG anti-NKp46 antibodies that bind Fcy receptors for treating hematological malignancies that are Fcγ-positive. Fc gamma receptors on tumor cells (e.g. B cell malignancies) were proposed to interact with the Fc domain of the anti-NKp46 antibodies which bound NK cells, such that the activated NK cells are brought into close proximity with their target cells via the two reactive portions of the antibody (e.g. the antigen-recognizing domain and the Fc domain), thereby enhancing the efficiency of the treatment.

To date, no NK cell specific bispecific antibodies have been developed. The depleting agents that recruit NK cytotoxicity such as anti-tumor antibodies are typically full-length IgG1 that mediate ADCC via CD16. Despite the existence of a variety of formats for bispecific antibodies, there remains a need in the art for proteins with new and well-defined mechanisms of action that can provide benefits over and can be used in addition to full-length antibodies.

### SUMMARY OF THE INVENTION

In one aspect, the present invention arises from the discovery of antibody hypervariable regions that cross-react with both a human NKp46 polypeptide and a non-human primate (e.g. e.g. a Macaca fascicularis) NKp46 polypeptide. In another aspect, the present invention arises from the discovery of antibody hypervariable regions that remain functional in in single chain proteins and multi-specific proteins (e.g. a polypeptide, a single chain protein, a multi-chain protein, including but not limited to antibody-based protein formats) that bind to NKp46 on NK cells.

Provided also are the epitopes bound by the antigen binding domains. The antigen domains bind to epitopes on NKp46 that provide for highly potent antigen binding proteins. The epitopes are furthermore shared by human and non-human primates, including on NKp46 as expressed at the surface of an NK cell.

The variable regions, when incorporated into a polypeptide (e.g. antibody, Fc protein, scFv, etc.) that binds NKp46 in monovalent manner, permit the binding to NKp46 on isolated NK cells without triggering NKp46 activation (in the absence of target cells). The variable regions are capable of binding NKp46 with high affinity as single chain polypeptides, e.g., as tandem variable regions separated by a peptide linker, as well as in F(ab) form, and are capable, as monovalent binders, to bind to NKp46 epitopes that enable NK cells to be directed to lyse target cells. Such properties make them advantageous in a variety of applications, including in particular, in multispecific (e.g. bispecific) proteins that bind to NKp46 and an antigen of interest via the heavy and light chain anti-NKp46 variable regions, and to an antigen of interest on a target cell via a hypervariable region (e.g. a heavy and light chain variable region) that specifically binds the antigen of interest. Such a multispecific protein is capable of redirecting NK cells to lyse a target cell that expresses the antigen of interest, e.g. a cell that contributes to disease. It will be appreciated that while the hypervariable regions and humanized variable regions disclosed herein retain monovalent binding in single chain form and can be used advantageously in configurations where they are placed on a single polypeptide chain, they can also be used in other applications, such as other proteins where variable regions are on separate chains, e.g. bispecific antibodies and Fc proteins, or more generally monospecific and/or conventional anti-NKp46 antibodies, for example to bind, modulate and/or detect human and/or non-human primate NKp46 polypeptides, in vivo or in vitro (e.g. in a biological sample).

In one aspect, provided is an antigen binding domain (ABD) that binds both a human and a non-human primate NKp46 polypeptide (e.g. with similar affinity, as assessed for example by surface plasmon resonance and/or by flow cytometry to NKp46-expression cells, by the methods herein). In one embodiment, the ABD is capable of binding to a NKp46 polypeptide as a single chain antigen binding domain (e.g. an scFv). In one embodiment, the ABD comprises (or is comprised in) an immunoglobulin heavy and light variable region, e.g. wherein the heavy and light chain variable domains are placed on a single polypeptide chain. In another embodiment heavy and light chain variable domains are placed on different polypeptide chains within a multimeric protein. The heavy chain variable region comprises a heavy chain framework region of human origin and the light chain variable region comprises a light chain framework regions of human origin, optionally wherein the heavy and/or light chain framework regions comprise one or more amino acid modifications (e.g. substitution(s), back-mutation(s) in which a residue is substituted by the residue present in the parental (e.g., murine) framework at the position), optionally wherein the amino acid modifications provides or increases binding to a non-human primate NKp46. In one embodiment, provided is a protein, an Fc protein, an antibody or an antibody fragment that comprises such an ABD and/or variable regions. In one aspect, provided is an isolated and/or recombinant nucleic acid that encodes such an antigen binding domain (ABD).

Examples of such proteins or polypeptides include, e.g. a single polypeptide chain NKp46-binding domain (an ABD that binds human NKp46 placed on a single polypeptide chain, e.g., a polypeptide comprising a VH and VL of the separated by a (poly)peptide linker). In one embodiment, the single chain NKp46 binding domain comprises a VH and a VL domain disclosed herein, separated by a peptide linker. The single polypeptide chain may be comprised in a multi-chain polypeptide that comprises one or more further polypeptide chains, or may be isolated as a single polypeptide chain. Another example of a protein or polypeptide that comprises an anti-NKp46 ABD is a multimeric protein comprising a first and a second polypeptide chain, wherein one chain comprises a VH domain of an anti-NKp46 ABD disclosed herein and the other chain comprises the VL of an anti-NKp46 ABD disclosed herein, wherein the chains are configured so that the VH and VL form an antigen binding domain that binds NKp46.

In one aspect, provided is a protein or polypeptide (e.g. a monospecific, bispecific, or multispecific antibody or protein, a scFv, a F(ab) or F(ab)₂, a multispecific Fc protein) that binds a human NKp46 polypeptide, optionally that further bind a non-human primate NKp46 polypeptide, wherein the protein nor polypeptide comprises: (a) a heavy chain variable region comprising a human heavy chain framework region (FR1, FR2, and FR3) derived from a human IGHV1-69 gene, and heavy chain CDR1, 2 and 3 of a NKp46-1 antibody; and (b) a light chain variable region comprising a human light chain framework region (FR1, FR2, and FR3) derived from a human IGKV1-33 gene, and light chain CDR1, 2 and 3 of a NKp46-1 antibody. Optionally, CDRs as defined by Kabat, Chotia or IMGT numbering. In one embodiment, the protein or polypeptide binds a wild-type NKp46 polypeptide but has decreased binding to a mutant NKp46 polypeptide having a mutation (e.g., an alanine substitution) any one or more of the residues K41, E42, E119, Y121 and/or Y194 (with reference to SEQ ID NO:1) compared to binding to the wild-type NKp46). In one embodiment, the heavy chain variable region comprises an amino acid sequence sharing at least 70%, 80%, 90%, 95% or 98% identity with the amino acid sequence of SEQ ID NO: 3, and/or the light chain variable region comprises an amino acid sequence sharing at least 70%, 80%, 90%, 95% or 98% identity with the amino acid sequence of SEQ ID NO: 4. In one embodiment, the heavy chain variable region comprises an amino acid sequence of the NKp46-1 H1 or H3 variable domain (or an amino acid sequence sharing at least 80, 90%, 95% or 98% identity therewith) and the light chain variable region comprises an amino acid sequence of the NKp46-1 L1 variable domain (or an amino acid sequence sharing at least 80, 90%, 95% or 98% identity therewith).

In one aspect, provided is a protein or polypeptide (e.g. a monospecific, bispecific, or multispecific antibody or protein, a scFv, a F(ab) or F(ab)₂, a multispecific Fc protein) that binds a human NKp46 polypeptide, optionally that further bind a non-human primate NKp46 polypeptide, wherein the protein nor polypeptide comprises: (a) a heavy chain variable region comprising a human heavy chain framework region (FR1, FR2, and FR3) derived from a IGHV4-30-4 gene, and heavy chain CDR1, 2 and 3 of a NKp46-2 antibody; and (b) a light chain variable region comprising a human light chain framework region (FR1, FR2, and FR3) derived from a IGKV1-39 gene, and light chain CDR1, 2 and 3 of a NKp46-2 antibody.

Optionally, CDRs as defined by Kabat, Chotia or IMGT numbering. In one embodiment, the heavy chain variable region comprises an amino acid sequence sharing at least 70%, 80%, 90%, 95% or 98% identity with the amino acid sequence of SEQ ID NO: 5, and/or the light chain variable region comprises an amino acid sequence sharing at 70%, 80%, 90%, 95% or 98% identity with the amino acid sequence of SEQ ID NO: 6. In one embodiment, the heavy chain variable region comprises an amino acid sequence of the NKp46-2 H1, H2 or H3 variable domain (or an amino acid sequence sharing at least 80, 90%, 95% or 98% identity therewith) and the light chain variable region comprises an amino acid sequence of the NKp46-2 L1 variable domain(or an amino acid sequence sharing at least 80, 90%, 95% or 98% identity therewith).

In one aspect, provided is a protein or polypeptide (e.g. a monospecific, bispecific, or multispecific antibody or protein, a scFv, a F(ab) or F(ab)₂, a multispecific Fc protein) that binds a human NKp46 polypeptide, optionally that further bind a non-human primate NKp46 polypeptide, wherein the protein nor polypeptide comprises: (a) a heavy chain variable region comprising a human heavy chain framework region (FR1, FR2, and FR3) derived from a IGHV1-69 gene, and heavy chain CDR1, 2 and 3 of a NKp46-3 antibody; and (b) a light chain variable region comprising a human light chain framework region (FR1, FR2, and FR3) derived from a IGKV3-11 and/or a IGKV3-15 gene (e.g. a mosaic variable region comprising both IGKV3-11 and IGKV3-15 sequences or segments), and light chain CDR1, 2 and 3 of a NKp46-3 antibody. Optionally, CDRs as defined by Kabat, Chotia or IMGT numbering. In one embodiment, the protein or polypeptide binds a wild-type NKp46 polypeptide but has decreased binding to a mutant NKp46 polypeptide having a mutation (e.g., an alanine substitution) any one or more of the residues P132, E133, 1135, and/or S136 (with reference to SEQ ID NO:1) compared to binding to the wild-type NKp46). In one embodiment, the heavy chain variable region comprises an amino acid sequence sharing at least 70%, 80%, 90%, 95% or 98% identity with the amino acid sequence of SEQ ID NO: 7, and/or the light chain variable region comprises an amino acid sequence sharing at least 70%, 80%, 90%, 95% or 98% identity with the amino acid sequence of SEQ ID NO: 8. In one embodiment, the heavy chain variable region comprises an amino acid sequence of the NKp46-3 H1, H3 or H4 variable domain (or an amino acid sequence sharing at least 80, 90%, 95% or 98% identity therewith) and the light chain variable region comprises an amino acid sequence of the NKp46-3 L1 variable domain (or an amino acid sequence sharing at least 80, 90%, 95% or 98% identity therewith).

In one aspect, provided is a protein or polypeptide (e.g. a monospecific, bispecific, or multispecific antibody or protein, a scFv, a F(ab) or F(ab)₂, a multispecific Fc protein) that binds a human NKp46 polypeptide, optionally that further bind a non-human primate NKp46 polypeptide, wherein the protein nor polypeptide comprises: (a) a heavy chain variable region comprising a human heavy chain framework region (FR1, FR2, and FR3) derived from a IGHV1-46 and/or a IGHV1-69 gene (e.g. a mosaic variable region comprising both IGHV1-46 and IGHV1-69 sequences or segments), and heavy chain CDR1, 2 and 3 of a NKp46-4 antibody; and (b) a light chain variable region comprising a human light chain framework region (FR1, FR2, and FR3) derived from a IGKV1-NL1 gene, and light chain CDR1, 2 and 3 of a NKp46-4 antibody. Optionally, CDRs as defined by Kabat, Chotia or IMGT numbering. In one embodiment, the protein or polypeptide binds a wild-type NKp46 polypeptide but has decreased binding to a mutant NKp46 polypeptide having a mutation (e.g., an alanine substitution) any one or more of the residues R101, V102, E104 and/or L105 (with reference to SEQ ID NO:1) compared to binding to the wild-type NKp46). In one embodiment, the heavy chain variable region comprises an amino acid sequence sharing at least 70%, 80%, 90%, 95% or 98% identity with the amino acid sequence of SEQ ID NO: 9, and/or the light chain variable region comprises an amino acid sequence sharing at least 70%, 80%, 90%, 95% or 98% identity with the amino acid sequence of SEQ ID NO: 10. In one embodiment, the heavy chain variable region comprises an amino acid sequence of the NKp46-4 H1 variable domain (or an amino acid sequence sharing at least 80, 90%, 95% or 98% identity therewith) and the light chain variable region comprises an amino acid sequence of the NKp46-4 L2 variable domain (or an amino acid sequence sharing at least 80, 90%, 95% or 98% identity therewith).

In one aspect, provided is a protein or polypeptide (e.g. a monospecific, bispecific, or multispecific antibody or protein, a scFv, a F(ab) or F(ab)₂, a multispecific Fc protein) that binds a human NKp46 polypeptide, optionally that further bind a non-human primate NKp46 polypeptide, wherein the protein nor polypeptide comprises: (a) a heavy chain variable region comprising a human heavy chain framework region (FR1, FR2, FR3) derived from a IGHV4-30-4 gene, and heavy chain CDR1, 2 and 3 of a NKp46-9 antibody; and (b) a light chain variable region comprising a human light chain framework region (FR1, FR2, FR3) derived from a IGKV1-39 gene, and light chain CDR1, 2 and 3 of a NKp46-9 antibody. Optionally, CDRs as defined by Kabat, Chotia or IMGT numbering. In one embodiment, the heavy chain variable region comprises an amino acid sequence sharing at least 70%, 80%, 90%, 95% or 98% identity with the amino acid sequence of SEQ ID NO: 13, and/or the light chain variable region comprises an amino acid sequence sharing at least 70%, 80%, 90%, 95% or 98% identity with the amino acid sequence of SEQ ID NO: 14. In one embodiment, the heavy chain variable region comprises an amino acid sequence of the NKp46-9 H2 variable domain (or an amino acid sequence sharing at least 80, 90%, 95% or 98% identity therewith) and the light chain variable region comprises an amino acid sequence of the NKp46-9 L1 or L2 variable domain (or an amino acid sequence sharing at least 80, 90%, 95% or 98% identity therewith). In one embodiment, the heavy chain variable region comprises an amino acid sequence of the NKp46-9 H3 variable domain (or an amino acid sequence sharing at least 80, 90%, 95% or 98% identity therewith) and the light chain variable region comprises an amino acid sequence of the NKp46-9 L1 or L2 variable domain (or an amino acid sequence sharing at least 80, 90%, 95% or 98% identity therewith). In one aspect, provided is an antigen binding domain comprising a heavy chain variable region (VH) and a light chain variable region (VL) each containing human FR1, 2 and 3 framework regions, or a protein or polypeptide comprising such ABD (e.g. a monoclonal antibody, an scFv, a multispecific polypeptide, a bispecific antibody, a DART® or BiTe® or protein comprising such, etc.) that binds a human and a non-human primate NKp46 polypeptide, e.g. a cell surface NKp46 polypeptide, wherein the protein or polypeptide comprises a VH and VL combination selected from the group consisting of:
(a) a VH comprising an amino acid sequence at least 70%, 80%, 90%, 95% or 98% (or 100%) identical to the amino acid sequence of the NKp46-1 H1 or H3 variable domain shown in SEQ ID NOS: 199 or 200, respectively, and a VL comprising an amino acid sequence at least 70%, 80%, 90%, 95% or 98% identical to the amino acid sequence of the NKp46-1 L1 variable domain shown in SEQ ID NO: 201;
(b) a VH comprising an amino acid sequence at least 70%, 80%, 90%, 95% or 98% (or 100%) identical to the amino acid sequence of the NKp46-2 H1, H2 or H3 variable domain shown in SEQ ID NOS: 202, 203 or 203, respectively, and a VL comprising an amino acid sequence at least 70%, 80%, 90%, 95% or 98% identical to the amino acid sequence of the NKp46-2 L1 variable domain shown in SEQ ID NO: 205;
(c) a VH comprising an amino acid sequence at least 70%, 80%, 90%, 95% or 98% (or 100%) identical to the amino acid sequence of the NKp46-3 H1, H3 or H4 variable domain shown in SEQ ID NOS: 206, 207 or 208, respectively, and a VL comprising an amino acid sequence at least 70%, 80%, 90%, 95% or 98% identical to the amino acid sequence of the NKp46-3 L1 variable domain shown in SEQ ID NO: 209;
(d) a VH comprising an amino acid sequence at least 70%, 80%, 90%, 95% or 98% (or 100%) identical to the amino acid sequence of the NKp46-4 H1, H2 or H3 variable domain shown in SEQ ID NOS: 210, 211 or 212, and a VL comprising an amino acid sequence at least 70%, 80%, 90%, 95% or 98% identical to the amino acid sequence of the NKp46-4 L2 variable domain shown in SEQ ID NO: 213;
(e) a VH comprising an amino acid sequence at least 70%, 80%, 90%, 95% or 98% (or 100%) identical to the amino acid sequence of the NKp46-9 H2 variable domain shown in SEQ ID NO: 215, and a VL comprising an amino acid sequence at least 70%, 80%, 90%, 95% or 98% identical to the amino acid sequence of the NKp46-9 L1 or L2 variable domain shown in SEQ ID NOS: 217 or 218, respectively; or
(f) a VH comprising an amino acid sequence at least 70%, 80%, 90%, 95% or 98% (or 100%) identical to the amino acid sequence of the NKp46-9 H3 variable domain shown in SEQ ID NO: 215, and a VL comprising an amino acid sequence at least 70%, 80%, 90%, 95% or 98% identical to the amino acid sequence of the NKp46-9 L1 or L2 variable domain shown in SEQ ID NOS: 217 or 218, respectively.

In one aspect, provided is an antigen binding domain comprising a heavy chain variable region (VH) and a light chain variable region (VL) each containing human FR1, 2 and 3 framework regions, or a protein or polypeptide comprising such ABD that binds a human and a non-human primate NKp46 polypeptide, wherein the protein or polypeptide comprises a VH and VL combination selected from the group consisting of:
(a) a VH comprising a CDR1, 2 and 3 of the VH domain having the amino acid sequence shown in SEQ ID NO: 3 and a FR1, 2 and 3 of a human IGHV1-69 gene segment, and a VL comprising a CDR1, 2 and 3 of the VL domain having the amino acid sequence shown in SEQ ID NO: 4 and a FR1, 2 and 3 of a human IGKV1-33 gene segment;
(b) a VH comprising a CDR1, 2 and 3 of the VH domain having the amino acid sequence shown in SEQ ID NO: 5 and a FR1, 2 and 3 of a human IGHV4-30-4 gene segment, and a VL comprising a CDR1, 2 and 3 of the VL domain having the amino acid sequence shown in SEQ ID NO: 6 and a FR1, 2 and 3 of a human IGKV1-39 gene segment;
(c) a VH comprising a CDR1, 2 and 3 of the VH domain having the amino acid sequence shown in SEQ ID NO: 7 and a FR1, 2 and 3 of a human IGHV1-69 gene segment, and a VL comprising a CDR1, 2 and 3 of the VL domain having the amino acid sequence shown in SEQ ID NO: 8 and a FR1, 2 and 3 of a human IGKV3-11 and/or IGKV3-15 gene segment;
(d) a VH comprising a CDR1, 2 and 3 of the VH domain having the amino acid sequence shown in SEQ ID NO: 9 and a FR1, 2 and 3 of a human IGHV1-46 and/or a IGHV1-69 gene segment, and a VL comprising a CDR1, 2 and 3 of the VL domain having the amino acid sequence shown in SEQ ID NO: 10 and a FR1, 2 and 3 of a human IGKV1-NL1 gene segment;
(e) a VH comprising a CDR1, 2 and 3 of the VH domain having the amino acid sequence shown in SEQ ID NO: 13 and a FR1, 2 and 3 of a human IGHV4-30-4 gene segment, and a VL comprising a CDR1, 2 and 3 of the VL domain having the amino acid sequence shown in SEQ ID NO: 14 and a FR1, 2 and 3 of a human IGKV1-39 gene segment.

In any aspect, an antigen binding domain may comprise a VH and a VL comprising an amino acid sequence at least 80%, 90%, 95% or 98% (or 100%) identical to the respective VH and VL of any one of antibodies: NKp46-1 H1L1, NKp46-1 H3L1, NKp46-2 H1L1, NKp46-2 H2L1, NKp46-2 H3L1, NKp46-3 H1L1, NKp46-3 H3L1, NKp46-3 H4L1, NKp46-4 H1L2, NKp46-4 H2L2, NKp46-4 H3L2, NKp46-9 H2L1, NKp46-9 H2L2, NKp46-9 H3L1 or NKp46-9 H3L2.

In one aspect, provided is an antigen binding domain comprising a heavy chain variable region (VH) and a light chain variable region (VL) each containing human FR1, 2 and 3 framework regions, or a protein or polypeptide (e.g., an antibody, multispecific protein) comprising such ABD that binds a NKp46 polypeptide, wherein the protein or polypeptide comprising an amino acid sequence at least 80%, 90%, 95% or 98% (or 100%) identical to the respective VH and VL of any one of antibodies: NKp46-1 H1L1, NKp46-1 H3L1, NKp46-2 H1L1, NKp46-2 H2L1, NKp46-2 H3L1, NKp46-3 H1L1, NKp46-3 H3L1, NKp46-3 H4L1, NKp46-4 H1L2, NKp46-4 H2L2, NKp46-4 H3L2, NKp46-9 H2L1, NKp46-9 H2L2, NKp46-9 H3L1 or NKp46-9 H3L2. VH and VL sequences of these antibodies are listed in Table C and Example 1 part B.

In one embodiment, a NKp46-1 VL can comprising an amino acid substitution at Kabat position 87, optionally wherein the residue at position 87 is a phenylalanine. A NKp46-1 VH can comprise an amino acid substitution at Kabat residues 27, 66 and/or 67, optionally further a substitution at Kabat residues 37, 48 and/or 91. Optionally the residue present at the particular position is the residue substituted at the position herein, e.g., as shown in Example 1.

In one embodiment, a NKp46-2 VL can comprise an amino acid substitution at Kabat position 48, optionally wherein the residue at position 48 is a valine. A NKp46-2 VH can comprise an amino acid substitution at Kabat residues 27 and/or 71, optionally further a substitution at Kabat residues 48 and/or 67, optionally further a substitution at Kabat residue 31. Optionally the residue present at the particular position is the residue substituted at the position herein, e.g., as shown in Example 1.

In one embodiment, a NKp46-3 VL can comprise an amino acid substitution at Kabat position 49, optionally wherein the residue at position 49 is a lysine. A NKp46-3 VH can comprise an amino acid substitution at Kabat residue 27, optionally further a substitution at Kabat residues 48 and/or 67, optionally further a substitution at Kabat residue 69. Optionally the residue present at the particular position is the residue substituted at the position herein, e.g., as shown in Example 1.

In one embodiment, a NKp46-4 VL can comprise an amino acid substitution at Kabat position 36 and/or 48, optionally wherein the residue at position 36 is a phenylalanine, optionally wherein the residue at position 48 is a valine. A NKp46-4 VH can comprise an amino acid substitution at Kabat residues 30, 48 and/or 93, optionally further a substitution at Kabat residue 67, optionally further a substitution at Kabat residue 69. Optionally the residue present at the particular position is the residue substituted at the position herein, e.g., as shown in Example 1.

In one embodiment, a NKp46-9 VL can comprise an amino acid substitution at Kabat position 36, optionally wherein the residue at position 36 is a cysteine, optionally further a substitution at Kabat residue 48, optionally wherein the residue at position 48 is a valine. A NKp46-9 VH can comprise an amino acid substitution at Kabat residue 71, optionally further a substitution at Kabat residue 27, optionally further a substitution at Kabat residue 48 and/or 67. Optionally the residue present at the particular position is the residue substituted at the position herein, e.g., as shown in Example 1.

In one aspect of any embodiment, the protein or polypeptide binds NKp46 in monovalent manner. In aspect of any embodiment, the protein or polypeptide comprises or contains no more than one antigen binding domain that binds NKp46.

In one embodiment, the protein or polypeptide comprises a human Fc domain or a portion thereof, wherein the Fc domain or portion thereof is capable of binding to human neonatal Fc receptor (FcRn) and to human CD16 polypeptide. Because FcγRIIIa (CD16) is not present on all NK cells, conventional therapeutic antibodies (e.g. of human isotypes IgG1) designed to exert antibody-dependent cellular toxicity (ADCC) via FcγRIIIa may not mobilize all NK cells; the present proteins on the other hand enable all NK cells to be solicited via NKp46, and the proteins will thus be useful to activate or increase the cytolytic activity of NKp46⁺CD16⁻ NK cells as well as NKp46⁺CD16⁺ NK cells.

In one embodiment, the protein or polypeptide comprises a human Fc domain or a portion thereof, wherein the Fc domain or a portion thereof is capable of binding to human neonatal Fc receptor (FcRn) and lacks binding to human CD16 polypeptide.

In one embodiment, the protein or polypeptide is a single chain polypeptide. In one embodiment, the protein or polypeptide is a multimeric polypeptide, optionally a dimeric, trimeric or tetrameric protein.

Advantageously, in one embodiment, the presence of NK cells and target cells, a multi-specific protein can bind (i) to antigen of interest on target cells and (ii) to NKp46 on NK cells, and, when bound to both antigen of interest on target cells and NKp46, can induce signaling in and/or activation of the NK cells through NKp46 (the protein acts as an NKp46 agonist), thereby promoting activation of NK cells and/or lysis of target cells, notably via the activating signal transmitted by NKp46. In specific advantageous embodiments, the multi-specific comprises a single antigen binding domain that binds NKp46 (the protein binds to NKp46 in monovalent fashion). In one embodiment the protein is capable of, when bound to both antigen of interest on target cells and NKp46 on NK cells, inducing signaling in the NK cells through NKp46. In one embodiment, the protein comprises a first antigen binding domain and a second antigen binding domain, wherein one of the first or second antigen binding domains binds to a human NKp46 polypeptide and the other of the first or second antigen binding domains binds an antigen of interest expressed on a target cell.

In one embodiment, a multispecific protein is monomeric. In one embodiment a multispecific protein is a dimer, e.g. a heterodimer, trimer or tetramer. In one embodiment, the protein is a heterodimer, heterotrimer or a tetramer comprising a first polypeptide chain comprising a VH and a VL domain of an ABD that binds NKp46 (a VH and VL disclosed herein) separated by a linker peptide, and optionally an Fc domain, and a second polypeptide chain comprising one or more variable regions or antigen binding domains that bind an antigen of interest, and optionally an Fc domain. In one embodiment, both a first and a second polypeptide chain comprise an Fc domain, and the protein comprises a dimeric Fc domain, optionally wherein the Fc domain is capable of binding to human CD16. In one embodiment, the monomeric or dimeric protein comprises a protein with a domain structure in which an Fc domain is interposed between the antigen binding domain (ABD) that binds to NKp46 and the antigen binding domain that binds an antigen of interest. In one embodiment the multispecific Fc-derived polypeptide is a bispecific antibody.

In one embodiment of any of the protein herein, the antigen binding domain of a multispecific protein that binds to an antigen of interest binds to an antigen (e.g. polypeptide) expressed by a target cell which sought to be lysed by an NK cell. Optionally such an antigen is expressed by a cancer cell, a tumor associated immune cell (e.g. an immune suppressor or regulatory cell), a virally infected cell, or a cell that contributes to an autoimmunity or inflammatory disease. Optionally, the antigen of interest is a cancer antigen known to be capable of undergoing intracellular internalization when contacted with a full length human IgG1 antibody that binds specifically thereto.

In one aspect of any of the embodiments herein, provided is a recombinant nucleic acid encoding a VH and/or a VL, an antibody heavy and/or light chain, a single chain antigen binding domain, a first, second, third or further polypeptide chain of any of the proteins of the disclosure. In one aspect of any of the embodiments herein, provided is a recombinant host cell comprising a nucleic acid of the disclosure.

Any of the methods can further be characterized as comprising any step described in the application, including notably in the “Detailed Description of the Invention”). The invention further relates to methods of identifying, testing and/or making proteins described herein. The invention further relates to a multispecific protein obtainable by any of present methods. The disclosure further relates to pharmaceutical or diagnostic formulations of the multispecific protein disclosed herein. The disclosure further relates to methods of using the multispecific protein in methods of treatment or diagnosis.

These and additional advantageous aspects and features of the invention may be further described elsewhere herein.

### BRIEF DESCRIPTION OF THE FIGURES

FIG. 1 shows that Anti-CD19-F1-Anti-CD3 does not cause T/B cell aggregation in the presence of B221 (CD19) or JURKAT (CD3) cell lines when separate, but it does cause aggregation of cells when both B221 and JURKAT cells are co-incubated.

FIGS. 2A to 2F show different domain arrangements of bispecific proteins produced.

FIGS. 3A and 3B show respectively bispecific F1 and F2 antibodies having NKp46 binding region based on NKp46-1, NKp46-2, NKp46-3 or NKp46-4 are able to direct resting NK cells to their CD19-positive Daudi tumor target cells, while isotype control antibody did not lead to elimination of the Daudi cells. Rituximab (RTX) served as positive control of ADCC, where the maximal response obtained with RTX (at 10 μg/ml in this assay) was 21.6% specific lysis.

FIG. 4A (top panel CD107, bottom panel CD69) shows bispecific antibodies having NKp46 and CD19 binding regions in an F2 format protein do not activate resting NK cells in the absence of target cells, however full length anti-NKp46 antibodies as well as positive control alemtuzumab did activate NK cells. FIG. 4B shows that in presence of Daudi target cells, bispecific anti-NKp46 x anti-CD19 antibodies (including each of the NKp46-1, NKp46-2, NKp46-3 or NKp46-4 binding domains) activated resting NK cells (top panel CD107, bottom panel CD69), while full-length anti-CD19 showed at best only very low activation of NK cells. Neither full-length anti-NKp46 antibodies or alemtuzumab showed substantial increase in activation beyond what was observed in presence of NK cells alone. FIG. 4C (top panel CD107, bottom panel CD69) shows that in the presence of CD19-negative HUT78 cells, none of the bispecific anti-NKp46 x anti-CD19 antibody (including each of the NKp46-1, NKp46-2, NKp46-3 or NKp46-4 variable regions) activated NK cells. However, the full-length anti-NKp46 antibodies and alemtuzumab caused detectable activation of NK cells at a similar level observed in presence of NK cells alone. Isotype control antibody did not induce activation.

FIGS. 5A and 5B show that at low effector:target ratio of 1:1 each of the bispecific anti-NKp46 x anti-CD19 antibody activated NK cells in the presence of Daudi cells, and that bispecific anti-NKp46 x anti-CD19 were far more potent than the anti-CD19 antibody as a full-length human IgG1 as ADCC inducing antibody. Top panel is CD107 (FIG. 5A) and bottom panel shows CD69 (FIG. 5B).

FIGS. 6A and 6B show that each NKp46 x CD19 bispecific protein (single chain format F3, and multimeric formats F5 and F6) induced specific lysis of Daudi (FIG. 6A) or B221 (FIG. 6B) cells by human KHYG-1 CD16-negative hNKp46-positive NK cell line, while rituximab and human IgG1 isotype control (IC) antibodies did not.

FIG. 7 shows the NKp46 x CD19 bispecific protein in F5 format whose Fc domain binds CD16 is far more potent in mediating Daudi target cell lysis that the full-length IgG1 anti-CD19 antibody or the F6 format bispecific protein. The bispecific anti-CD19 in F6 format whose Fc domain does not bind CD16 was as potent in mediating NK cell lysis of Daudi target cells as the full-length IgG1 anti-CD19 antibody, which is remarkable considering that the control IgG1 anti-CD19 antibody binds CD19 bivalently.

### DETAILED DESCRIPTION OF THE INVENTION

As used in the specification, “a” or “an” may mean one or more. As used in the claim(s), when used in conjunction with the word “comprising”, the words “a” or “an” may mean one or more than one.

Where “comprising” is used, this can optionally be replaced by “consisting essentially of”, more optionally by “consisting of”.

As used herein, the term “antigen binding domain” refers to a domain comprising a three-dimensional structure capable of immunospecifically binding to an epitope. Thus, in one embodiment, said domain can comprise a hypervariable region, optionally a VH and/or VL domain of an antibody chain, optionally at least a VH domain. In another embodiment, the binding domain may comprise at least one complementarity determining region (CDR) of an antibody chain. In another embodiment, the binding domain may comprise a polypeptide domain from a non-immunoglobulin scaffold.

The term “antibody” herein is used in the broadest sense and specifically includes full-length monoclonal antibodies, polyclonal antibodies, multispecific antibodies (e.g., bispecific antibodies), and antibody fragments and derivatives, so long as they exhibit the desired biological activity. Various techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988). An “antibody fragment” comprises a portion of a full-length antibody, e.g. antigen-binding or variable regions thereof. Examples of antibody fragments include Fab, Fab′, F(ab)₂, F(ab′)₂, F(ab)₃, Fv (typically the VL and VH domains of a single arm of an antibody), single-chain Fv (scFv), dsFv, Fd fragments (typically the VH and CH1 domain), and dAb (typically a VH domain) fragments; VH, VL, VhH, and V-NAR domains; minibodies, diabodies, triabodies, tetrabodies, and kappa bodies (see, e.g., III et al., Protein Eng 1997;10: 949-57); camel IgG; IgNAR; and multispecific antibody fragments formed from antibody fragments, and one or more isolated CDRs or a functional paratope, where isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23, 1126-1136; WO2005040219, and published U.S. Patent Applications 20050238646 and 20020161201.

The term “antibody derivative”, as used herein, comprises a full-length antibody or a fragment of an antibody, e.g. comprising at least antigen-binding or variable regions thereof, wherein one or more of the amino acids are chemically modified, e.g., by alkylation, PEGylation, acylation, ester formation or amide formation or the like. This includes, but is not limited to, PEGylated antibodies, cysteine-PEGylated antibodies, and variants thereof.

The term “hypervariable region” when used herein refers to the amino acid residues of an antibody that are responsible for antigen binding. The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987;196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

By “constant region” as defined herein is meant an antibody-derived constant region that is encoded by one of the light or heavy chain immunoglobulin constant region genes. By “constant light chain” or “light chain constant region” as used herein is meant the region of an antibody encoded by the kappa (Ckappa) or lambda (Clambda) light chains. The constant light chain typically comprises a single domain, and as defined herein refers to positions 108-214 of Ckappa, or Clambda, wherein numbering is according to the EU index (Kabat et al., 1991, Sequences of Proteins of Immunological Interest, 5th Ed., United States Public Health Service, National Institutes of Health, Bethesda). By “constant heavy chain” or “heavy chain constant region” as used herein is meant the region of an antibody encoded by the mu, delta, gamma, alpha, or epsilon genes to define the antibody's isotype as IgM, IgD, IgG, IgA, or IgE, respectively. For full length IgG antibodies, the constant heavy chain, as defined herein, refers to the N-terminus of the CH1 domain to the C-terminus of the CH3 domain, thus comprising positions 118-447, wherein numbering is according to the EU index.

By “Fab” or “Fab region” as used herein is meant the polypeptide that comprises the VH, CH1, VL, and CL immunoglobulin domains. Fab may refer to this region in isolation, or this region in the context of a polypeptide, multispecific polypeptide or ABD, or any other embodiments as outlined herein.

By “single-chain Fv” or “scFv” as used herein are meant antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding. Methods for producing scFvs are well known in the art. For a review of methods for producing scFvs see Pluckthun in The Pharmacology of Monoclonal Antibodies, vol. 113, Rosenburg and Moore eds. Springer-Verlag, New York, pp. 269-315 (1994).

By “Fv” or “Fv fragment” or “Fv region” as used herein is meant a polypeptide that comprises the VL and VH domains of a single antibody.

By “Fc” or “Fc region”, as used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 (CH2) and Cγ3 (CH3) and the hinge between Cγ1 and Cγ2. Although the boundaries of the Fc region may vary, the human IgG heavy chain Fc region is usually defined to comprise residues at least C226, P230 or A231 to its carboxyl-terminus, wherein the numbering is according to the EU index. Fc may refer to this region in isolation, or this region in the context of an Fc polypeptide, as described below. By “Fc polypeptide” or “Fc-derived polypeptide” as used herein is meant a polypeptide that comprises all or part of an Fc region. Fc polypeptides include but is not limited to antibodies, Fc fusions and Fc fragments.

By “variable region” as used herein is meant the region of an antibody that comprises one or more Ig domains substantially encoded by any of the VL (including Vkappa (VK) and Vlambda) and/or VH genes that make up the light chain (including kappa and lambda) and heavy chain immunoglobulin genetic loci respectively. A light or heavy chain variable region (VL or VH) consists of a “framework” or “FR” region interrupted by three hypervariable regions referred to as “complementarity determining regions” or “CDRs”. The extent of the framework region and CDRs have been precisely defined, for example as in Kabat (see “Sequences of Proteins of Immunological Interest,” E. Kabat et al., U.S. Department of Health and Human Services, (1983)), and as in Chothia. The framework regions of an antibody, that is the combined framework regions of the constituent light and heavy chains, serves to position and align the CDRs, which are primarily responsible for binding to an antigen.

The term “specifically binds to” means that an antibody or polypeptide can bind preferably in a competitive binding assay to the binding partner, e.g. NKp46, as assessed using either recombinant forms of the proteins, epitopes therein, or native proteins present on the surface of isolated target cells. Competitive binding assays and other methods for determining specific binding are further described below and are well known in the art.

When an antibody or polypeptide is said to “compete with” a particular monoclonal antibody (e.g. NKp46-1, -2, -4, -6 or -9 in the context of an anti-NKp46 mono- or bi-specific antibody), it means that the antibody or polypeptide competes with the monoclonal antibody in a binding assay using either recombinant target (e.g. NKp46) molecules or surface expressed target (e.g. NKp46) molecules. For example, if a test antibody reduces the binding of NKp46-1, -2, -4, -6 or -9 to a NKp46 polypeptide or NKp46-expressing cell in a binding assay, the antibody is said to “compete” respectively with NKp46-1, -2, -4, -6 or -9.

The term “affinity”, as used herein, means the strength of the binding of an antibody or polypeptide to an epitope. The affinity of an antibody is given by the dissociation constant K_{D}, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant K_{A} is defined by 1/K_{D}. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of surface plasmon resonance (SPR) screening (such as by analysis with a BIAcore™ SPR analytical device).

Within the context of this invention a “determinant” designates a site of interaction or binding on a polypeptide.

The term “epitope” refers to an antigenic determinant, and is the area or region on an antigen to which an antibody or polypeptide binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

By “amino acid modification” herein is meant an amino acid substitution, insertion, and/or deletion in a polypeptide sequence. An example of amino acid modification herein is a substitution. By “amino acid modification” herein is meant an amino acid substitution, insertion, and/or deletion in a polypeptide sequence. By “amino acid substitution” or “substitution” herein is meant the replacement of an amino acid at a given position in a protein sequence with another amino acid. For example, the substitution Y50W refers to a variant of a parent polypeptide, in which the tyrosine at position 50 is replaced with tryptophan. A “variant” of a polypeptide refers to a polypeptide having an amino acid sequence that is substantially identical to a reference polypeptide, typically a native or “parent” polypeptide. The polypeptide variant may possess one or more amino acid substitutions, deletions, and/or insertions at certain positions within the native amino acid sequence.

“Conservative” amino acid substitutions are those in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

An “isolated” molecule is a molecule that is the predominant species in the composition wherein it is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of molecule, e.g., peptide, in the composition). Commonly, a composition of a polypeptide will exhibit 98%, 98%, or 99% homogeneity for polypeptides in the context of all present peptide species in the composition or at least with respect to substantially active peptide species in the context of proposed use.

In the context herein, “treatment” or “treating” refers to preventing, alleviating, managing, curing or reducing one or more symptoms or clinically relevant manifestations of a disease or disorder, unless contradicted by context. For example, “treatment” of a patient in whom no symptoms or clinically relevant manifestations of a disease or disorder have been identified is preventive or prophylactic therapy, whereas “treatment” of a patient in whom symptoms or clinically relevant manifestations of a disease or disorder have been identified generally does not constitute preventive or prophylactic therapy.

As used herein, “NK cells” refers to a sub-population of lymphocytes that is involved in non-conventional immunity. NK cells can be identified by virtue of certain characteristics and biological properties, such as the expression of specific surface antigens including CD56 and/or NKp46 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Any subpopulation of NK cells will also be encompassed by the term NK cells. Within the context herein “active” NK cells designate biologically active NK cells, including NK cells having the capacity of lysing target cells or enhancing the immune function of other cells. NK cells can be obtained by various techniques known in the art, such as isolation from blood samples, cytapheresis, tissue or cell collections, etc. Useful protocols for assays involving NK cells can be found in Natural Killer Cells Protocols (edited by Campbell KS and Colonna M). Human Press. pp. 219-238 (2000).

The term “internalization”, used interchangeably with “intracellular internalization”, refers to the molecular, biochemical and cellular events associated with the process of translocating a molecule from the extracellular surface of a cell to the intracellular surface of a cell. The processes responsible for intracellular internalization of molecules are well-known and can involve, inter alia, the internalization of extracellular molecules (such as hormones, antibodies, and small organic molecules); membrane-associated molecules (such as cell-surface receptors); and complexes of membrane-associated molecules bound to extracellular molecules (for example, a ligand bound to a transmembrane receptor or an antibody bound to a membrane-associated molecule). Thus, “inducing and/or increasing internalization” comprises events wherein intracellular internalization is initiated and/or the rate and/or extent of intracellular internalization is increased.

As used herein, an agent that has “agonist” activity at Nkp46 is an agent that can cause or increase “NKp46 signaling”. “Nkp46 signaling” refers to an ability of a NKp46 polypeptide to activate or transduce an intracellular signaling pathway. Changes in NKp46 signaling activity can be measured, for example, by assays designed to measure changes in NKp46 signaling pathways, e.g. by monitoring phosphorylation of signal transduction components, assays to measure the association of certain signal transduction components with other proteins or intracellular structures, or in the biochemical activity of components such as kinases, or assays designed to measure expression of reporter genes under control of NKp46-sensitive promoters and enhancers, or indirectly by a downstream effect mediated by the NKp46 polypeptide (e.g. activation of specific cytolytic machinery in NK cells). Reporter genes can be naturally occurring genes (e.g. monitoring cytokine production) or they can be genes artificially introduced into a cell. Other genes can be placed under the control of such regulatory elements and thus serve to report the level of NKp46 signaling.

“NKp46” refers to a protein or polypeptide encoded by the Ncr1 gene or by a cDNA prepared from such a gene. Any naturally occurring isoform, allele or variant is encompassed by the term NKp46 polypeptide (e.g., an NKp46 polypeptide 90%, 95%, 98% or 99% identical to SEQ ID NO 1, or a contiguous sequence of at least 20, 30, 50, 100 or 200 amino acid residues thereof). The 304 amino acid residue sequence of human NKp46 (isoform a) is shown as follows:

(SEQ ID NO: 1)
MSSTLPALLC VGLCLSQRIS AQQQTLPKPF IWAEPHFMVP
KEKQVTICCQ GNYGAVEYQL HFEGSLFAVD RPKPPERINK
VKFYIPDMNS RMAGQYSCIY RVGELWSEPS NLLDLVVTEM
YDTPTLSVHP GPEVISGEKV TFYCRLDTAT SMFLLLKEGR
SSHVQRGYGK VQAEFPLGPV TTAHRGTYRC FGSYNNHAWS
FPSEPVKLLV TGDIENTSLA PEDPTFPADT WGTYLLTTET
GLQKDHALWD HTAQNLLRMG LAFLVLVALV WFLVEDWLSR
KRTRERASRA STWEGRRRLN TQTL.

SEQ ID NO: 1 corresponds to NCBI accession number NP_004820, the disclosure of which is incorporated herein by reference. The human NKp46 mRNA sequence is described in NCBI accession number NM_004829, the disclosure of which is incorporated herein by reference. The amino acid residue sequence of the extracellular domain of the macaca fascicularis (cynomolgus) NKp46 polypeptide is shown as follows:

MSSTLRALLCLGLCLSQRISAPKQTLPKPIIRAESTYMVPKEKQA
TLCCQGSYGAVEYQLHFEGSLFAVERPKPPERINGVKFHIPDMNSRKAGR
YSCIYRVGELWSERSDLLDLVVTEMYDTPTLSVHPGPEVTSGEKVTFYCR
LDTATSMFLLLKEGRSRDVQRSYGKVQAEFPMGPVTTAHRGSYRCFGSYN
NYAWSFPSEPVKLLVTGDIENTSLAPTDPTFPDSWDTCLLTRETGLQKDL
ALWDHTAQN.

### Producing Anti-NKp46 Antibodies

The anti-NKp46 antigen binding domains bind an extra-cellular portion of human NKp46 polypeptide. In one aspect, the protein that comprises a VH and VL of the disclosure is a humanized antibody or comprises an antigen binding fragment thereof. In one aspect, the antibody comprises a constant domain selected from a human IgG1, IgG2, IgG3 and IgG4 constant domain, optionally comprising one or more amino acid modifications. In one aspect, anti-NKp46 antibody comprises an antibody fragment selected from a Fab fragment, a Fab′ fragment, a Fab′-SH fragment, a F(ab)2 fragment, a F(ab′)2 fragment, an Fv fragment, a Heavy chain Ig (a llama or camel Ig), a VHH fragment, a single domain FV, and a single-chain antibody fragment. In one aspect, the anti-NKp46 antibody comprises a synthetic or semisynthetic antibody-derived molecule selected from a scFV, a dsFV, a minibody, a diabody, a triabody, a kappa body, an IgNAR; and a multispecific antibody. The agent can optionally further comprise an Fc domain. In one aspect, the antibody is in at least partially purified form. In one aspect, the antibody is in essentially isolated form.

Antibodies may be produced by a variety of techniques known in the art. Immunological screening assays in which antibody competition can be assessed can be used to select for antibodies that will bind the same epitope on NKp46 as antigen binding domains herein can be assessed, e.g. as described in for example, in PCT application number PCT/EP2016/064537, filed 23 Jun. 2016 (Innate Pharma), the disclosure of which is incorporated herein by reference.

Typically, an anti-NKp46 antibody or NKp46 binding protein provided herein has an affinity for a NKp46 polypeptide (e.g., a NKp46 polypeptide as produced in the Examples herein) in the range of about 10⁴ to about 10¹¹ M⁻¹ (e.g., about 10⁸ to about 10¹⁰ M⁻¹). For example, in a particular aspect the disclosure provides Anti-NKp46 antibody or NKp46 binding protein that has an average disassociation constant (K_{D}) of less than 1×10⁻⁹ M with respect to NKp46, as determined by, e.g., surface plasmon resonance (SPR) screening (such as by analysis with a BIAcore™ SPR analytical device). In a more particular exemplary aspect, the disclosure provides anti-NKp46 antibodies or NKp46 binding proteins that have a KD of about 1×10⁻⁸ M to about 1×10⁻¹⁰ M, or about 1×10⁻⁹ M to about 1×10⁻¹¹ M, for human or cynomolgus NKp46 protein. In one embodiment, the NKp46 antibodies or NKp46 binding proteins have a disassociation constant (K_{D}) for human and cynomolgus NKp46 protein that differs by no more than 1- or 2-log.

Antibodies or NKp46 binding proteins can be characterized for example by a mean KD of no more than about (i.e. better affinity than) 100, 60, 10, 5, or 1 nanomolar, preferably sub-nanomolar or optionally no more than about 500, 200, 100 or 10 picomolar. KD can be determined for example for example by immobilizing recombinantly produced human NKp46 proteins on a chip surface, followed by application of the antibody to be tested in solution.

DNA encoding an antibody or other NKp46 binding proteins that binds an epitope present on NKp46 polypeptides is isolated and placed in an appropriate expression vector for transfection into an appropriate host. The host is then used for the recombinant production of the antibody or other NKp46 binding proteins. For example, DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of the desired proteins in the recombinant host cells.

In one embodiment, the proteins and antibodies herein bind the D1 domain of NKp46, the D2 domain of NKp46, or to a region spanning both the D1 and D2 domains (at the border of the D1 and D2 domains, the D1/D2 junction), of the NKp46 polypeptide of SEQ ID NO: 1. In one embodiment, the proteins or antibodies have an affinity for human NKp46 characterized by a K_{D} of less than 10⁻⁸ M, less than 10⁻⁹ M, or less than 10⁻¹⁰M.

In another embodiment, the proteins or antibodies bind NKp46 at substantially the same epitope on NKp46 as antibody NKp46-1, NKp46-2, NKp46-3 or NKp46-4. In another embodiment, the antibodies at least partially overlaps, or includes at least one residue in the segment bound by NKp46-1, NKp46-2, NKp46-3 or NKp46-4. In one embodiment, all key residues of the epitope are in a segment corresponding to domain D1 or D2. In one embodiment, the antibody binds a residue present in the D1 domain as well as a residue present in in the D2 domain. In one embodiment, the antibodies or proteins bind an epitope comprising 1, 2, 3, 4, 5, 6, 7 or more residues in the segment corresponding to domain D1 or D2 of the NKp46 polypeptide of SEQ ID NO: 1. In one embodiment, the antibodies or proteins bind domain D1 and bind an epitope comprising 1, 2, 3, or 4 of the residues R101, V102, E104 and/or L105. In one embodiment, the antibodies or proteins bind domain D1/D2 junction and bind an epitope comprising 1, 2, 3, 4 or 5 of the residues K41, E42, E119, Y121 and/or Y194. In one embodiment, the antibodies bind domain D2 and bind an epitope comprising 1, 2, 3, or 4 of the residues P132, E133, 1135, and/or S136.

The Examples section herein describes the construction of a series of mutant human NKp46 polypeptides. Binding of anti-NKp46 antibody or proteins to cells transfected with the NKp46 mutants was measured and compared to the ability of anti-NKp46 antibody or protein to bind wild-type NKp46 polypeptide (SEQ ID NO:1). A reduction in binding between an anti-NKp46 antibody or protein and a mutant NKp46 polypeptide as used herein means that there is a reduction in binding affinity (e.g., as measured by known methods such FACS testing of cells expressing a particular mutant, or by Biacore testing of binding to mutant polypeptides) and/or a reduction in the total binding capacity of the anti-NKp46 antibody or protein (e.g., as evidenced by a decrease in Bmax in a plot of anti-NKp46 antibody concentration versus polypeptide concentration). A significant reduction in binding indicates that the mutated residue is directly involved in binding to the anti-NKp46 antibody or protein or is in close proximity to the binding protein when the anti-NKp46 antibody or protein is bound to NKp46. An epitope will thus preferably include such residue and may include additional residues adjacent to such residue.

In some embodiments, a significant reduction in binding means that the binding affinity and/or capacity between an anti-NKp46 antibody or protein and a mutant NKp46 polypeptide is reduced by greater than 40%, greater than 50%, greater than 55%, greater than 60%, greater than 65%, greater than 70%, greater than 75%, greater than 80%, greater than 85%, greater than 90% or greater than 95% relative to binding between the antibody or protein and a wild type NKp46 polypeptide (e.g., the polypeptide shown in SEQ ID NO:1). In certain embodiments, binding is reduced below detectable limits. In some embodiments, a significant reduction in binding is evidenced when binding of an anti-NKp46 antibody to a mutant NKp46 polypeptide is less than 50% (e.g., less than 45%, 40%, 35%, 30%, 25%, 20%, 15% or 10%) of the binding observed between the anti-NKp46 antibody and a wild-type NKp46 polypeptide (e.g., the polypeptide shown in SEQ ID NO: 1 (or the extracellular domain thereof)). Such binding measurements can be made using a variety of binding assays known in the art. A specific example of one such assay is described in the Example section.

In some embodiments, anti-NKp46 antibodies or proteins are provided that exhibit significantly lower binding for a mutant NKp46 polypeptide in which a residue in a wild-type NKp46 polypeptide (e.g., SEQ ID NO:1) is substituted. In the shorthand notation used here, the format is: Wild type residue: Position in polypeptide: Mutant residue, with the numbering of the residues as indicated in SEQ ID NO: 1.

In some embodiments, an anti-NKp46 antibody or protein binds a wild-type NKp46 polypeptide but has decreased binding to a mutant NKp46 polypeptide having a mutation (e.g., an alanine substitution) any one or more of the residues R101, V102, E104 and/or L105 (with reference to SEQ ID NO:1) compared to binding to the wild-type NKp46).

In some embodiments, an anti-NKp46 antibody or protein binds a wild-type NKp46 polypeptide but has decreased binding to a mutant NKp46 polypeptide having a mutation (e.g., an alanine substitution) any one or more of the residues K41, E42, E119, Y121 and/or Y194 (with reference to SEQ ID NO:1) compared to binding to the wild-type NKp46).

In some embodiments, an anti-NKp46 antibody or protein binds a wild-type NKp46 polypeptide but has decreased binding to a mutant NKp46 polypeptide having a mutation (e.g., an alanine substitution) any one or more of the residues P132, E133, 1135, and/or S136 (with reference to SEQ ID NO:1) compared to binding to the wild-type NKp46).

The amino acid sequence of the heavy and light chain variable region of parental antibodies NKp46-1, NKp46-2, NKp46-3 and NKp46-4 are listed herein in Table B. Amino acid sequence of the heavy and light chain variable region of humanized antibodies NKp46-1, NKp46-2, NKp46-3, NKp46-4 and NKp46-9 that displayed high binding affinity for both human and non-human primate NKp46 are listed herein in Table C.

A heavy chain variable region of a NKp46-1, NKp46-2, NKp46-3, NKp-46-4, NKp46-6 or NKp46-9 antibody may comprise, for the respective antibody: a human heavy chain FR1 framework region; a HCDR1 region comprising an amino acid sequence as set forth in Table A, or a sequence of at least 4, 5, 6, 7, 8, 9 or 10 contiguous amino acids thereof, wherein one or more of these amino acids may be substituted by a different amino acid; a human heavy chain FR2 framework region; a HCDR2 region comprising an amino acid sequence as set forth in Table A, or a sequence of at least 4, 5, 6, 7, 8, 9 or 10 contiguous amino acids thereof, wherein one or more of these amino acids may be substituted by a different amino acid; a human heavy chain FR3 framework region; and a HCDR3 region comprising an amino acid sequence as set forth in as set forth in Table A, or a sequence of at least 4, 5, 6, 7, 8, 9 or 10 contiguous amino acids thereof, wherein one or more of these amino acids may be substituted by a different amino acid. Optionally, the variable region further comprises a human heavy chain FR4 framework region.

A light chain variable region of a NKp46-1, NKp46-2, NKp46-3, NKp-46-4, NKp46-6 or NKp46-9 antibody may comprise, for the respective antibody: a human light chain FR1 framework region; a LCDR1 region comprising an amino acid sequence as set forth in Table A, or a sequence of at least 4, 5, 6, 7, 8, 9 or 10 contiguous amino acids thereof, wherein one or more of these amino acids may be substituted by a different amino acid; a human light chain FR2 framework region; a LCDR2 region comprising an amino acid sequence as set forth in Table A, or a sequence of at least 4, 5, 6, 7, 8, 9 or 10 contiguous amino acids thereof, wherein one or more of these amino acids may be substituted by a different amino acid; a human light chain FR3 framework region; and a LCDR3 region comprising an amino acid sequence as set forth in Table A, or a sequence of at least 4, 5, 6, 7, 8, 9 or 10 contiguous amino acids thereof, wherein one or more of these amino acids may be deleted or substituted by a different amino acid. Optionally, the variable region further comprises a human light chain FR4 framework region.

Examples of VH and VL combinations include:

(a) a VH comprising a CDR1, 2 and 3 of SEQ ID NO: 3 and a FR1, 2 and 3 of a human IGHV1-69 gene segment, and a VL comprising a CDR1, 2 and 3 of SEQ ID NO: 4 and a FR1, 2 and 3 of a human IGKV1-33 gene segment;

(b) a VH comprising a CDR1, 2 and 3 of SEQ ID NO: 5 and a FR1, 2 and 3 of a human IGHV4-30-4 gene segment, and a VL comprising a CDR1, 2 and 3 of SEQ ID NO: 6 and a FR1, 2 and 3 of a human IGKV1-39 gene segment;

(c) a VH comprising a CDR1, 2 and 3 of SEQ ID NO: 7 and a FR1, 2 and 3 of a human IGHV1-69 gene segment, and a VL comprising a CDR1, 2 and 3 of SEQ ID NO: 8 and a FR1, 2 and 3 of a human IGKV3-11 and/or IGKV3-15 gene segment;

(d) a VH comprising a CDR1, 2 and 3 of SEQ ID NO: 9 and a FR1, 2 and 3 of a human IGHV1-46 and/or a IGHV1-69 gene segment, and a VL comprising a CDR1, 2 and 3 of SEQ ID NO: 10 and a FR1, 2 and 3 of a human IGKV1-NL1 gene segment; or

(e) a VH comprising a CDR1, 2 and 3 of SEQ ID NO: 13 and a FR1, 2 and 3 of a human IGHV4-30-4 gene segment, and a VL comprising a CDR1, 2 and 3 of SEQ ID NO: 14 and a FR1, 2 and 3 of a human IGKV1-39 gene segment.

In another aspect, examples of VH and VL combinations include:

(a) a VH comprising an amino acid sequence at least 70%, 80%, 90%, 95% or 98% identical to the amino acid sequence of the NKp46-1 H1 or H3 variable domain, and a VL comprising an amino acid sequence at least 70%, 80%, 90%, 95% or 98% identical to the amino acid sequence of the NKp46-1 L1 variable domain;

(b) a VH comprising an amino acid sequence at least 70%, 80%, 90%, 95% or 98% identical to the amino acid sequence of the NKp46-2 H1, H2 or H3 variable domain, and a VL comprising an amino acid sequence at least 70%, 80%, 90%, 95% or 98% identical to the amino acid sequence of the NKp46-2 L1 variable domain;

(c) a VH comprising an amino acid sequence at least 70%, 80%, 90%, 95% or 98% identical to the amino acid sequence of the NKp46-3 H1, H3 or H4 variable domain, and a VL comprising an amino acid sequence at least 70%, 80%, 90%, 95% or 98% identical to the amino acid sequence of the NKp46-3 L1 variable domain;

(d) a VH comprising an amino acid sequence at least 70%, 80%, 90%, 95% or 98% identical to the amino acid sequence of the NKp46-4 H1 variable domain, and a VL comprising an amino acid sequence at least 70%, 80%, 90%, 95% or 98% identical to the amino acid sequence of the NKp46-4 L2 variable domain;

(e) a VH comprising an amino acid sequence at least 70%, 80%, 90%, 95% or 98% identical to the amino acid sequence of the NKp46-9 H2 variable domain, and a VL comprising an amino acid sequence at least 70%, 80%, 90%, 95% or 98% identical to the amino acid sequence of the NKp46-9 L1 or L2 variable domain; or

(f) a VH comprising an amino acid sequence at least 70%, 80%, 90%, 95% or 98% identical to the amino acid sequence of the NKp46-9 H3 variable domain, and a VL comprising an amino acid sequence at least 70%, 80%, 90%, 95% or 98% identical to the amino acid sequence of the NKp46-9 L1 or L2 variable domain.

In one embodiment, the aforementioned CDRs are according to Kabat, e.g. as shown in Table A. In one embodiment, the aforementioned CDRs are according to Chotia numbering, e.g. as shown in Table A. In one embodiment, the aforementioned CDRs are according to IMGT numbering, e.g. as shown in Table A.

In one embodiment, the particular VH or VL comprises an amino acid substitution in a human framework (e.g. a back-mutation) at a Kabat position shown in Example 1; optionally the amino acid residue substituted in Example 1 at the corresponding Kabat position can be specified as being present at the particular position.

In another aspect of any of the embodiments herein, any of the CDRs 1, 2 and 3 of the heavy and light chains may be characterized by a sequence of at least 4, 5, 6, 7, 8, 9 or 10 contiguous amino acids thereof, and/or as having an amino acid sequence that shares at least 50%, 60%, 70%, 80%, 85%, 90% or 95% sequence identity with the particular CDR or set of CDRs listed in the corresponding SEQ ID NO or Table A.

The sequences of the CDRs, according to IMGT, Kabat and Chothia definitions systems, have been summarized in Table A below. The sequences of the variable chains of the antibodies according to the invention are listed in Table B below. In any embodiment herein, a VL or VH sequence can be specified or numbered so as to contain or lack a signal peptide or any part thereof.

TABLE A

| | | HCDR1 | | HCDR2 | | HCDR3 | |
|---|---|---|---|---|---|---|---|
| | CDR | SEQ | | SEQ | | SEQ | |
| mAb | definition | ID | Sequence | ID | Sequence | ID | Sequence |
| NKp46-1 | Kabat | 15 | DYVIN | 18 | EIYPGSGTNYYNEKFKA | 21 | RGRYGLYAMDY |
| | Chotia | 16 | GYTFTDY | 19 | PGSG | 22 | GRYGLYAMD |
| | IMGT | 17 | GYTFTDYV | 20 | GYTFTDYVIYPGSGTN | 23 | ARRGRYGLYAMDY |
| NKp46-2 | Kabat | 31 | SDYAWN | 34 | YITYSGSTSYNPSLES | 36 | GGYYGSSWGVFAY |
| | Chotia | 32 | GYSITSDY | | YSG | 37 | GYYGSSWGVFA |
| | IMGT | 33 | GYSITSDYA | 35 | ITYSGST | 38 | ARGGYYGSSWGVFAY |
| NKp46-3 | Kabat | 46 | EYTMH | 49 | GISPNIGGTSYNQKFKG | 51 | RGGSFDY |
| | Chotia | 47 | GYTFTEY | | PNIG | 52 | GGSFD |
| | IMGT | 48 | GYTFTEYT | 50 | ISPNIGGT | 53 | ARRGGSFDY |
| NKp46-4 | Kabat | 60 | SFTMH | 63 | YINPSSGYTEYNQKFKD | 65 | GSSRGFDY |
| | Chotia | 61 | GYTFTSF | | PSSG | 66 | SSRGFD |
| | IMGT | 62 | GYTFTSFT | 64 | INPSSGYT | 67 | VRGSSRGFDY |
| NKp46-6 | Kabat | 73 | SSWMH | 76 | HIHPNSGISNYNEKFKG | 78 | GGRFDD |
| | Chotia | 74 | GYTFTSS | | PNSG | | GRFD |
| | IMGT | 75 | GYTFTSSW | 77 | IHPNSGIS | 79 | ARGGRFDD |
| NKp46-9 | Kabat | 85 | SDYAWN | 88 | YITYSGSTNYNPSLKS | 89 | CWDYALYAMDC |
| | Chotia | 86 | GYSITSDY | | YSG | 90 | WDYALYAMD |
| | IMGT | 87 | GYSITSDYA | 35 | ITYSGST | 91 | ARCWDYALYAMDC |
| Bab281 | Kabat | 97 | NYGMN | 100 | WINTNTGEPTYAEEFKG | 102 | DYLYYFDY |
| | Chotia | 98 | GYTFTNY | | TNTG | 103 | YLYYFD |
| | IMGT | 99 | GYTFTNYG | 101 | INTNTGEP | 104 | ARDYLYYFDY |
| | | LCDR1 | | LCDR2 | | LCDR3 | |
| | CDR | SEQ | | SEQ | | SEQ | |
| mAb | definition | ID | Sequence | ID | Sequence | ID | Sequence |
| NKp46-1 | Kabat | 24 | RASQDISNYLN | 27 | YTSRLHS | 28 | QQGNTRPWT |
| | Chotia | 25 | SQDISNY | | YTS | 29 | YTSGNTRPW |
| | IMGT | 26 | QDISNY | | YTS | 30 | YTSQQGNTRPWT |
| NKp46-2 | Kabat | 39 | RVSENIYSYLA | 42 | NAKTLAE | 43 | QHHYGTPWT |
| | Chotia | 40 | SENIYSY | | NAK | 44 | HYGTPW |
| | IMGT | 41 | ENIYSY | | NAK | 45 | QHHYGTPWT |
| NKp46-3 | Kabat | 54 | RASQSISDYLH | 57 | YASQSIS | 58 | QNGHSFPLT |
| | Chotia | 55 | SQSISDY | | YAS | 59 | GHSFPL |
| | IMGT | 56 | QSISDY | | YAS | | QNGHSFPLT |
| NKp46-4 | Kabat | 68 | RASENIYSNLA | 70 | AATNLAD | 71 | QHFWGTPRT |
| | Chotia | | SENIYSN | | AAT | 72 | FWGTPR |
| | IMGT | 69 | ENIYSN | | AAT | | QHFWGTPRT |
| NKp46-6 | Kabat | 80 | RASQSISDYLH | | YASQSIS | 82 | QNGHSFLMYT |
| | Chotia | 81 | GRFDSQSISDY | | YAS | 83 | GHSFLMY |
| | IMGT | | QSISDY | | YAS | 84 | YASQNGHSFLMYT |
| NKp46-9 | Kabat | 92 | RTSENIYSYLA | 93 | NAKTLAE | 94 | QHHYDTPLT |
| | Chotia | | SENIYSY | | NAK | 95 | NAKHYDTPL |
| | IMGT | | ENIYSY | | NAK | 96 | QHHYDTPLT |
| Bab281 | Kabat | 105 | KASENVVTYVS | 108 | GASNRYT | 109 | GQGYSYPYT |
| | Chotia | 106 | SENVVTY | | GAS | 110 | GYSYPY |
| | IMGT | 107 | ENVVTY | | GAS | 111 | GQGYSYPYT |

TABLE B

| | SEQ ID | |
|---|---|---|
| Antibody | NO | Amino acid sequence |
| NKp46-1 VH | 3 | QVQLQQSGPELVKPGASVKMSCKASGYTFTDYVINWGKQRSGQGLEWIGEI |
| | | YPGSGTNYYNEKFKAKATLTADKSSNIAYMQLSSLTSEDSAVYFCARRGRY |
| | | GLYAMDYWGQGTSVTVSS |
| NKp46-1 VL | 4 | DIQMTQTTSSLSASLGDRVTISCRASQDISNYLNWYQQKPDGTVKLLIYYT |
| | | SRLHSGVPSRFSGSGSGTDYSLTINNLEQEDIATYFCQQGNTRPWTFGGGT |
| | | KLEIK |
| NKp46-2 VH | 5 | EVQLQESGPGLVKPSQSLSLTCTVTGYSITSDYAWNWIRQFPGNKLEWMGY |
| | | ITYSGSTSYNPSLESRISITRDTSTNQFFLQLNSVTTEDTATYYCARGGYY |
| | | GSSWGVFAYWGQGTLVTVSA |
| NKp46-2 VL | 6 | DIQMTQSPASLSASVGETVTITCRVSENIYSYLAWYQQKQGKSPQLLVYNA |
| | | KTLAEGVPSRESGSGSGTQFSLKINSLQPEDFGSYYCQHHYGTPWTFGGGT |
| | | KLEIK |
| NKp46-3 VH | 7 | EVQLQQSGPELVKPGASVKISCKTSGYTFTEYTMHWVKQSHGKSLEWIGGI |
| | | SPNIGGTSYNQKFKGKATLTVDKSSSTAYMELRSLTSEDSAVYYCARRGGS |
| | | FDYWGQGTTLTVSS |
| NKp46-3 VL | 8 | DIVMTQSPATLSVTPGDRVSLSCRASQSISDYLHWYQQKSHESPRLLIKYA |
| | | SQSISGIPSRFSGSGSGSDFTLSINSVEPEDVGVYYCONGHSFPLTFGAGT |
| | | KLELK |
| NKp46-4 VH | 9 | QVQLQQSAVELARPGASVKMSCKASGYTFTSFTMHWVKQRPGQGLEWIGYI |
| | | NPSSGYTEYNQKFKDKTTLTADKSSSTAYMQLDSLTSDDSAVYYCVRGSSR |
| | | GFDYWGQGTLVTVSA |
| NKp46-4 VL | 10 | DIQMIQSPASLSVSVGETVTITCRASENIYSNLAWFQQKQGKSPQLLVYAA |
| | | TNLADGVPSRFSGSGSGTQYSLKINSLQSEDFGIYYCQHEWGTPRTFGGGT |
| | | KLEIK |
| NKp46-6 VH | 11 | QVQLQQPGSVLVRPGASVKLSCKASGYTFTSSWMHWAKQRPGQGLEWIGHI |
| | | HPNSGISNYNEKFKGKATLTVDTSSSTAYVDLSSLTSEDSAVYYCARGGRF |
| | | DDWGAGTTVTVSS |
| NKp46-6 VL | 12 | DIVMTQSPATLSVTPGDRVSLSCRASQSISDYLHWYQQKSHESPRLLIKYA |
| | | SQSISGIPSRESGSGSGSDFTLSINSVEPEDVGVYYCQNGHSFLMYTFGGG |
| | | TKLEIK |
| NKp46-9 VH | 13 | DVQLQESGPGLVKPSQSLSLTCTVTGYSITSDYAWNWIRQFPGNKLEWMGY |
| | | ITYSGSTNYNPSLKSRISITRDTSKNQFFLQLNSVTTEDTATYYCARCWDY |
| | | ALYAMDCWGQGTSVTVSS |
| NKp46-9 VL | 14 | DIQMTQSPASLSASVGETVTITCRTSENIYSYLAWCQQKQGKSPQLLVYNA |
| | | KTLAEGVPSRESGSGSGTHFSLKINSLQPEDFGIYYCQHHYDTPLTFGAGT |
| | | KLELK |

TABLE C

| | VH | VL |
|---|---|---|
| Antibody | (SEQ ID NO) | (SEQ ID NO) |
| NKp46-1 H1L1 | 199 | 201 |
| NKp46-1 H3L1 | 200 | 201 |
| NKp46-2 H1L1 | 202 | 205 |
| NKp46-2 H2L1 | 203 | 205 |
| NKp46-2 H3L1 | 204 | 205 |
| NKp46-3 H1L1 | 206 | 209 |
| NKp46-3 H3L1 | 207 | 209 |
| NKp46-3 H4L1 | 208 | 209 |
| NKp46-4 H1L2 | 210 | 213 |
| NKp46-4 H2L2 | 211 | 213 |
| NKp46-4 H3L2 | 212 | 213 |
| NKp46-9 H2L1 | 215 | 217 |
| NKp46-9 H2L2 | 215 | 218 |
| NKp46-9 H3L1 | 216 | 217 |
| NKp46-9 H3L2 | 216 | 218 |

### Multi-Specific Antibodies and Polypeptides

Antigen binding domains (ABDs) that bind NKp46 can be derived from the anti-NKp46 CDR, VH and VL sequences provided herein (see section “Anti-NKp46 variable domains”). The antigen binding domains can be arranged one the same or on separate polypeptides, such that they form an antigen binding domain capable of binding NKp46 (e.g. human NKp46 as expressed at the surface of a cell). The ABDs can be produced, for example, as an scFv, a tandem scFv, a Bite, a DART, a Fab, a F(ab)₂, an antibody, a bispecific antibody, or a monomeric or multimeric Fc protein.

For the construction of multi-specific proteins (e.g. antibodies, tandem scFv, BiTe, DART, Fc proteins), antigen binding domains that bind an antigen of interest (other than NKp46) can be readily derived a variety of immunoglobulin or non-immunoglobulin scaffolds, for example affibodies based on the Z-domain of staphylococcal protein A, engineered Kunitz domains, monobodies or adnectins based on the 10th extracellular domain of human fibronectin III, anticalins derived from lipocalins, DARPins (designed ankyrin repeat domains, multimerized LDLR-A module, avimers or cysteine-rich knottin peptides. See, e.g., Gebauer and Skerra (2009) Current Opinion in Chemical Biology 13:245-255, the disclosure of which is incorporated herein by reference.

Variable domains that bind an antigen of interest (other than NKp46) can be derived from an antibody, for example in the form of associated VL and VH domains found on two polypeptide chains, or single chain antigen binding domains such as scFv, a VH domain, a VL domain, a dAb, a V-NAR domain or a VHH domain. The an antigen binding domain (e.g,. ABD1 and ABD2) can also be readily derived from antibodies as a Fab.

Typically, antibodies are initially obtained by immunization of a non-human animal, e.g., a mouse, with an immunogen comprising a polypeptide, or a fragment or derivative thereof, typically an immunogenic fragment, for which it is desired to obtain antibodies (e.g. a human polypeptide). The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, NY (1988), the entire disclosure of which is herein incorporated by reference).

Antigen binding domains (ABDs) that bind antigens of interest (other than NKp46) for use in a multispecific polypeptide can be selected based on the desired cellular target, and may include for example cancer antigens, bacterial or viral antigens, etc. As used herein, the term “bacterial antigen” includes, but is not limited to, intact, attenuated or killed bacteria, any structural or functional bacterial protein or carbohydrate, or any peptide portion of a bacterial protein of sufficient length (typically about 8 amino acids or longer) to be antigenic. Examples include gram-positive bacterial antigens and gram-negative bacterial antigens. In some embodiments the bacterial antigen is derived from a bacterium selected from the group consisting of Helicobacter species, in particular Helicobacter pyloris; Borelia species, in particular Borelia burgdorferi; Legionella species, in particular Legionella pneumophilia; Mycobacteria s species, in particular M. tuberculosis, M. avium, M. intracellulare, M. kansasii, M. gordonae; Staphylococcus species, in particular Staphylococcus aureus; Neisseria species, in particular N. gonorrhoeae, N. meningitidis; Listeria species, in particular Listeria monocytogenes; Streptococcus species, in particular S. pyogenes, S. agalactiae; S. faecalis; S. bovis, S. pneumonias; anaerobic Streptococcus species; pathogenic Campylobacter species; Enterococcus species; Haemophilus species, in particular Haemophilus influenzue; Bacillus species, in particular Bacillus anthracis; Corynebacterium species, in particular Corynebacterium diphtheriae; Erysipelothrix species, in particular Erysipelothrix rhusiopathiae; Clostridium species, in particular C. perfringens, C. tetani; Enterobacter species, in particular Enterobacter aerogenes, Klebsiella species, in particular Klebsiella 1S pneumoniae, Pasturella species, in particular Pasturella multocida, Bacteroides species; Fusobacterium species, in particular Fusobacterium nucleatum; Streptobacillus species, in particular Streptobacillus moniliformis; Treponema species, in particular Treponema pertenue; Leptospira; pathogenic Escherichia species; and Actinomyces species, in particular Actinomyces israelli.

As used herein, the term “viral antigen” includes, but is not limited to, intact, attenuated or killed whole virus, any structural or functional viral protein, or any peptide portion of a viral protein of sufficient length (typically about 8 amino acids or longer) to be antigenic. Sources of a viral antigen include, but are not limited to viruses from the families: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunya viruses, phleboviruses and Nairo viruses); Arenaviridae (hemorrhagic fever viruses); Reoviridae (e.g., reoviruses, orbiviruses and rotaviruses); Bornaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g., African swine fever virus); and unclassified viruses (e.g., the agent of delta hepatitis (thought to be a defective satellite of hepatitis B virus), Hepatitis C; Norwalk and related viruses, and astroviruses). Alternatively, a viral antigen may be produced recombinantly.

As used herein, the terms “cancer antigen” and “tumor antigen” are used interchangeably and refer to antigens (other than NKp46) that are differentially expressed by cancer cells or are expressed by non-tumoral cells (e.g. immune cells) having a pro-tumoral effect (e.g. an immunosuppressive effect), and can thereby be exploited in order to target cancer cells. Cancer antigens are antigens which can potentially stimulate apparently tumor-specific immune responses. Some of these antigens are encoded, although not necessarily expressed, or expressed at lower levels or less frequently, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. Still other cancer antigens can be expressed on immune cells capable of mediating a pro-tumoral effect, e.g. a monocyte or a macrophage, optionally a suppressor T cell, regulatory T cell, or myeloid-derived suppressor cell.

The cancer antigens are usually normal cell surface antigens which are either over-expressed or expressed at abnormal times, or are expressed by a targeted population of cells. Ideally the target antigen is expressed only on proliferative cells (e.g., tumor cells) or pro-tumoral cells (e.g. immune cells having an immunosuppressive effect), however this is rarely observed in practice. As a result, target antigens are in many cases selected on the basis of differential expression between proliferative/disease tissue and healthy tissue. Example of cancer antigens include: Receptor Tyrosine Kinase-like Orphan Receptor 1 (ROR1), Cripto, CD4, CD20, CD30, CD19, CD38, CD47, Glycoprotein NMB, CanAg, Her2 (ErbB2/Neu), a Siglec family member, for example CD22 (Siglec2) or CD33 (Siglec3), CD79, CD138, CD171, PSCA, L1-CAM, PSMA (prostate specific membrane antigen), BCMA, CD52, CD56, CD80, CD70, E-selectin, EphB2, Melanotransferin, Mud 6 and TMEFF2. Examples of cancer antigens also include Immunoglobulin immunoglobulin superfamily (IgSF) such as cytokine receptors, Killer-Ig Like Receptor, CD28 family proteins, for example, Killer-Ig Like Receptor 3DL2 (KIR3DL2), B7-H3, B7-H4, B7-H6, PD-L1, IL-6 receptor. Examples also include MAGE, MART-1/Melan-A, gp100, major histocompatibility complex class I-related chain A and B polypeptides (MICA and MICB), adenosine deaminase-binding protein (ADAbp), cyclophilin b, colorectal associated antigen (CRC)-C017-1A/GA733, protein tyrosine kinase 7(PTK7), receptor protein tyrosine kinase 3 (TYRO-3), nectins (e.g. nectin-4), major histocompatibility complex class I-related chain A and B polypeptides (MICA and MICB), proteins of the UL16-binding protein (ULBP) family, proteins of the retinoic acid early transcript-1 (RAET1) family, carcinoembryonic antigen (CEA) and its immunogenic epitopes CAP-1 and CAP-2, etv6, aml1, prostate specific antigen (PSA), T-cell receptor/CD3-zeta chain, MAGE-family of tumor antigens, GAGE-family of tumor antigens, anti-Mullerian hormone Type II receptor, delta-like ligand 4 (DLL4), DR5, ROR1 (also known as Receptor Tyrosine Kinase-Like Orphan Receptor 1 or NTRKR1 (EC 2.7.10.1), BAGE, RAGE, LAGE-1, NAG, GnT-V, MUM-1, CDK4, MUC family, VEGF, VEGF receptors, Angiopoietin-2, PDGF, TGF-alpha, EGF, EGF receptor, members of the human EGF-like receptor family, e.g., HER-2/neu, HER-3, HER-4 or a heterodimeric receptor comprised of at least one HER subunit, gastrin releasing peptide receptor antigen, Muc-1, CA125, integrin receptors, avß3 integrins, a5Æ1 integrins, allß3-integrins, PDGF beta receptor, SVE-cadherin, IL-8 receptor, hCG, IL-6 receptor, CSF1R (tumor-associated monocytes and macrophages), α-fetoprotein, E-cadherin, α-catenin, Æ-catenin and γ-catenin, p120ctn, PRAME, NY-ESO-1, cdc27, adenomatous polyposis coli protein (APC), fodrin, Connexin 37, Ig-idiotype, p15, gp75, GM2 and GD2 gangliosides, viral products such as human papillomavirus proteins, imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2, although this is not intended to be exhaustive. In one aspect, the antigen of interest is an antigen (e.g. any one of the antigens listed above) capable of undergoing intracellular internalization, for example when bound by an conventional human IgG1 antibody, either in the presence of absence of Fcγ receptor cells. In one aspect, the antigen of interest is a CD19 polypeptide; in one aspect, the multispecific protein comprises an scFv that binds CD19 comprising an amino acid sequence which is at least 60%, 70%, 80%, 85%, 90% or 95% identical to the sequence of the anti-CD19 scFv of the Examples herein, or that comprises the heavy and light chain CDR1, -2 and -3 of the anti-CD19 heavy and light chain variable regions shown herein.

In one embodiment, the ABD that binds an antigen of interest is derived from (e.g. comprises the hypervariable region of, or comprises the CDRs of) a parental antibody that binds an antigen of interest (e.g. a murine antibody, a human antibody) which, when bound to its antigenic target (the antigen of interest on cells), increases or induces down-modulation or intracellular internalization of the antigen of interest. In one embodiment, the antigen of interest is a cancer antigen, e.g. one of the cancer antigens listed above known to internalize (e.g. Immunoglobulin immunoglobulin superfamily (IgSF) members, for example cytokine receptor alpha or beta chains, Killer-Ig Like Receptors, CD28 family proteins, B7-H3, B7-H4, B7-H6, KIR3DL2, PTK7, ROR1, L1-CAM, Siglec family members, EGF receptor and EGF-like receptor family members, EGFR, HER-2, integrins, anti-Mullerian hormone Type II receptor, CSF-1R, and others). In one embodiment, the antigen target is a polypeptide present on an immune cell capable of mediating a pro-tumoral effect, e.g. a monocyte or a macrophage, optionally a suppressor T cell, regulatory T cell, or myeloid-derived suppressor cell.

In one embodiment, the non-NKp46 ABD binds to a cancer antigen, a viral antigen, a microbial antigen, or an antigen present on an infected cell (e.g. virally infected) or on a pro-inflammatory immune cell.

Once appropriate antigen binding domains having desired specificity and/or activity are identified, DNA encoding each of the or ABD can be separately placed, in suitable arrangements, in an appropriate expression vector, together with DNA encoding any elements such as an enzymatic recognition tag, CH1, Cκ, CH2 and/or CH3 domains and any other optional elements (e.g. DNA encoding a hinge region) for transfection into an appropriate host. ABDs will be arranged in an expression vector, or in separate vectors as a function of which type of polypeptide is to be produced, so as to produce the Fc-polypeptides having the desired domains operably linked to one another. The host is then used for the recombinant production of the multispecific polypeptide.

For example, a polypeptide fusion product can be produced from a vector in which the first of the two ABD is operably linked (e.g. directly, via a heavy or light chain CH1, CK or Cλ constant region and/or hinge region) to the N-terminus of a CH2 domain, and the CH2 domain is operably linked at its C-terminus to the N-terminus a CH3 domain. The second of the two ABD can be linked to the polypeptide at either terminus, or can be on a second polypeptide chain that forms a dimer, e.g. heterodimer, with the polypeptide comprising the first ABD. The polypeptide may comprise a full length Fc domain.

The multispecific polypeptide can then be produced in an appropriate host cell or by any suitable synthetic process. A host cell chosen for expression of the multispecific polypeptide is an important contributor to the final composition, including, without limitation, the variation in composition of the oligosaccharide moieties decorating the protein in the immunoglobulin CH2 domain. The host cell may, for example, be of mammalian origin or may be selected from COS-1, COS-7, HEK293, BHK21, CHO, BSC-1, Hep G2, 653, SP2/0, 293, HeLa, myeloma, lymphoma, yeast, insect or plant cells, or any derivative, immortalized or transformed cell thereof.

A range of different protein formats can be prepared using the NKp46-binding VH-VL pairs of the disclosure, including monomeric, heterodimeric, hetertrimeric and tetrameric multispecific proteins. Included, without limitation, are any of a variety of formats described in for example, in PCT application number PCT/EP2016/064537, filed 23 Jun. 2016 (Innate Pharma), the disclosure of which is incorporated herein by reference.

Monomeric Proteins

In one example, a multispecific protein comprises, in a single polypeptide chain, a first antigen binding domain that binds to NKp46 (e.g. an ABD comprising a VH and a VL disclosed herein, separated by a (poly)peptide linker) and a second antigen binding domain that binds an antigen other than NKp46. In one embodiment, the protein or polypeptide is or comprises an scFv comprising the anti-NKp46 VH and VL of the disclosure, or a tandem scFv comprising the scFv that binds NKp46 and a second scFv that binds NKp46 or an antigen of interest other than NKp46, linked by a (poly)peptide linker optionally fused to another polypeptide or amino acid sequence.

Examples of such single chain antigen binding proteins include BiTE and DART protein formats. An scFv-based bispecific antibody construct, known as BITE® (Bispecific T-cell Engager) employs a single polypeptide containing two antigen-binding domains, each contributed by a pair of VH and VL, linked in tandem via a flexible linker (see, e.g., Nagorsen et al., 2009, Leukemia & Lymphoma 50:886-91; Amann et al., 2009, J Immunother 32:453-64; Baeuerle and Reinhardt, 2009, Cancer Res 69:4941-44). Another bispecific antibody called DART® (Dual-Affinity Re-Targeting) utilizes a disulfide-stabilized diabody design (see, e.g., Moore et al., 2011, Blood 117:4542-51; Veri et al., 2010, Arthritis Rheum 62:1933-43).

In one embodiment, the single polypeptide chain further comprises an Fc domain (e.g. a full length Fc domain or a portion thereof), optionally wherein the Fc domain is interposed between the first and second antigen binding domains.

In one aspect of any embodiment, the first antigen binding domain and/or the second antigen binding domain comprise a scFv, optionally where the scFv comprises human framework amino acid sequences. In one embodiment, provided is a monomeric bispecific Fc-derived polypeptide comprising: (a) a first scFv that binds to NKp46; (b) a second scFv that binds an antigen other than NKp46; and, optionally, (c) at least a portion of a human Fc domain. Optionally the Fc domain (i) does not dimerize with another Fc-derived polypeptide and (ii) is capable of binding to human FcRn. Optionally, the Fc domain is interposed between the first and second scFv.

When the polypeptide fusion product comprising the two ABDs and at least a portion of an Fc domain is a monomer, the CH3 domains may be arranged and/or comprise amino acid modification to prevent CH3-CH3 dimerization. In one embodiment, the CH3 domain comprises mutations in the dimer interface to prevent interchain CH3-CH3 dimerization. In another embodiment, the CH3 domain is a tandem CH3 domain (or the Fc domain comprises a tandem CH3 domain) to prevent interchain CH3-CH3 dimerization. Such monomers will retain partial FcRn binding (compared, e.g., to a wild type full length human IgG1 antibody). Optionally the monomeric polypeptide is capable of binding to human FcRn with intermediate affinity, e.g. retains binding to FcRn but has decreased binding to a human FcRn receptor compared to a full-length wild type human IgG1 antibody. The Fc moiety may further comprise one or more amino acid modifications, e.g. in the CH2 domain, that further decreases or substantially abolishes binding to one or more Fcy receptors.

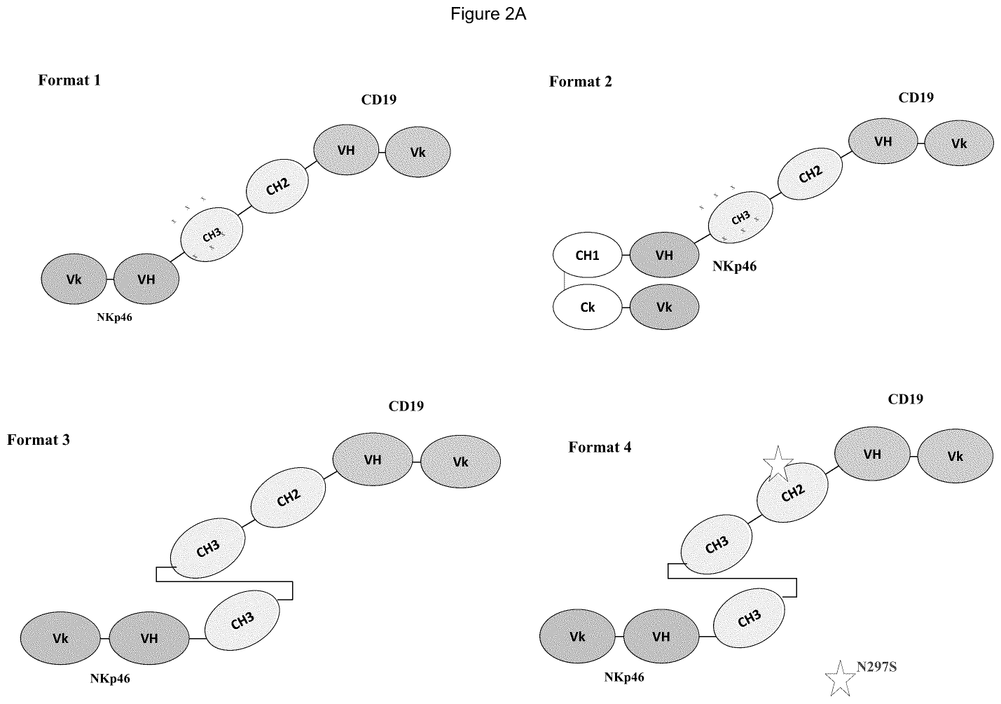

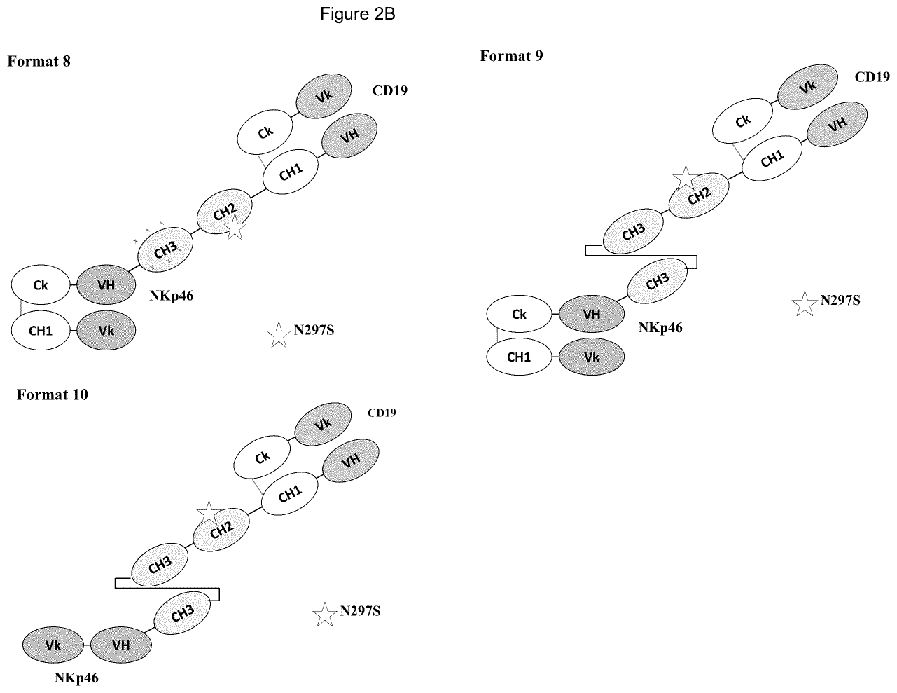

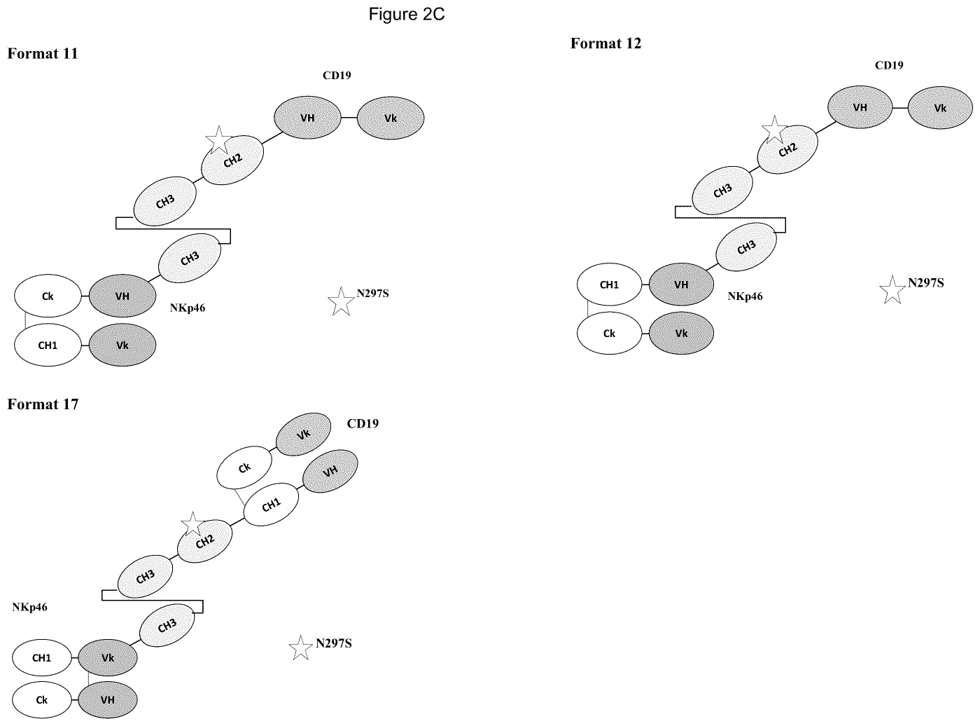

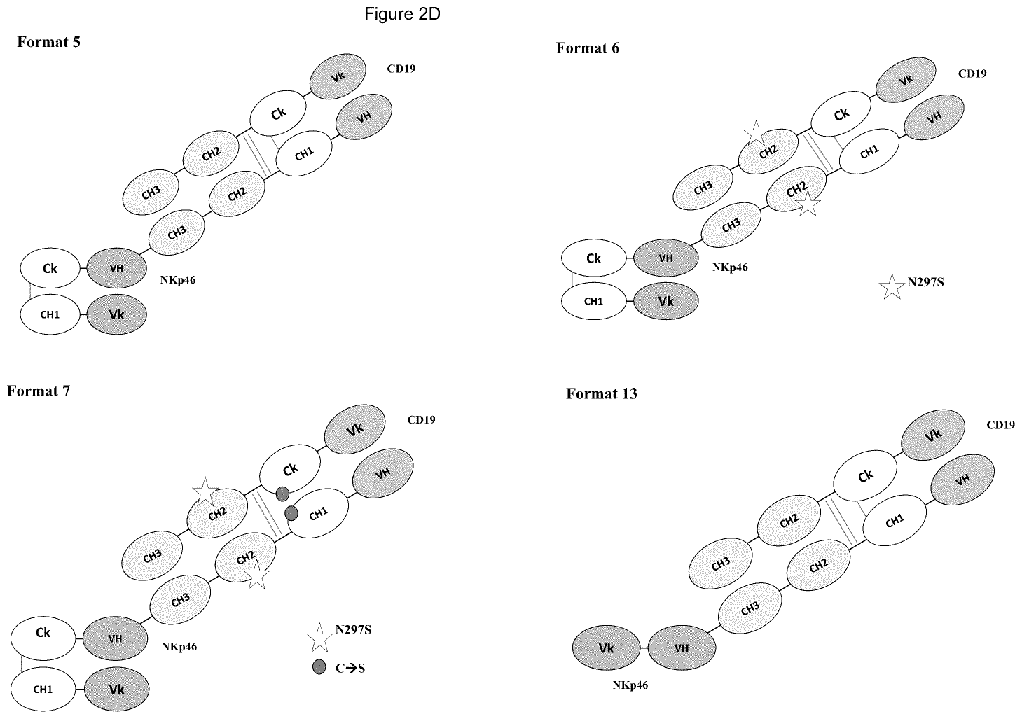

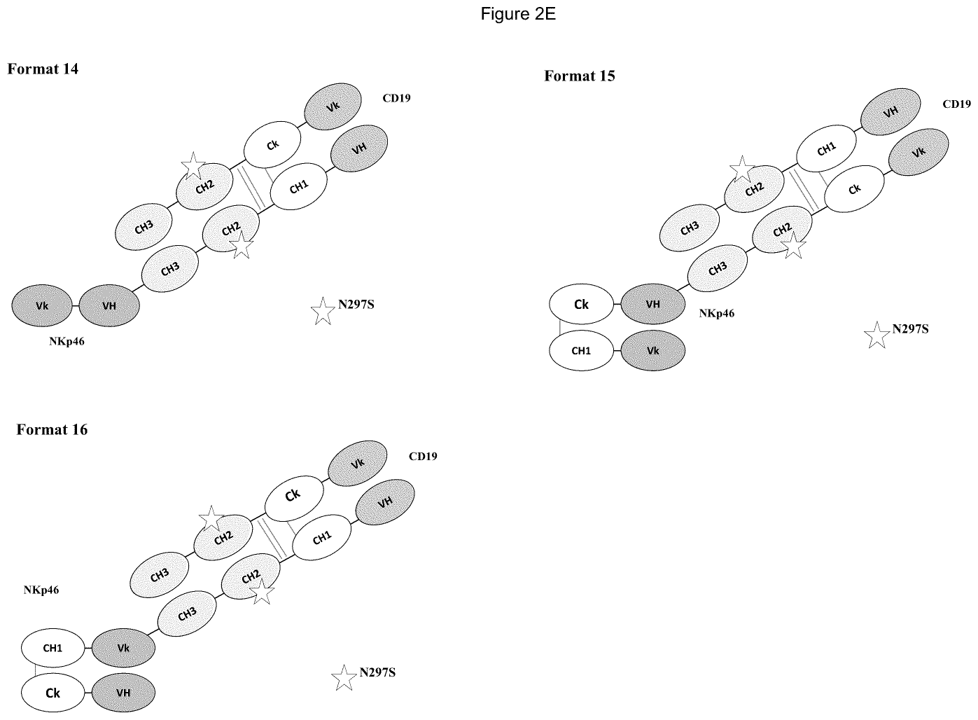

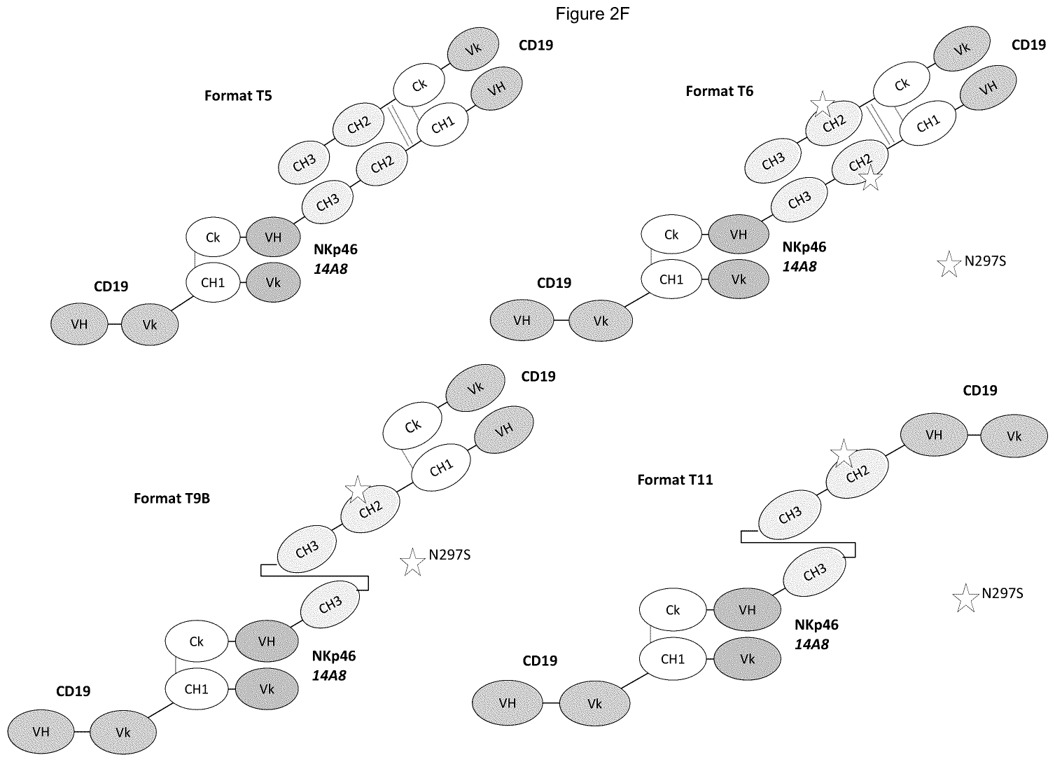

In one configuration, the monomeric Fc-derived polypeptides that have at least a portion of a human Fc domain can advantageously comprise a CH2 domain and a CH3 domain, wherein said CH3 domain comprises a modified CH3 dimer interface (e.g. a mutations in the CH3 dimer interface) to prevent dimerization with another Fc-derived polypeptide. See e.g. format 1 and 2 in FIG. 2A). In one embodiment of any of the polypeptides or methods herein, the CH3 domain comprises an amino acid substitution at 1, 2, 3, 4, 5, 6 or 7 of the positions L351, T366, L368, P395, F405, T407 (or Y407) and/or K409 (EU numbering as in Kabat).

Another configuration for a CH3 domain that can be used in a monomeric multispecific protein is a tandem CH3 domain (see e.g. format 3 and 4 in FIG. 2A). A tandem CH3 domain comprises a first and a second CH3 domain, wherein the two CH3 domains associate with one another via non-covalent interactions. In one embodiment, the two CH3 domains associate with one another via the CH3 dimerization interface of each CH3 domain. In one embodiment, the polypeptide chain does not dimerize with another polypeptide chain comprising an Fc domain. An Fc domain that comprise a tandem CH3 domain will interact with neonatal Fc receptor (FcRn) but will have low or no binding to human Fcγ receptors, notably CD16.

Multimeric Proteins

Multimeric bispecific proteins such as heterodimers, heterotrimers and tetramers (the latter including for example antibodies with two heavy chains and two light chains) that comprise an ABD that binds NKp46 (e.g. an ABD comprising a VH and a VL disclosed herein) can be produced according to a variety of formats.

In one embodiment, a multimeric protein or polypeptide is a tetrameric antibody made up of two heavy chains comprising variable regions (or 1, 2 or 3 CDRs thereof) derived from two different parental antibodies, and two light chains comprising variable regions (or 1, 2 or 3 CDRs thereof) derived from two different parental antibodies. Such a tetramer may comprise (a) two heavy chains each comprising a variable region, a CH1 domain, hinge and an Fc domain, and (b) two antibody light chains each comprising a light chain variable region and a CK domain, wherein one heavy chain variable region together with a light chain variable region binds to NKp46 and the other heavy chain variable region together with a light chain variable region bind an antigen of interest.

One advantageous way of making multimeric proteins is through the assembly of different polypeptide chains that each comprise at least one heavy or light chain variable domain fused to a human CH1 or CK constant domain (a V-(CH1/Cκ) unit), wherein the protein chains undergo CH1-Cκ dimerization and are bound to one another by non-covalent bonds and optionally further by disulfide bonds formed between respective CH1 and Cκ domain. In one embodiment, provided is an isolated or purified heterodimeric or heterotimeric protein that binds a first and second antigen, wherein the protein comprises at least two or three polypeptide chains each comprising a V-(CH1/Cκ) unit, whereby the chains are bound to one another by non-covalent bonds and optionally further by disulfide bonds between CH1 and Cκ domains, optionally, whereby the chains are further bound by non-covalent bonds between respective variable regions and CH3 domains of the Fc portion.

The variable and constant regions can be selected and configured such that each chain will preferentially associate with its desired complementary partner chain. The resulting multimeric protein will therefore be simple to produce using conventional production methods using recombinant host cells. The choice of which VH, VL to associate with a CH1 and Cκ in a unit is based on affinity between the units to be paired so as to drive the formation of the desired multimer. The resulting multimer will be bound by non-covalent bonds between complementary VH and VL domains, by non-covalent bonds between complementary CH1 and Cκ domains, and optionally by disulfide bonding between complementary CH1 and Cκ domains and/or optionally further by disulfide bonds between complementary hinge domains). VH-VL associations are stronger than VH-VH or VL-VL, consequently, as shown herein, one can place a VH or a VL next to either a CH1 or a Cκ, and the resulting V-C unit will partner preferably with its V-C counterpart. For example VH-Cκ will pair with VL-CH1 preferentially over VH-CH1. Additionally, by including an Fc domain, preferred chain pairing is further improved, as the two Fc-containing chains will be bound by non-covalent bonds between CH3 domains of the Fc domains. The different V-C combinations, optionally further combined with Fc pairing thereby provides tools to make heteromultimeric proteins.

In one embodiment, a heteromultimeric polypeptide or protein comprises a monomeric Fc domain (e.g. the second polypeptide does not comprise an Fc domain), optionally wherein the Fc domain comprises a CH3 domain with an amino acid mutation to prevent CH3-CH3 dimerization or a tandem CH3 domain.

In another embodiment, the above heteromultimeric polypeptide or protein comprises a dimeric Fc domain capable of binding to human CD16, e.g. a human Fc domain comprising N-linked glycosylation at amino acid residue N297 (Kabat EU numbering).

A heterodimer can for example have the configuration as follows (see also Examples of such proteins shown as formats 2, 11 and 12 shown in FIGS. 2A and 2C):

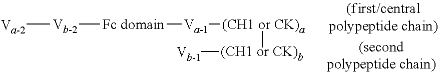

wherein one of Vₐ₋₁ of the first polypeptide chain and V_{b-}1 of the second polypeptide chain is a light chain variable domain and the other is a heavy chain variable domain, and wherein one of Vₐ₋₂ and V_{b-2} is a light chain variable domain and the other is a heavy chain variable domain.

The heterodimer can in another example have the configuration as follows (see also Examples of such proteins shown as format 10 shown in FIG. 2B):

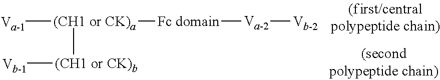

wherein one of Vₐ₋₁ of the first polypeptide chain and V_{b-1} of the second polypeptide chain is a light chain variable domain and the other is a heavy chain variable domain, and wherein one of Vₐ₋₂ and V_{b-2} is a light chain variable domain and the other is a heavy chain variable domain.

The resulting heterodimer can in another example have the configuration as follows (see also Examples of such proteins shown as formats 13 and 14 shown in FIG. 2D and 2E):

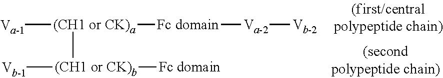

wherein one of Vₐ₋₁ of the first polypeptide chain and V_{b-1} of the second polypeptide chain is a light chain variable domain and the other is a heavy chain variable domain, and wherein one of Vₐ₋₂ and V_{b-2} is a light chain variable domain and the other is a heavy chain variable domain.

Heterotrimeric proteins can for example be formed by using a central (first) polypeptide chain comprising a first variable domain (V) fused to a first CH1 or CK constant region, a second variable domain (V) fused to a second CH1 or CK constant region, and an Fc domain or portion thereof interposed between the first and second variable domains (i.e. the Fc domain is interposed between the first and second (V-(CH1/CK) units. For example, a central polypeptide chain for use in a heterotrimeric protein can have the domain arrangements (N- to C-terminal) as follows:

Vₐ₋₁-(CH1 or CK)ₐ-Fc domain-Vₐ₋₂-(CH1 or CK)_{b}.

A second polypeptide chain can then comprise a domain arrangement (N- to C-terminal):

V_{b-1}-(CH1 or CK)_{c},

or

V_{b-1}-(CH1 or CK)_{c}-Fc domain

such that the (CH1 or CK)_{c} dimerizes with the (CH1 or CK)ₐ on the central chain, and the Vₐ₋₁ and V_{b-1} form an antigen binding domain.

A third polypeptide chain can then comprise a domain arrangement (N- to C-terminal):

V_{b-2}-(CH1 or CK)_{d},

such that the (CH1 or CK)_{d} dimerizes with the (CH1 or CK)_{b} unit on the central chain, and the Vₐ₋₂ and V_{b-2} form an antigen binding domain.

An example of a configuration of a resulting heterotrimer with a dimeric Fc domain (also shown as formats 5, 6, 7 and 16 in FIGS. 2D and 2E) has a domain arrangement:

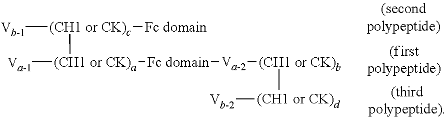

An example of a configuration of a resulting heterotrimer with a monomeric Fc domain (also shown as formats 8, 9 and 17 in FIGS. 2B and 2C) has a domain arrangement:

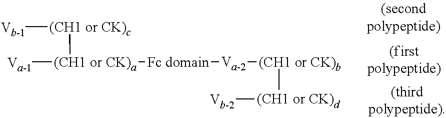

Thus, in a configuration of a trimer polypeptide, the first polypeptide can have two variable domains that each form an antigen binding domain with a variable domain on a separate polypeptide chain (i.e. the variable domain of the second and third chains), the second polypeptide chain has one variable domain, and the third polypeptide has one variable domain.

A Trimeric Polypeptide may Comprise:

(a) a first polypeptide chain comprising a first variable domain (V) fused to a first CH1 of CK constant region, a second variable domain (V) fused to a second CH1 of CK constant region, and an Fc domain or portion thereof interposed between the first and second variable domains;

(b) a second polypeptide chain comprising a variable domain fused at its C-terminus to a CH1 or CK constant region selected to be complementary to the first CH1 or CK constant region of the first polypeptide chain such that the first and second polypeptides form a CH1-CK heterodimer, and optionally an Fc domain; and

(c) a third polypeptide chain comprising a variable domain fused (e.g. at its C-terminus) to a CH1 or CK constant region, wherein the variable domain and the constant region are selected to be complementary to the second variable domain and second CH1 or CK constant region of the first polypeptide chain such that the first and third polypeptides form a CH1-CK heterodimer bound by non-covalent bonds and optionally further by disulfide bond(s) formed between the CH1 or CK constant region of the third polypeptide and the second CH1 or CK constant region of the first polypeptide, but not between the CH1 or CK constant region of the third polypeptide and the first CH1 or CK constant region of the first polypeptide;

wherein the first, second and third polypeptides form a CH1-CK heterotrimer, and wherein the first variable domain of the first polypeptide chain and the variable domain of the second polypeptide chain form an antigen binding domain specific for a first antigen of interest, and the second variable domain of the first polypeptide chain and the variable domain on the third polypeptide chain form an antigen binding domain specific for a second antigen of interest. One of the two antigens of interest will be NKp46, and the ABD that binds NKp46 comprises a VH-VL variable domain pair of the disclosure.

Examples of domain arrangement for the trimeric bispecific polypeptide formed from include but are not limited to:

| | |
|---|---|
| 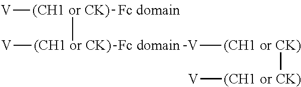 | (second polypeptide) (first polypeptide) (third polypeptide) |
| 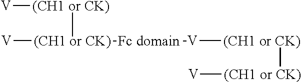 | (second polypeptide) (first polypeptide) (third polypeptide) |
| 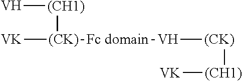 | (second polypeptide) (first polypeptide) (third polypeptide) |
| 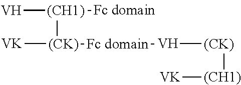 | (second polypeptide) (first polypeptide) (third polypeptide) |
| 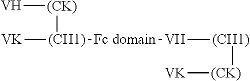 | (second polypeptide) (first polypeptide) (third polypeptide) |
| 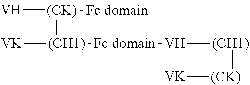 | (second polypeptide) (first polypeptide) (third polypeptide) |

In any of the domain arrangements, the Fc domain may comprise a CH2-CH3 unit (a full length CH2 and CH3 domain or a fragment thereof). In heterodimers or heterotrimers comprising two chains with Fc domains (a dimeric Fc domain), the CH3 domain will be capable of CH3-CH3 dimerization (e.g. a wild-type CH3 domain). In heterodimers or heterotrimers comprising only one chain with an Fc domain (monomeric Fc domain), the Fc domain will be incapable of CH3-CH3 dimerization; for example the CH3 domain(s) will have amino acid modification(s) in the CH3 dimer interface or the Fc domain will comprise a tandem CH3 domain incapable of CH3-CH3 dimerization.

In some exemplary configurations, the multispecific protein can be tetramers, e.g. heterodimeric tetramers with two light chains and two different heavy chains, wherein the heavy chains are engineered for heterodimerization. Such proteins can be prepared as described, for example, in PCT application number PCT/EP2016/064537, filed 23 Jun. 2016 (Innate Pharma), the disclosure of which is incorporated herein by reference.

In any protein of the disclosure, a hinge region will typically be present on a polypeptide chain between a CH1 domain and a CH2 domain, and/or can be present between a CK domain and a CH2 domain. A hinge region can optionally be replaced for example by a suitable linker peptide.

The proteins domains described in the present disclosure can optionally be specified as being from N- to C-terminal. Protein arrangements of the disclosure for purposes of illustration are shown from N-terminus (on the left) to C-terminus. Domains can be referred to as fused to one another (e.g. a domain can be said to be fused to the C-terminus of the domain on its left, and/or a domain can be said to be fused to the N-terminus of the domain on its right).

The proteins domains described in the present disclosure can be fused to one another directly or via intervening amino acid sequences. For example, a CH1 or CK domain will be fused to an Fc domain (or CH2 or CH3 domain thereof) via a linker peptide, optionally a hinge region or a fragment thereof. In another example, a VH or VK domain will be fused to a CH3 domain via a linker peptide. VH and VL domains linked to another in tandem will be fused via a linker peptide (e.g. as an scFv). VH and VL domains linked to an Fc domain will be fused via a linker peptide. Two polypeptide chains will be bound to one another (indicated by “”) by non-covalent bonds and optionally further by interchain disulfide bonds formed between cysteine residues within complementary CH1 and CK domains.

Linkers and Fc domains are described in more detail, for example, in PCT application number PCT/EP2016/064537, filed 23 Jun. 2016 (Innate Pharma), the disclosure of which is incorporated herein by reference.

Once the multispecific protein is produced it can be assessed for biological activity.

In one aspect of any embodiment herein, a multispecific protein is capable of inducing activation of an NKp46-expressing cell (e.g. an NK cell, a reporter cell) when the protein is incubated in the presence of the NKp46-expressing cell (e.g. purified NK cells) and a target cell that expresses the antigen of interest.

In one aspect of any embodiment herein, a multispecific protein is capable of inducing NKp46 signaling in an NKp46-expressing cell (e.g. an NK cell, a reporter cell) when the protein is incubated in the presence of the NKp46-expressing cell (e.g. purified NK cells) and a target cell that expresses the antigen of interest.

Optionally, NK cell activation or signaling in characterized by increased expression of a cell surface marker of activation, e.g. CD107, CD69, etc.

Activity can be measured for example by bringing target cells and NKp46-expressing cells into contact with one another, in presence of the multispecific polypeptide. In one example, aggregation of target cells and NK cells is measured. In another example, the multispecific protein may, for example, be assessed for the ability to cause a measurable increase in any property or activity known in the art as associated with NK cell activity, respectively, such as marker of cytotoxicity (CD107) or cytokine production (for example IFN-γ or TNF-α), increases in intracellular free calcium levels, the ability to lyse target cells in a redirected killing assay, etc. Assays for activity are further described in more detail, for example, in PCT application number PCT/EP2016/064537, filed 23 Jun. 2016 (Innate Pharma), the disclosure of which is incorporated herein by reference.

### Uses of Compounds

Compounds according to the disclosure that comprise an antigen binding domain that binds NKp46 can be used in a variety of applications, including, e.g. to bind, detect, eliminate, purify or modulate the activity of NKp46 polypeptides and/or cells that express NKp46 polypeptide (e.g. NK cells).

In one aspect, provided are the use of any of the compounds defined herein for the manufacture of a pharmaceutical preparation for the treatment or diagnosis of a mammal in need thereof. Provided also are the use any of the compounds defined above as a medicament or an active component or active substance in a medicament. In a further aspect provided is a method for preparing a pharmaceutical composition containing a compound as defined above, to provide a solid or a liquid formulation for administration orally, topically, or by injection. Such a method or process at least comprises the step of mixing the compound with a pharmaceutically acceptable carrier.

In one aspect, provided is a method to treat, prevent or more generally affect a predefined condition by exerting a certain effect, or detect a certain condition using a multispecific protein described herein, or a (pharmaceutical) composition comprising such.

For example, in one aspect, the invention provides a method of restoring or potentiating the activity of NKp46⁺ NK cells in a patient in need thereof (e.g. a patient having a cancer, or a viral or bacterial infection), comprising the step of administering a multispecific protein described herein to said patient. In one embodiment, the method is directed at increasing the activity of NKp46+ lymphocytes in patients having a disease in which increased lymphocyte (e.g. NK cell) activity is beneficial or which is caused or characterized by insufficient NK cell activity, such as a cancer, or a viral or microbial/bacterial infection.

The polypeptides described herein can be used to prevent or treat disorders that can be treated with antibodies, such as cancers, solid and non-solid tumors, hematological malignancies, infections such as viral infections, and inflammatory or autoimmune disorders. In one embodiment, the antigen of interest (the non-NKp46 antigen) is an antigen expressed on the surface of a malignant cell of a type of cancer selected from the group consisting of: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; Sezary syndrome (SS); Adult T-cell leukemia lymphoma (ATLL); a/d T-NHL hepatosplenic lymphoma; peripheral/post-thymic T cell lymphoma (pleomorphic and immunoblastic subtypes); angio immunoblastic T-cell lymphoma; angiocentric (nasal) T-cell lymphoma; anaplastic (Ki 1+) large cell lymphoma; intestinal T-cell lymphoma; T-lymphoblastic; and lymphoma/leukaemia (T-Lbly/T-ALL).

In one embodiment, polypeptides described herein can be used to prevent or treat a cancer selected from the group consisting of: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma. Other exemplary disorders that can be treated according to the invention include hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; Sezary syndrome (SS); Adult T-cell leukemia lymphoma (ATLL); a/d T-NHL hepatosplenic lymphoma; peripheral/post-thymic T cell lymphoma (pleomorphic and immunoblastic subtypes); angio immunoblastic T-cell lymphoma; angiocentric (nasal) T-cell lymphoma; anaplastic (Ki 1+) large cell lymphoma; intestinal T-cell lymphoma; T-lymphoblastic; and lymphoma/leukaemia (T-Lbly/T-ALL).

In one example, the tumor antigen is an antigen expressed on the surface of a lymphoma cell or a leukemia cell, and the multispecific protein is administered to, and/or used for the treatment of, an individual having a lymphoma or a leukemia. Optionally, the tumor antigen is selected from CD19, CD20, CD22, CD30 or CD33.

In one aspect, the methods of treatment comprise administering to an individual a multispecific protein described herein in a therapeutically effective amount, e.g., for the treatment of a disease as disclosed herein, for example a cancer selected from the group above. A therapeutically effective amount may be any amount that has a therapeutic effect in a patient having a disease or disorder (or promotes, enhances, and/or induces such an effect in at least a substantial proportion of patients with the disease or disorder and substantially similar characteristics as the patient).

In one embodiment, the multispecific protein described herein may be used as monotherapy (without other therapeutic agents), or in combined treatments with one or more other therapeutic agents, including agents normally utilized for the particular therapeutic purpose for which the antibody is being administered. The additional therapeutic agent will normally be administered in amounts and treatment regimens typically used for that agent in a monotherapy for the particular disease or condition being treated. Such therapeutic agents when used in the treatment of cancer, include, but are not limited to anti-cancer agents and chemotherapeutic agents; in the treatment of infectious disease, include, but are not limited to anti-viral agents and anti-biotics.

The proteins and/or polypeptides disclosed herein can be included in kits. The kits may optionally further contain any number of polypeptides and/or other compounds, e.g., 1, 2, 3, 4, or any other number of proteins and/or polypeptides and/or other compounds. It will be appreciated that this description of the contents of the kits is not limiting in any way. For example, the kit may contain other types of therapeutic compounds. Optionally, the kits also include instructions for using the proteins and/or polypeptides, e.g., detailing the herein-described methods.

Also provided are pharmaceutical compositions comprising the compounds as defined above. A compound may be administered in purified form together with a pharmaceutical carrier as a pharmaceutical composition. The form depends on the intended mode of administration and therapeutic or diagnostic application. The pharmaceutical carrier can be any compatible, nontoxic substance suitable to deliver the compounds to the patient. Pharmaceutically acceptable carriers are well known in the art and include, for example, aqueous solutions such as (sterile) water or physiologically buffered saline or other solvents or vehicles such as glycols, glycerol, oils such as olive oil or injectable organic esters, alcohol, fats, waxes, and inert solids A pharmaceutically acceptable carrier may further contain physiologically acceptable compounds that act for example to stabilize or to increase the absorption of the compounds Such physiologically acceptable compounds include, for example, carbohydrates, such as glucose, sucrose or dextrans, antioxidants, such as ascorbic acid or glutathione, chelating agents, low molecular weight proteins or other stabilizers or excipients One skilled in the art would know that the choice of a pharmaceutically acceptable carrier, including a physiologically acceptable compound, depends, for example, on the route of administration of the composition Pharmaceutically acceptable adjuvants, buffering agents, dispersing agents, and the like, may also be incorporated into the pharmaceutical compositions.

The compounds can be administered parenterally. Preparations of the compounds for parenteral administration must be sterile. Sterilization is readily accomplished by filtration through sterile filtration membranes, optionally prior to or following lyophilization and reconstitution. The parenteral route for administration of compounds is in accord with known methods, e.g. injection or infusion by intravenous, intraperitoneal, intramuscular, intraarterial, or intralesional routes. The compounds may be administered continuously by infusion or by bolus injection. A typical composition for intravenous infusion could be made up to contain 100 to 500 ml of sterile 0.9% NaCl or 5% glucose optionally supplemented with a 20% albumin solution and 1 mg to 10 g of the compound, depending on the particular type of compound and its required dosing regimen. Methods for preparing parenterally administrable compositions are well known in the art.

### EXAMPLES

### Example 1

### Generation of Anti-huNKp46 Antibodies

Part A: Generation of Anti-huNKp46 Antibodies

Balb/c mice were immunized with a recombinant human NKp46 extracellular domain recombinant-Fc protein. Mice received one primo-immunization with an emulsion of 50 μg NKp46 protein and Complete Freund Adjuvant, intraperitoneally, a 2nd immunization with an emulsion of 50 μg NKp46 protein and Incomplete Freund Adjuvant, intraperitoneally, and finally a boost with 10 μg NKp46 protein, intravenously. Immune spleen cells were fused 3 days after the boost with X63.Ag8.653 immortalized B cells, and cultured in the presence of irradiated spleen cells.

Primary screen: Supernatant (SN) of growing clones were tested in a primary screen by flow cytometry using a cell line expressing the human NKp46 construct at the cell surface. Briefly, for FACS screening, the presence of reacting antibodies in supernanants was revealed by Goat anti-mouse polyclonal antibody (pAb) labeled with PE.

A selection of antibodies that bound NKp46 were selected, produced as full-length human IgG antibodies and as bispecific antibodies. Among the variable regions evaluated for their activity in the context of a bispecific molecule in Examples 2-13 were antibodies NKp46-1, -2, -3, -4, -6 and -9 having the respective variable regions shown in Table B herein.

Part B : Generation of Humanized Anti-Human/Anti-Cynomologus NKp46 Antibodies

Antibodies NKp46-1, -2, -3 and -4 having the respective variable regions shown in Table B herein were produced as humanized antibodies by complementary determining region (CDR) grafting of heavy and light chains having the amino acid sequence shown below. Antibodies were produced using CHO cells and tested for binding to human NKp46.

Each of the CDR-grafted antibodies bound with good affinity to human NKp46. However, none of the CDR-grafted antibodies bound to cynomologus NKp46. Epitopes on human NKp46 were determined (see Example 13); in view of the possibility that modifications that affect the conformation of the antibody variable regions or positioning of the CDRs may permit an epitope shared on cynomolgus NKp46 to be recognized, multiple variants were prepared for each of the CDR-grafted antibodies for NKp46-1, -2, -3, -4, and -9 were prepared and produced using CHO cells, and tested for binding to cynomolgus NKp46. For each of antibody NKp46-1, -2, -3, -4, and -9, one more variants were identified that permitted the recognition with good affinity of an epitope shared by human and cynomolgus NKp46 binding. Based on 3D modelling studies, different heavy and light chain variable regions were designed that included NKp46-1 CDRs and human frameworks, produced as human IgG1 antibodies, and tested for binding to cynomolgus NKp46.

Antibody NKp46-1

Based on 3D modelling studies, different heavy and light chain variable regions were designed that included NKp46-1 CDRs and human frameworks, produced as human IgG1 antibodies, and tested for binding to cynomolgus NKp46. Two combinations of heavy and light chains were able to bind to both human and cynomolgus NKp46: the heavy chain variable region “H1” and the heavy chain “H3”, in each case combined with the light chain “L1”. These cross-binding variable regions included, for the heavy chain variable region: the NKp46-1 heavy chain CDRs (shown below, underlined), human IGHV1-69*06 gene framework 1, 2 and 3 regions and a human IGHJ6*01 gene framework 4 region. The light chain variable region: the NKp46-1 light chain CDRs (shown below, underlined), human IGKV1-33*01 gene framework 1, 2 and 3 regions and a human IGKJ4*01 gene framework 4 region. CDRs were chosen according to Kabat numbering. The H1, H3 and L1 chain had the specific amino acid substitutions (shown in bold and underlining below). L1 had a phenylalanine at Kabat light chain residue 87. H1 had a tyrosine at Kabat heavy chain residue 27 and a lysine and alanine at Kabat residues 66 and 67, respectively. H3 additionally had a glycine at Kabat residue 37, an isoleucine at Kabat residue 48, and a phenylalanine at Kabat residue 91.

NKp46-1: “H1” heavy chain variable region
(SEQ ID NO: 199)
QVQLVQSGAEVKKPGSSVKVSCKASGYTFSDYVINWVRQAPGQGLEWMGE
IYPGSGTNYYNEKFKAKATITADKSTSTAYMELSSLRSEDTAVYYCARRG
RYGLYAMDYWGQGTTVTVSS
NKp46-1: “H3” heavy chain variable region
(SEQ ID NO: 200)
QVQLVQSGAEVKKPGSSVKVSCKASGYTFTDYVINWGRQAPGQGLEWIGE
IYPGSGTNYYNEKFKAKATITADKSTSTAYMELSSLRSEDTAVYFCARRG
RYGLYAMDYWGQGTTVTVSS
NKp46-1: “L1” light chain variable region
(SEQ ID NO: 201)
DIQMTQSPSSLSASVGDRVTITCRASQDISNYLNWYQQKPGKAPKLLIYY
TSRLHSGVPSRFSGSGSGTDFTFTISSLQPEDIATYFCQQGNTRPWTFGG
GTKVEIK

Antibody NKp46-2

Based on 3D modelling studies, different heavy and light chain variable regions were designed that included NKp46-2 CDRs and human frameworks, produced as human IgG1 antibodies, and tested for binding to cynomolgus NKp46. Three combinations of heavy and light chains were able to bind to both human and cynomolgus NKp46: the heavy chain variable regions “H1”, “H2 and “H3”, in each case combined with the light chain “L1”. Interestingly, the H1L1 furthermore had an improved binding affinity compared to the parental NKp46-2 antibody having the VH and VL of SEQ ID NOS: 5 and 6. These cross-binding variable regions included, for the heavy chain variable region: the NKp46-2 heavy chain CDRs (shown below, underlined), human IGHV4-30-4*01 gene framework 1, 2 and 3 regions and a human IGHJ1*01 gene framework 4 region. The light chain variable region: the NKp46-2 light chain CDRs (shown below, underlined), human IGKV1-39*01 gene framework 1, 2 and 3 regions and a human IGKJ4*01 gene framework 4 region. CDRs were chosen according to Kabat numbering. The L1, H1, H2 and H3 chains had the specific amino acid substitutions (shown in bold and underlining below). L1 had a valine at Kabat light chain residue 48. H1 had a tyrosine at Kabat heavy chain residue 27 and an arginine at Kabat residue 71. H2 additionally had a methionine at Kabat residue 48 and an isoleucine at Kabat residue 67. H3 additionally had a threonine at Kabat residue 31.

NKp46-2: “H1” heavy chain variable region
(SEQ ID NO: 202)
QVQLQESGPGLVKPSQTLSLTCTVSGYSISSDYAWNWIRQPPGKGLEWIG
YITYSGSTSYNPSLESRVTISRDTSKNQFSLKLSSVTAADTAVYYCARGG
YYGSSWGVFAYWGQGTLVTVSS
NKp46-2: “H2” heavy chain variable region
(SEQ ID NO: 203)
QVQLQESGPGLVKPSQTLSLTCTVSGYSISSDYAWNWIRQPPGKGLEWMG
YITYSGSTSYNPSLESRITISRDTSKNQFSLKLSSVTAADTAVYYCARGG
YYGSSWGVFAYWGQGTLVTVSS
NKp46-2: “H3” heavy chain variable region
(SEQ ID NO: 204)
QVQLQESGPGLVKPSQTLSLTCTVSGYSITSDYAWNWIRQPPGKGLEWMG
YITYSGSTSYNPSLESRITISRDTSKNQFSLKLSSVTAADTAVYYCARGG
YYGSSWGVFAYWGQGTLVTVSS
NKp46-2: “L1” light chain variable region
(SEQ ID NO: 205)
DIQMTQSPSSLSASVGDRVTITCRVSENIYSYLAWYQQKPGKAPKLLVYN
AKTLAEGVPSRFSGSGSGTDFTLTISSLQPEDFATYYCQHHYGTPWTFGG
GTKVEIK

Antibody NKp46-3

Based on 3D modelling studies, different heavy and light chain variable regions were designed that included NKp46-3 CDRs and human frameworks, produced as human IgG1 antibodies, and tested for binding to cynomolgus NKp46. Three combinations of heavy and light chains were able to bind to both human and cynomolgus NKp46: the heavy chain variable regions “H1”, “H3 and “H4”, in each case combined with the light chain “L1”. Interestingly, the H3L1 furthermore had an improved binding affinity compared to the parental NKp46-3 antibody having the VH and VL of SEQ ID NOS: 7 and 8. These cross-binding variable regions included, for the heavy chain variable region: the NKp46-3 heavy chain CDRs (shown below, underlined), human IGHV1-69*02 gene framework 1, 2 and 3 regions and a human IGHJ6*01 gene framework 4 region. The light chain variable region: the NKp46-3 light chain CDRs (shown below, underlined), framework 1, 2 and 3 regions created by a mosaic approach using FR1 and FR2 from IGKV3-15 and FR3 from IGKV3-11, and a human IGKJ2*01 gene framework 4 region. CDRs were chosen according to Kabat numbering. The L1, H1, H3 and H4 chains had the specific amino acid substitutions (shown in bold and underlining below). L1 had a lysine at Kabat light chain residue 49. H1 had a tyrosine at Kabat heavy chain residue 27. H3 additionally had a isoleucine at Kabat residue 48 and an alanine at Kabat residue 67. H4 additionally had a leucine at Kabat residue 69.

NKp46-3: “H1” heavy chain variable region
(SEQ ID NO: 206)
QVQLVQSGAEVKKPGSSVKVSCKASGYTFSEYTMHWVRQAPGQGLEWMGG
ISPNIGGTSYNQKFKGRVTITADKSTSTAYMELSSLRSEDTAVYYCARRG
GSFDYWGQGTTVTVSS
NKp46-3: “H3” heavy chain variable region
(SEQ ID NO: 207)
QVQLVQSGAEVKKPGSSVKVSCKASGYTFSEYTMHWVRQAPGQGLEWIGG
ISPNIGGTSYNQKFKGRATITADKSTSTAYMELSSLRSEDTAVYYCARRG
GSFDYWGQGTTVTVSS
NKp46-3: “H4” heavy chain variable region
(SEQ ID NO: 208)
QVQLVQSGAEVKKPGSSVKVSCKASGYTFSEYTMHWVRQAPGQGLEWIGG
ISPNIGGTSYNQKFKGRATLTADKSTSTAYMELSSLRSEDTAVYYCARRG
GSFDYWGQGTTVTVSS
NKp46-3: “L1” light chain variable region
(SEQ ID NO: 209)
EIVMTQSPATLSVSPGERATLSCRASQSISDYLHWYQQKPGQAPRLLIKY
ASQSISGIPARFSGSGSGTDFTLTISSLEPEDFAVYYCQNGHSFPLTFGQ
GTKLEIK

Antibody NKp46-4

Based on 3D modelling studies, different heavy and light chain variable regions were designed that included NKp46-4 CDRs and human frameworks, produced as human IgG1 antibodies, and tested for binding to cynomolgus NKp46. The heavy chain variable region “H1” combined with the light chain “L2” was able to bind to both human and cynomolgus NKp46 as well as the parental antibody. Two other antigen binding regions (one composed of “L2” and “H2”, and one composed of “L2” and “H3”) were able to bind cynomolgus NKp46 at intermediate levels, although with an affinity that was 10-fold lower that for human NKp46. These cross-binding variable regions included, for the heavy chain variable region: the NKp46-4 heavy chain CDRs (shown below, underlined), human framework 1, 2 and 3 regions designed using a mosaic approach using FR1 from IGHV1646,0 FR3 from IGHV1-69, and a common FR2 (IGHV1-46 and IGHV1-69 share the same FR2), and a human IGHJ6*01 gene framework 4 region. The light chain variable region: the NKp46-4 light chain CDRs (shown below, underlined), framework 1, 2 and 3 regions from IGKV1-NL1, and a human IGKJ4*01 gene framework 4 region. CDRs were chosen according to Kabat numbering. The L2, H1, H2 and H3 chains had the specific amino acid substitutions (shown in bold and underlining below). L2 had a phenylalanine at Kabat light chain residue 36 and a valine at Kabat residue 48. H1 had a threonine at Kabat heavy chain residue 30 and an isoleucine at Kabat residue 48 and a valine at Kabat residue 93. H2 additionally had a threonine at Kabat residue 67. H3 additionally had a leucine at Kabat residue 69.

NKp46-4: “H1” heavy chain variable region
(SEQ ID NO: 210)
QVQLVQSGAEVKKPGASVKVSCKASGYTFTSFTMHWVRQAPGQGLEWIGY
INPSSGYTEYNQKFKDRVTITADKSTSTAYMELSSLRSEDTAVYYCVRGS
SRGFDYWGQGTLVTVSS
NKp46-4: “H2” heavy chain variable region
(SEQ ID NO: 211)
QVQLVQSGAEVKKPGASVKVSCKASGYTFTSFTMHWVRQAPGQGLEWIGY
INPSSGYTEYNQKFKDRTTITADKSTSTAYMELSSLRSEDTAVYYCVRGS
SRGFDYWGQGTLVTVSS
NKp46-4: “H3” heavy chain variable region
(SEQ ID NO: 212)
QVQLVQSGAEVKKPGASVKVSCKASGYTFTSFTMHWVRQAPGQGLEWIGY
INPSSGYTEYNQKFKDRTTLTADKSTSTAYMELSSLRSEDTAVYYCVRGS
SRGFDYWGQGTLVTVSS
NKp46-4: “L2” light chain variable region
(SEQ ID NO: 213)
DIQMTQSPSSLSASVGDRVTITCRASENIYSNLAWFQQKPGKAPKLLVYA
ATNLADGVPSRFSGSGSGTDYTLTISSLQPEDFATYYCQHFWGTPRTFGG
GTKVEIK

Antibody NKp46-9

Based on 3D modelling studies, different heavy and light chain variable regions were designed that included NKp46-9 CDRs and human frameworks, produced as human IgG1 antibodies, and tested for binding to cynomolgus NKp46. Heavy chain variable region “H2”, when combined with either light chain variable region “L1” or “L2”, and heavy chain variable region “H3” when combined with either light chain variable region “L1” or “L2”, were able to bind to both human and cynomolgus NKp46 with greater affinity that the parental antibody (having the VH and VL of SEQ ID NOS: 13 and 14. The “H1” chain was not able to bind cynomolgus NKp46 with any of the light chains tested but bound well to human NKp46. These cross-binding variable regions included, for the heavy chain variable region: the NKp46-9 heavy chain CDRs (shown below, underlined), human framework 1, 2 and 3 regions from IGHV4-30-4*01, and a human IGHJ6*01 gene framework 4 region. The light chain variable region: the NKp46-9 light chain CDRs (shown below, underlined), framework 1, 2 and 3 regions from IGKV1-39*01, and a human IGKJ2*01 gene framework 4 region. CDRs were chosen according to Kabat numbering. The L1, L2, H1, H2 and H3 chains had the specific amino acid substitutions (shown in bold and underlining below). L1 had a cysteine at Kabat light chain residue 36. L2 additionally had a valine at Kabat residue 48. H1 had an arginine at Kabat heavy chain residue 71. H2 additionally had a tyrosine at Kabat residue 27. H3 additionally had a methionine at Kabat residue 48 and an isoleucine at Kabat residue 67.

NKp46-9: “H1” heavy chain variable region
(SEQ ID NO: 214)
QVQLQESGPGLVKPSQTLSLTCTVSGGSISSDYAWNWIRQPPGKGLEWIG
YITYSGSTNYNPSLKSRVTISRDTSKNQFSLKLSSVTAADTAVYYCARCW
DYALYAMDCWGQGTTVTVSS
NKp46-9: “H2” heavy chain variable region
(SEQ ID NO: 215)
QVQLQESGPGLVKPSQTLSLTCTVSGYSISSDYAWNWIRQPPGKGLEWIG
YITYSGSTNYNPSLKSRVTISRDTSKNQFSLKLSSVTAADTAVYYCARCW
DYALYAMDCWGQGTTVTVSS
NKp46-9: “H3” heavy chain variable region
(SEQ ID NO: 216)
QVQLQESGPGLVKPSQTLSLTCTVSGYSISSDYAWNWIRQPPGKGLEWMG
YITYSGSTNYNPSLKSRITISRDTSKNQFSLKLSSVTAADTAVYYCARCW
DYALYAMDCWGQGTTVTVSS
NKp46-9: “L1” light chain variable region
(SEQ ID NO: 217)
DIQMTQSPSSLSASVGDRVTITCRTSENIYSYLAWCQQKPGKAPKLLIYN
AKTLAEGVPSRFSGSGSGTDFTLTISSLQPEDFATYYCQHHYDTPLTFGQ
GTKLEIK
NKp46-9: “L2” light chain variable region
(SEQ ID NO: 218)
DIQMTQSPSSLSASVGDRVTITCRTSENIYSYLAWCQQKPGKAPKLLVYN
AKTLAEGVPSRFSGSGSGTDFTLTISSLQPEDFATYYCQHHYDTPLTFGQ
GTKLEIK

### Example 2

### Identification of a Bispecific Antibody Format that Binds FcRn but not FcγR for Targeting Effector Cell Receptors

The aim of this experiment was to develop a new bispecific protein format that places an Fc domain on a polypeptide together with an anti-NKp46 binding domain and an anti-target antigen binding domain. The bispecific protein binds to NKp46 monovalently via its anti-NKp46 binding domain. The monomeric Fc domain retains at least partial binding to the human neonatal Fc receptor (FcRn), yet does not substantially bind human CD16 and/or other human Fcγ receptors. Consequently, the bispecific protein will not induce Fcγ-mediated (e.g. CD16-mediated) target cell lysis.

### Example 2-1 Construction and Binding Analysis of Anti-CD19-IgG1-Fcmono-Anti-CD3

Since no anti-NKp46 bispecific antibody has been produced that could indicate whether such a protein could be functional, CD3 was used as a model antigen in place of NKp46 in order to investigate the functionality of a new monovalent bispecific protein format prior to targeting NK cells via NKp46.

A bispecific Fc-based on a scFv specific for tumor antigen CD19 (anti-CD19 scFv) and a scFV specific for activating receptor CD3 on a T cell (anti-CD3 scFv) was used to assess FcRn binding and CD19-binding functions of a new monomeric bispecific polypeptide format. The domain arrangement of the final polypeptide is referred to as the “F1” format (the star in the CH2 domain indicates an optional N297S mutation, not included in the polypeptide tested here).

A bispecific monomeric Fc-containing polypeptide was constructed based on an scFv specific for the tumor antigen CD19 (anti-CD19 scFv) and an scFV specific for an activating receptor CD3 on a T cell (anti-CD3 scFv). The CH3 domain incorporated the mutations (EU numbering) L351K, T366S, P395V, F405R, T407A and K409Y. The polypeptide has domains arranged as follows: anti-CD19-CH2-CH3-anti-CD3. DNA sequence coding for a CH3/VH linker peptide having the amino acid sequence STGS was designed in order to insert a specific Sall restriction site at the CH3-VH junction.

The CH3 domain incorporated the mutations (EU numbering) L351K, T366S, P395V, F405R, T407A and K409Y. The CH2 domain was a wild-type CH2. DNA and amino acid sequences for the monomeric CH2-CH3 Fc portion and the anti-CD19 are shown below.

The light chain and heavy chain DNA and amino acid sequences corresponding to the anti-CD19 scFv were as follows:

| Sequence | SEQ ID NO |
|---|---|
| Anti-CD19-VK DNA | 113 |
| Anti-CD19-VK amino acid | 114 |
| Anti-CD19-VH DNA | 115 |
| Anti-CD19-VH amino acid | 116 |

The DNA sequences for the monomeric CH2-CH3 Fc portion and final bispecific IgG1-Fcmono polypeptide (the last K was removed in that construct) is shown in SEQ ID NO: 117. The amino acid sequence is shown in SEQ ID NO: 2. The Anti-CD19-F1-Anti-CD3 complete sequence (mature protein) is shown in SEQ ID NO: 118.

### Cloning and Production of the Recombinant Proteins

Coding sequences were generated by direct synthesis and/or by PCR. PCR were performed using the PrimeSTAR MAX DNA polymerase (Takara, #R045A) and PCR products were purified from 1% agarose gel using the NucleoSpin gel and PCR clean-up kit (Macherey-Nagel, #740609.250). Once purified the PCR product were quantified prior to the In-Fusion ligation reaction performed as described in the manufacturer's protocol (ClonTech, #ST0345). The plasmids were obtained after a miniprep preparation run on an EVO200 (Tecan) using the Nucleospin 96 plasmid kit (Macherey-Nagel, #740625.4). Plasmids were then sequenced for sequences confirmation before to transfecting the CHO cell line.

CHO cells were grown in the CD-CHO medium (Invitrogen) complemented with phenol red and 6 mM GlutaMax. The day before the transfection, cells are counted and seeded at 175.000 cells/ml. For the transfection, cells (200.000 cells/transfection) are prepared as described in the AMAXA SF cell line kit (AMAXA, #V4XC-2032) and nucleofected using the DS137 protocol with the Nucleofector 4D device. All the tranfections were performed using 300 ng of verified plasmids. After transfection, cells are seeded into 24 well plates in pre-warmed culture medium. After 24H, hygromycine B was added in the culture medium (200 μg/ml). Protein expression is monitored after one week in culture. Cells expressing the proteins are then sub-cloned to obtain the best producers. Sub-cloning was performed using 96 flat-bottom well plates in which the cells are seeded at one cell per well into 200 ul of culture medium complemented with 200 ug/ml of hygromycine B. Cells were left for three weeks before to test the clone's productivity.

Recombinant proteins which contain a IgG1-Fc fragment are purified using Protein-A beads (-rProteinA Sepharose fast flow, GE Healthcare, ref .: 17-1279-03). Briefly, cell culture supernatants were concentrated, clarified by centrifugation and injected onto Protein-A columns to capture the recombinant Fc containing proteins. Proteins were eluted at acidic pH (citric acid 0.1M pH3), immediately neutralized using TRIS-HCL pH8.5 and dialyzed against 1× PBS. Recombinant scFv which contain a “six his” tag were purified by affinity chromatography using Cobalt resin. Other recombinant scFv were purified by size exclusion chromatography (SEC).

### Example 2-2: Binding Analysis of Anti-CD19-IgG1-Fcmono-Anti-CD3 to B221, JURKAT, HUT78 and CHO Cell Lines

Cells were harvested and stained with the cell supernatant of the anti-CD19-F1-anti-CD3 producing cells during 1 H at 4° C. After two washes in staining buffer (PBS1×/BSA 0.2%/EDTA 2 mM), cells were stained for 30 min at 4° C. with goat anti-human (Fc)-PE antibody (IM0550 Beckman Coulter-1/200). After two washes, stainings were acquired on a BD FACS Canto Il and analyzed using the FlowJo software.

CD3 and CD19 expression were also controlled by flow cytometry: Cells were harvested and stained in PBS1×/BSA 0.2%/EDTA 2 mM buffer during 30 min at 4° C. using 5 μl of the anti-CD3-APC and 5 μl of the anti-CD19-FITC antibodies. After two washes, stainings were acquired on a BD FACS Canto II and analyzed using the FlowJo software.

The Anti-CD19-F1-Anti-CD3 protein binds to the CD3 cell lines (HUT78 and JURKAT cell lines) and the CD19 cell line (B221 cell line) but not to the CHO cell line used as a negative control.

### Example 2-3

### T- and B-Cell Aggregation by Purified Anti-CD19-F1-Anti-CD3

Purified Anti-CD19-F1-Anti-CD3 was tested in a T/B cell aggregation assay to evaluate whether the antibody is functional in bringing together CD19 and CD3 expressing cells.

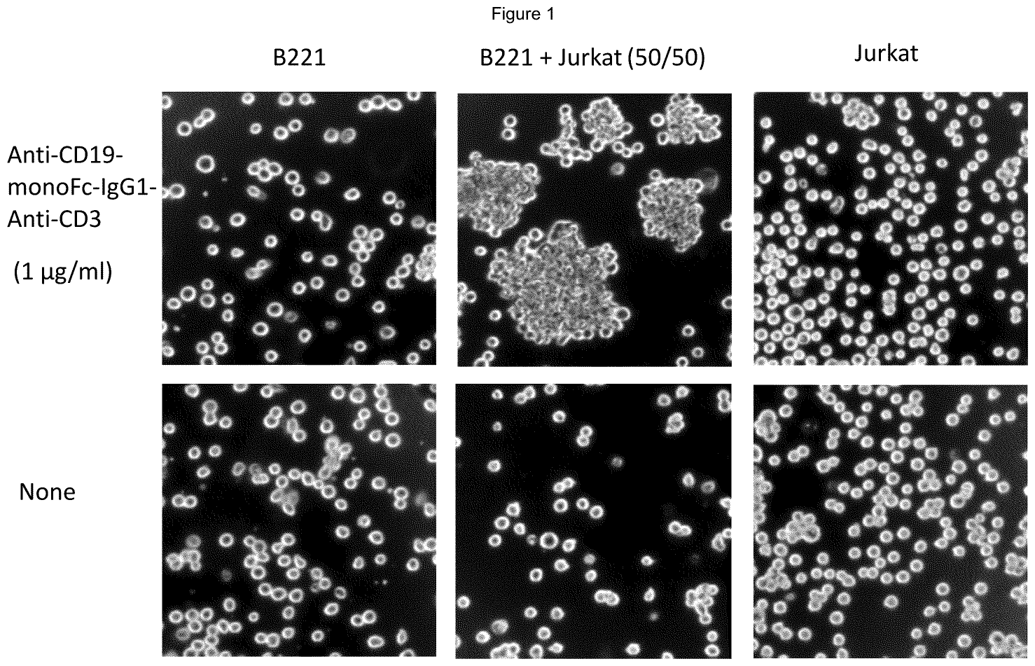

Results are shown in FIG. 1. The top panel shows that Anti-CD19-F1-Anti-CD3 does not cause aggregation in the presence of B221 (CD19) or JURKAT (CD3) cell lines, but it does cause aggregation of cells when both B221 and JURKAT cells are co-incubated, illustrating that the bispecific antibody is functional. The lower panel shows control without antibody.

### Example 2-4

### Binding of Bispecific Monomeric Fc Polypeptide to FcRn

Affinity study by Surface Plasmon Resonance (SPR)

### Biacore T100 General Procedure and Reagents

SPR measurements were performed on a Biacore T100 apparatus (Biacore GE Healthcare) at 25° C. In all Biacore experiments Acetate Buffer (50 mM Acetate pH5.6, 150 mM NaCl, 0.1% surfactant p20) and HBS-EP+(Biacore GE Healthcare) served as running buffer and regeneration buffer respectively. Sensorgrams were analyzed with Biacore T100 Evaluation software. Recombinant mouse FcRn was purchase from R&D Systems.

### Immobilization of FcRn

Recombinant FcRn proteins were immobilized covalently to carboxyl groups in the dextran layer on a Sensor Chip CM5. The chip surface was activated with EDC/NHS (N-ethyl-N′-(3-dimethylaminopropyl) carbodiimidehydrochloride and N-hydroxysuccinimide (Biacore GE Healthcare)). FcRn proteins were diluted to 10 ug/ml in coupling buffer (10 mM acetate, pH 5.6) and injected until the appropriate immobilization level was reached (i.e. 2500 RU). Deactivation of the remaining activated groups was performed using 100 mM ethanolamine pH 8 (Biacore GE Healthcare).

### Affinity Study

Monovalent affinity study was done following the Single Cycle Kinetic (SCK) protocol. Five serial dilutions of soluble analytes (antibodies and bi-specific molecules) ranging from 41.5 to 660 nM were injected over the FcRn (without regeneration) and allowed to dissociate for 10 min before regeneration. For each analyte, the entire sensorgram was fitted using the 1:1 SCK binding model.

### Results

Anti-CD19-F1-Anti-CD3 having its CH2-CH3 domains placed between two antigen binding domains, here two scFv, was evaluated to assess whether such bispecific monomeric Fc protein could retain binding to FcRn and thereby have improved in vivo half-lives compared to convention bispecific antibodies. Results showed that FcRn binding was retained, the model suggesting 1:1 ratio (1 FcRn for each monomeric Fc) instead of 2:1 ration (2 FcRn for each antibody) for a regular IgG.

Affinity was evaluated using SPR, in comparison to a chimeric full length antibody having human IgG1 constant regions. The monomeric Fc retained significant monomeric binding to FcRn (monomeric Fc: affinity of KD=194 nM; full length antibody with bivalent binding: avidity of KD=15.4 nM).

### Example 3

### Construction of Anti-CD19 x Anti-NKp46 Bispecific Monomeric Fc Domain Polypeptides

It was unknown what activating receptors on NK cells would contribute to the lysis of target cells, and moreover since anti-NKp46 antibodies may block NKp46, it was further unknown whether cytotoxicity could be mediated by NKp46. We therefore investigated whether the bispecific protein format could induce NKp46 triggering, and whether it would induce NKp46 agonism in the absence of target cells, which could lead to inappropriate NK activation distant from the target and/or decreased overall activity toward target cells.

A new bispecific protein format was developed as a single chain protein which binds to FcRn but not FcγR. Additionally, multimeric proteins that comprise two or three polypeptide chains, wherein the Fc domain remains monomeric, were developed that are compatible for use with antibody variable regions that do not maintain binding to their target when converted to scFv format. The latter formats can be used conveniently for antibody screening; by incorporating at least one binding region as a F(ab) structure, any anti-target (e.g. anti-tumor) antibody variable region can be directly expressed in a bispecific construct as the F(ab) format within the bispecific protein and tested, irrespective of whether the antibody would retain binding as an scFv, thereby simplifying screening and enhancing the number of antibodies available. These formats in which the Fc domain remains monomeric have the advantage of maintaining maximum conformational flexibility and as shown infra may permit optimal binding to NKp46 or target antigens.

Different constructs were made for use in the preparation of bispecific antibodies using the variable domains from the scFv specific for tumor antigen CD19 described in Example 2-1, and different variable regions from antibodies specific for the NKp46 receptor identified in Example 1. A construct was also made using as the anti-NKp46 the variable regions from a commercially available antibody Bab281 (mIgG1, available commercially from Beckman Coulter, Inc. (Brea, CA, USA) (see also Pessino et al, J. Exp. Med, 1998, 188 (5): 953-960 and Sivori et al, Eur J Immunol, 1999. 29:1656-1666) specific for the NKp46 receptor.

In order for the Fc domain to remain monomeric in single chain polypeptides or in multimers in which only one chain had an Fc domain, CH3-CH3 dimerization was prevented through two different strategies: (1) through the use of CH3 domain incorporating specific mutations (EU numbering), i.e., L351K, T366S, P395V, F405R, T407A and K409Y; or (2) through the use of a tandem CH3 domain in which the tandem CH3 domains are separated by a flexible linker associated with one another, which prevents interchain CH3-CH3 dimerization. The DNA and amino acid sequences for the monomeric CH2-CH3 Fc portion containing the above-identified point mutations were the same as in Example 2-1. The DNA and amino acid sequences for the monomeric CH2-CH3-linker-CH3 Fc portion with tandem CH3 domains are shown in FIGS. 2A-2D.

The light chain and heavy chain DNA and amino acid sequences for the anti-CD19 scFv were also the same as in Example 2-1. Proteins were cloned, produced and purified as in Example 2-1. Shown below are the light chain and heavy chain DNA and amino acid sequences for different anti-NKp46 scFvs.

TABLE 1
Amino acid sequences of different anti-NKp46 scFvs

| scFv | |
|---|---|
| anti-NKp46 | scFV sequence (VHVK)/-stop |
| NKp46-1 | STGSQVQLQQSGPELVKPGASVKMSCKASGYTFTDYVINWGKQRSGQ |
| | GLEWIGEIYPGSGTNYYNEKFKAKATLTADKSSNIAYMQLSSLTSEDSAV |
| | YFCARRGRYGLYAMDYWGQGTSVTVSSVEGGSGGSGGSGGSGGVDD |
| | IQMTQTTSSLSASLGDRVTISCRASQDISNYLNWYQQKPDGTVKLLIYYT |
| | SRLHSGVPSRFSGSGSGTDYSLTINNLEQEDIATYFCQQGNTRPWTFG |
| | GGTKLEIK- (SEQ ID NO: 119) |
| NKp46-2 | STGSEVQLQESGPGLVKPSQSLSLTCTVTGYSITSDYAWNWIRQFPGNK |
| | LEWMGYITYSGSTSYNPSLESRISITRDTSTNQFFLQLNSVTTEDTATYY |
| | CARGGYYGSSWGVFAYWGQGTLVTVSAVEGGSGGSGGSGGSGGVD |
| | DIQMTQSPASLSASVGETVTITCRVSENIYSYLAWYQQKQGKSPQLLVY |
| | NAKTLAEGVPSRFSGSGSGTQFSLKINSLQPEDFGSYYCQHHYGTPWT |
| | FGGGTKLEIK- (SEQ ID NO: 120) |
| NKp46-3 | STGSEVQLQQSGPELVKPGASVKISCKTSGYTFTEYTMHWVKQSHGKS |
| | LEWIGGISPNIGGTSYNQKFKGKATLTVDKSSSTAYMELRSLTSEDSAVY |
| | YCARRGGSFDYWGQGTTLTVSSVEGGSGGSGGSGGSGGVDDIVMTQ |
| | SPATLSVTPGDRVSLSCRASQSISDYLHWYQQKSHESPRLLIKYASQSIS |
| | GIPSRFSGSGSGSDFTLSINSVEPEDVGVYYCQNGHSFPLTFGAGTKLE |
| | LK- (SEQ ID NO: 121) |
| NKp46-4 | STGSQVQLQQSAVELARPGASVKMSCKASGYTFTSFTMHWVKQRPGQ |
| | GLEWIGYINPSSGYTEYNQKFKDKTTLTADKSSSTAYMQLDSLTSDDSA |
| | VYYCVRGSSRGFDYWGQGTLVTVSAVEGGSGGSGGSGGSGGVDDIQ |
| | MIQSPASLSVSVGETVTITCRASENIYSNLAWFQQKQGKSPQLLVYAATN |
| | LADGVPSRFSGSGSGTQYSLKINSLQSEDFGIYYCQHFWGTPRTFGGG |
| | TKLEIK- (SEQ ID NO: 122) |
| NKp46-6 | STGSQVQLQQPGSVLVRPGASVKLSCKASGYTFTSSWMHWAKQRPGQ |
| | GLEWIGHIHPNSGISNYNEKFKGKATLTVDTSSSTAYVDLSSLTSEDSAV |
| | YYCARGGRFDDWGAGTTVTVSSVEGGSGGSGGSGGSGGVDDIVMTQ |
| | SPATLSVTPGDRVSLSCRASQSISDYLHWYQQKSHESPRLLIKYASQSIS |
| | GIPSRFSGSGSGSDFTLSINSVEPEDVGVYYCQNGHSFLMYTFGGGTKL |
| | EIK- (SEQ ID NO: 123) |
| NKp46-9 | STGSDVQLQESGPGLVKPSQSLSLTCTVTGYSITSDYAWNWIRQFPGNK |
| | LEWMGYITYSGSTNYNPSLKSRISITRDTSKNQFFLQLNSVTTEDTATYY |
| | CARCWDYALYAMDCWGQGTSVTVSSVEGGSGGSGGSGGSGGVDDIQ |
| | MTQSPASLSASVGETVTITCRTSENIYSYLAWCQQKQGKSPQLLVYNAK |
| | TLAEGVPSRFSGSGSGTHFSLKINSLQPEDFGIYYCQHHYDTPLTFGAG |
| | TKLELK- (SEQ ID NO: 124) |
| Bab281 | STGSQIQLVQSGPELQKPGETVKISCKASGYTFTNYGMNWVKQAPGKG |
| | LKWMGWINTNTGEPTYAEEFKGRFAFSLETSASTAYLQINNLKNEDTAT |
| | YFCARDYLYYFDYWGQGTTLTVSSVEGGSGGSGGSGGSGGVDNIVMT |
| | QSPKSMSMSVGERVTLTCKASENVVTYVSWYQQKPEQSPKLLIYGASN |
| | RYTGVPDRFTGSGSATDFTLTISSVQAEDLADYHCGQGYSYPYTFGGG |
| | TKLEIK- (SEQ ID NO: 125) |

TABLE 2A
DNA sequences corresponding to different anti-NKp46 scFvs

| scFv anti-NKp46 | scFV sequences |
|---|---|
| NKp46-1 | SEQ ID NO: 126 |
| NKp46-2 | SEQ ID NO: 127 |
| NKp46-3 | SEQ ID NO: 128 |
| NKp46-4 | SEQ ID NO: 129 |
| NKp46-6 | SEQ ID NO: 130 |
| NKp46-9 | SEQ ID NO: 131 |
| Bab281 | SEQ ID NO: 132 |

Format 1 (F1) (Anti-CD19-IgG1-Fcmono-Anti-NKp46 (scFv))

The domain structure of Format 1 (F1) is shown in FIG. 2A. A bispecific Fc-containing polypeptide was constructed based on a scFv specific for the tumor antigen CD19 (anti-CD19 scFv) and an scFV specific for the NKp46 receptor. The polypeptide is a single chain polypeptide having domains arranged (N- to C-termini) as follows:

(V_{K}-V_{H})anti-CD19-CH2-CH3-(V_{H}-V_{K})^{anti-NKp46}

A DNA sequence coding for a CH3/VH linker peptide having the amino acid sequence STGS was designed in order to insert a specific Sall restriction site at the CH3-V_{H} junction. The domain arrangement of the final polypeptide in shown in FIG. 2 (the star in the CH2 domain indicates an optional N297S mutation), where the anti-CD3 scFv is replaced by an anti-NKp46 scFv. The (V_{K}-V_{H}) units include a linker between the V_{H} and V_{K} domains. Proteins were cloned, produced and purified as in Example 2-1. The amino acid sequences of the bispecific polypeptides (complete sequence (mature protein)) are shown in the corresponding SEQ ID NOS listed in the Table 2B below.

TABLE 2B

| Sequence | SEQ ID NO |
|---|---|
| CD19-F1-NKp46-1 | 133 |
| CD19-F1-NKp46-2 | 134 |
| CD19-F1-NKp46-3 | 135 |
| CD19-F1-NKp46-4 | 136 |
| CD19-F1-NKp46-6 | 137 |
| CD19-F1-NKp46-9 | 138 |
| CD19-F1-Bab281 | 139 |

### Format 2 (F2): CD19-F2-NKp46-3

The domain structure of F2 polypeptides is shown in FIG. 2A. The DNA and amino acid sequences for the monomeric CH2-CH3 Fc portion were as in Example 2-1 and it similarly contains CH3 domain mutations (the mutations (EU numbering) L351K, T366S, P395V, F405R, T407A and K409Y). The heterodimer is made up of:

(1) a first (central) polypeptide chain having domains arranged as follows (N- to C-termini):

(V_{K}-V_{H})^{anti-CD19}-CH2-CH3-V_{H}^{anti-NKp46}-CH1

and

(2) a second polypeptide chain having domains arranged as follows (N- to C-termini): VK^{anti-NKp46}-CK.

The (V_{K}-V_{H}) unit was made up of a V_{H} domain, a linker and a V_{K}unit (i.e. an scFv). As with other formats of the inventive bispecific polypeptides, the DNA sequence coded for a CH3/VH linker peptide having the amino acid sequence STGS designed in order to insert a specific Sall restriction site at the CH3-VH junction. Proteins were cloned, produced and purified as in Example 2-1. The amino acid sequences for the first and second chains of the F2 protein are shown in SEQ ID NO: 140 and 141.

### Format 3 (F3): CD19-F3-NKp46-3

The domain structure of F3 polypeptides is shown in FIG. 2A. The DNA and amino acid sequences for the CH2-CH3 Fc portion comprised a tandem CH3 domain in which the two CH3 domains on the same polypeptide chain associated with one another, thereby preventing dimerization between different bispecific proteins.

The single chain polypeptide has domains arranged (N- to C- termini) as follows:

(V_{K}-V_{H})^{anti-CD19}-CH2-CH3-CH3-(V_{H}-V_{K})^{anti-NKp46}.

The (V_{K}-V_{H}) units were made up of a V_{H} domain, a linker and a V_{K} unit (scFv). Proteins were cloned, produced and purified as in Example 2-1. Bispecific protein was purified from cell culture supernatant by affinity chromatography using prot-A beads and analyzed and purified by SEC. The protein showed a high production yield of 3.4 mg/L and the purified proteins exhibited a simple SEC profile. The amino acid sequence for the F3 protein is shown in SEQ ID NO: 142.

### Format 4 (F4): CD19-F4-NKp46-3

The domain structure of F4 polypeptides is shown in FIG. 2A. The DNA and amino acid sequences for the CH2-CH3 Fc portion comprised a tandem CH3 domain as in Format F3, and additionally comprise a N297S mutation which prevents N-linked glycosylation and abolishes FcγR binding. Proteins were cloned, produced and purified as in Example 2-1. Bispecific proteins was purified from cell culture supernatant by affinity chromatography using prot-A beads and analyzed and purified by SEC. The protein showed a good production yield of 1 mg/L and the purified proteins exhibited a simple SEC profile. The amino acid sequence for the F4 protein with NKp46-3 variable domains is shown in SEQ ID NO: 143.

### Format 8 (F8)

The domain structure of F8 polypeptides is shown in FIG. 2B. The DNA and amino acid sequences for the monomeric CH2-CH3 Fc portion were as in Format F2 and it similarly contains CH3 domain mutations (the mutations (EU numbering) L351K, T366S, P395V, F405R, T407A and K409Y), as well as a N297S mutation which prevents N-linked glycosylation and moreover abolishes FcγR binding. Three variants of F8 proteins were produced: (a) one wherein the cysteine residues in the hinge region were left intact (wild-type, referred to as F8A), (b) a second wherein the cysteine residues in the hinge region were replaced by serine residues (F8B), and (c) a third including a linker sequence GGGSS replacing residues DKTHTCPPCP in the hinge (F8C). Variants F8B and F8C provided production advantages as these versions avoided the formation of homodimers of the central chain. This heterotrimer is made up of:

(1) a first (central) polypeptide chain having domains arranged as follows (N- to C-termini):

V_{H}^{anti-CD19}-CH1-CH2-CH3-VH^{anti-NKp46}-C_{K};

and

(2) a second polypeptide chain having domains arranged as follows (N- to C-termini):

VK^{anti-NKp46}-CH1;

and

(3) a third polypeptide chain having domains arranged as follows (N- to C- termini): V_{K}^{anti-CD19}-C_{K}.

Proteins were cloned, produced and purified as in Example 2-1. Bispecific proteins was purified from cell culture supernatant by affinity chromatography using prot-A beads and analyzed and purified by SEC. The protein showed a high production yield of 3.7 mg/L (F8C) and the purified proteins again exhibited a simple SEC profile. The amino acid sequences of the three chains of the F8 protein (C variant) with NKp46-3 variable regions are shown in SEQ ID NOS: 144, 145 and 146.

### Format 9 (F9): CD19-F9-NKp46-3

The F9 polypeptide is a trimeric polypeptide having a central polypeptide chain and two polypeptide chains each of which associate with the central chain via CH1-C_{K} dimerization. The domain structure of the trimeric F9 protein is shown in FIG. 2B, wherein the bonds between the CH1 and C_{K} domains are interchain disulfide bonds. The two antigen binding domains have a F(ab) structure permitting the use of these antibodies irrespective of whether they remain functional in a scFv format. The DNA and amino acid sequences for the CH2-CH3 Fc portion comprise a tandem CH3 domain as in Format F4 and comprise a CH2 domain comprising a N297S substitution. Three variants of F9 proteins were produced: (a) a first wherein the cysteine residues in the hinge region left intact (wild-type, referred to as F9A), (b) a second wherein the cysteine residues in the hinge region were replaced by serine residues (F9B), and (c) a third containing a linker sequence GGGSS which replaces residues DKTHTCPPCP in the hinge (F9C). Variants F9B and F9C provided advantages in production by avoiding the formation of homodimers of the central chain. The heterotrimer is made up of:

(1) a first (central) polypeptide chain having domains arranged as follows (N- to C-termini):

V_{H}^{anti-CD19}-CH1-CH2-CH3-CH3-V_{H}^{anti-NKp46}-Cκ

and

(2) a second polypeptide chain having domains arranged as follows (N- to C-termini):

V_{K}^{anti-NKp46}-CH1

and

(3) a third polypeptide chain having domains arranged as follows (N- to C- termini):

V_{K}^{anti-CD19}-C_{K}.

Proteins were cloned, produced and purified as in Example 2-1. Bispecific proteins was purified from cell culture supernatant by affinity chromatography using prot-A beads and analyzed and purified by SEC. The protein showed a high production yield of 8.7 mg/L (F9A) and 3.0 mg/L (F9B), and the purified proteins again exhibited a simple SEC profile.

The amino acid sequences of the three chains of the F9 protein variant F9A are shown in the SEQ ID NOS: 147, 148 and 149. The amino acid sequences of the three chains of the F9 protein variant F9B are shown in the SEQ ID NOS: 150, 151 and 152. The amino acid sequences of the three chains of the F9 protein variant F9C are shown in the SEQ ID NOS: 153, 154 and 155.

### Format 10 (F10): CD19-F10-NKp46-3

The F10 polypeptide is a dimeric protein having a central polypeptide chain and a second polypeptide chain which associates with the central chain via CH1-C_{K} dimerization. The domain structure of the dimeric F10 protein is shown in FIG. 2B wherein the bonds between the CH1 and C_{K} domains are interchain disulfide bonds. One of the two antigen binding domains has a Fab structure, and the other is a scFv. The DNA and amino acid sequences for the CH2-CH3 Fc portion comprise a tandem CH3 domain as shown in Format F4 and comprise a CH2 domain containing a N297S substitution. Three variants of F10 proteins were also produced: (a) a first wherein the cysteine residues in the hinge region were left intact (wild-type, referred to as F10A), (b) a second wherein the cysteine residues in the hinge region were replaced by serine residues (F10B), and (c) a third containing a linker sequence GGGSS replacing residues DKTHTCPPCP in the hinge (F10C). Variants F10B and F10C provided advantages in production as they avoid the formation of homodimers of the central chain. The (V_{K}-V_{H}) unit was made up of a V_{H} domain, a linker and a V_{K} unit (scFv). The heterodimer is made up of:

(1) a first (central) polypeptide chain having domains arranged as follows (N- to C-termini):

V_{H}^{anti-CD19}-CH1-CH2-CH3-CH3-(V_{H}-V_{K})^{anti-NKp46}

and

(2) a second polypeptide chain having domains arranged as follows (N- to C-termini):

V_{K}^{anti-CD19}-C_{K}.

These proteins were cloned, produced and purified as in Example 2-1. Bispecific proteins was purified from cell culture supernatant by affinity chromatography using prot-A beads and analyzed and purified by SEC. The protein showed a good production yield of 2 mg/L (F10A) and the purified proteins again exhibited a simple SEC profile. The amino acid sequences of the two chains of the F10A protein variant are shown in SEQ ID NOS: 156 (second chain) and 157 (first chain). The amino acid sequences of the two chains of the F10B protein variant are shown in SEQ ID NOS: 158 (second chain) and 159 (first chain). The amino acid sequences of the two chains of the F10C protein variant are shown in the

SEQ ID NOS: 160 (second chain) and 161 (first chain).

### Format 11 (F11): CD19-F11-NKp46-3

The domain structure of F11 polypeptides is shown in FIG. 2C. The heterodimeric protein is similar to F10 except that the structures of the antigen binding domains are reversed. One of the two antigen binding domains has a Fab-like structure, and the other is a scFv. The heterodimer is made up of:

(1) a first (central) polypeptide chain having domains arranged as follows (N- to C-termini):

(V_{K}-V_{H})^{anti-CD19}-CH2-CH3-CH3-VH^{anti-NKp46}-C_{K}

and

(2) a second polypeptide chain having domains arranged as follows (N- to C-termini): V_{K}^{anti-NKp46}-CH1.

Proteins were cloned, produced and purified as in Example 2-1. Bispecific proteins was purified from cell culture supernatant by affinity chromatography using prot-A beads and analyzed and purified by SEC. The protein showed a good production yield of 2 mg/L and the purified proteins similarly exhibited a simple SEC profile. The amino acid sequences of the two chains of the F11 protein are shown in SEQ ID NO: 162 (chain 1) and SEQ ID NO: 163 (chain 2).

### Format 12 (F12): CD19-F12-NKp46-3

The domain structure of the dimeric F12 polypeptides is shown in FIG. 2C, wherein the bonds between the CH1 and C_{K} domains are disulfide bonds. The heterodimeric protein is similar to F11 but the CH1 and C_{K} domains within the F(ab) structure are inversed. The heterodimer is made up of:

(1) a first (central) polypeptide chain having domains arranged as follows (N- to C-termini):

(V_{K}-V_{H})^{anti-CD19}-CH2-CH3-CH3-V_{H}^{anti-NKp46}-CH1

and

(2) a second polypeptide chain having domains arranged as follows (N- to C-termini):

V_{K}^{anti-NKp46}-C_{K}.

Proteins were cloned, produced and purified as in Example 2-1. Bispecific proteins were purified from the cell culture supernatant by affinity chromatography using prot-A beads and analyzed and purified by SEC. The protein showed a good production yield of 2.8 mg/L and the purified proteins similarly exhibited a simple SEC profile. The amino acid sequences of the two chains of the F12 protein are shown in SEQ ID NO: 164 (chain 1) and SEQ ID NO: 165 (chain 2).

### Format 17 (F17): CD19-F17-NKp46-3

The domain structure of the trimeric F17 polypeptides is shown in FIG. 2C, wherein the bonds between the CH1 and C_{K} domains are disulfide bonds. The heterodimeric protein is similar to F9 but the V_{H} and V_{K} domains, and the CH1 and C_{K}, domains within the C-terminal F(ab) structure are each respectively inversed with their partner. The heterotrimer is made up of:

(1) a first (central) polypeptide chain having domains arranged as follows (N- to C-termini):

V_{H}^{anti-CD19}-CH1-CH2-CH3-CH3-V_{K}^{anti-NKp46}-CH1;

and

(2) a second polypeptide chain having domains arranged as follows (N- to C-termini): V_{H}^{anti-NKp46}-C_{K};

and

(3) a third polypeptide chain having domains arranged as follows (N- to C-termini): V_{K}^{anti-CDb 19}-C_{K}.

Additionally, three variants of F17 proteins were produced: (a) a first where the cysteine residues in the hinge region were left intact (wild-type, referred to as F17A), (b) a second wherein the cysteine residues in the hinge region were replaced by serine residues (F10B, and (c) a third containing a linker sequence GGGSS which replaces residues DKTHTCPPCP in the hinge (F17C). Proteins were cloned, produced and purified as in Example 2-1. The amino acid sequences of the three chains of the F17B protein are shown in SEQ ID NOS: 166, 167 and 168.

### Example 4

### Bispecific NKp46 Antibody Formats with Dmeric Fc Domains

New protein constructions with dimeric Fc domains were developed that share many of the advantages of the monomeric Fc domain proteins of Example 3 but bind to FcRn with greater affinity. Different protein formats were produced that either had low or substantially lack of binding to FcγR (including CD16) or which had binding to FcγRs (including CD16), e.g. the binding affinity to human CD16 was within 1-log of that of wild-type human IgG1 antibodies, as assessed by SPR (e.g. see methods of Example 16). The different polypeptide formats were tested and compared to investigate the functionality of heterodimeric proteins comprising a central chain with a (V_{H}-(CH1/C_{K})-CH2-CH3-) unit or a (V_{K}-(CH1 or C_{K})-CH2-CH3-) unit. One of both of the CH3 domains are fused, optionally via intervening amino acid sequences or domains, to a variable domain(s) (a single variable domain that associates with a variable domain on a separated polypeptide chain, a tandem variable domain (e.g., an scFv), or a single variable domain that is capable of binding antigen as a single variable domain). The two chains associate by CH1-C_{K} dimerization to form disulfide linked dimers, or if associated with a third chain, to form trimers.

Different constructs were made for use in the preparation of a bispecific antibody using the variable domains DNA and amino acid sequences derived from the scFv specific for tumor antigen CD19 described in Example 2-1 and different variable regions from antibodies specific for NKp46 identified in Example 1. Proteins were cloned, produced and purified as in Example 2-1. Domains structures are shown in FIGS. 2A-6D.

Format 5 (F5): CD19-F5-NKp46-3

The domain structure of the trimeric F5 polypeptide is shown in FIG. 2D, wherein the interchain bonds between hinge domains (indicated in the figures between CH1/C_{K} and CH2 domains on a chain) and interchain bonds between the CH1 and C_{K} domains are interchain disulfide bonds. The heterotrimer is made up of:

(1) a first (central) polypeptide chain having domains arranged as follows (N- to C-termini):

V_{H}^{anti-CD19}-CH1-CH2-CH3-V_{H}^{anti-NKp46}-C_{K};

and

(2) a second polypeptide chain having domains arranged as follows (N- to C-termini): V_{K}^{anti-CD19}-CK-CH2-CH3;

and

(3) a third polypeptide chain having domains arranged as follows (N- to C- termini):

V_{K}^{anti-NKp46}-CH1.

Proteins were cloned, produced and purified as in Example 2-1. Bispecific proteins was purified from cell culture supernatant by affinity chromatography using prot-A beads and analyzed and purified by SEC. The protein showed a high production yield of 37 mg/L and the purified proteins again exhibited a simple SEC profile. The amino acid sequences of the three polypeptide chains are shown in SEQ ID NOS 169 (second chain), 170 (first chain) and 171 (third chain).

Format 6 (F6): CD19-F6-NKp46-3

The domain structure of heterotrimeric F6 polypeptides is shown in FIG. 2D. The F6 protein is the same as F5, but contains a N297S substitution to avoid N-linked glycosylation. Proteins were cloned, produced and purified as in Example 2-1. Bispecific proteins were purified from cell culture supernatant by affinity chromatography using prot-A beads and analyzed and purified by SEC. The protein showed a high production yield of 12 mg/L and the purified proteins exhibited a simple SEC profile. The amino acid sequences of the three polypeptide chains are shown in SEQ ID NOS: 172 (second chain), 173 (first chain) and 174 (third chain).

Format 7 (F7): CD19-F7-NKp46-3

The domain structure of heterotrimeric F7 polypeptides is shown in FIG. 2D. The F7 protein is the same as F6, except for cysteine to serine substitutions in the CH1 and C_{K} domains that are linked at their C-termini to Fc domains, in order to prevent formation of a minor population of dimeric species of the central chain with the V_{K}^{anti-NKp46}-CH1 chain. Proteins were cloned, produced and purified as in Example 2-1. Bispecific proteins were purified from the cell culture supernatant by affinity chromatography using prot-A beads and analyzed and purified by SEC. The protein showed a high production yield of 11 mg/L and the purified proteins exhibited a simple SEC profile. The amino acid sequences of the three polypeptide chains are shown in SEQ ID NOS: 175 (second chain), 176 (first chain) and 177 (third chain).

### Format 13 (F13): CD19-F13-NKp46-3

The domain structure of the dimeric F13 polypeptide is shown in FIG. 2D, wherein the interchain bonds between hinge domains (indicated between CH1/C_{K} and CH2 domains on a chain) and interchain bonds between the CH1 and C_{K} domains are interchain disulfide bonds. The heterodimer is made up of:

(1) a first (central) polypeptide chain having domains arranged as follows (N- to C-termini):

V_{H}anti-CD19- CH1-CH2-CH3-(V_{H}-V_{K})^{anti-NKp46}

and

(2) a second polypeptide chain having domains arranged as follows (N- to C-termini): V_{K}^{anti-CD19}-C_{K}-CH2-CH3.

The (V_{H}-V_{K}) unit was made up of a V_{H} domain, a linker and a V_{K} unit (scFv).

Proteins were cloned, produced and purified as in Example 2-1. Bispecific proteins were purified from the cell culture supernatant by affinity chromatography using prot-A beads and analyzed and purified by SEC. The protein showed a high production yield of 6.4 mg/L and the purified proteins exhibited a simple SEC profile. The amino acid sequences of the two polypeptide chains are shown in SEQ ID NOS: 178 (second chain) and 179 (first chain).

Format 14 (F14): CD19-F14-NKp46-3

The domain structure of the dimeric F14 polypeptide is shown in FIG. 2E. The F14 polypeptide is a dimeric polypeptide which shares the structure of the F13 format, but instead of a wild-type Fc domain (CH2-CH3), the F14 bispecific format has CH2 domain mutations N297S to abolish N-linked glycosylation. Proteins were cloned, produced and purified as in Example 2-1. Bispecific proteins were purified from cell culture supernatant by affinity chromatography using prot-A beads and analyzed and purified by SEC. The protein showed a high production yield of 2.4 mg/L and the purified proteins exhibited a simple SEC profile. The amino acid sequences of the two polypeptide chains are shown in SEQ ID NOS: 180 (second chain) and 181 (first chain).

Format 15 (F15): CD19-F15-NKp46-3

The domain structure of the trimeric F15 polypeptides is shown in FIG. 2E. The F15 polypeptide is a dimeric polypeptide which shares the structure of the F6 format, but differs by inversion of the N-terminal V_{H}-CH1 and V_{K}-C_{K} units between the central and second chains. Proteins were cloned, produced and purified as in Example 2-1. Bispecific proteins were purified from the cell culture supernatant by affinity chromatography using prot-A beads and analyzed and purified by SEC. The protein showed a good production yield of 0.9 mg/L and the purified proteins possessed a simple SEC profile. The amino acid sequences of the three polypeptide chains are shown in SEQ ID NOS: 182 (second chain), 183 (first chain) and 184 (third chain).

Format 16 (F16): CD19-F16-NKp46-3

The domain structure of the trimeric F16 polypeptide is shown in FIG. 2E. The F16 polypeptide is a dimeric polypeptide which shares the structure of the F6 format, but differs by inversion of the C-terminal V_{H}-CK and V_{K}-CH1 units between the central and second chains. Proteins were cloned, produced and purified as in Example 2-1. The amino acid sequences of the three polypeptide chains are shown in SEQ ID NOS: 185 (second chain), 186 (first chain) and 187 (third chain).

Format T5 (T5)

The domain structure of a trimeric T5 polypeptide is shown in FIG. 2F. The T5 polypeptide is a trimeric polypeptide which shares the structure of the F5 format, but differs by fusion of an scFv unit at the C-terminus of the third chain (the chain lacking the Fc domain). This protein will therefore have two antigen binding domains for antigens of interest, and one for NKp46, and will bind CD16 via its Fc domain. Proteins were cloned, produced and purified as in Example 2-1. The T5 protein had two antigen binding domains that bind human CD20, originating from different antibodies (and binding to different epitopes on CD20). The first anti-CD20 ABD contained the VH and VL of the parent antibody GA101 (GAZYVA®, Gazyvaro®, obinutuzumab, Roche Pharmaceuticals). The second anti-CD20 ABD contained the V_{H} and V_{L} of the parent antibody rituximab (Rituxan®, Mabthera®, Roche Pharmaceuticals). The third antigen binding domain binds human NKp46. The amino acid sequences of the three chains of the T5 protein are shown below (Rituximab sequences are in bold and underlined, anti-GA101 sequences are underlined, anti-NKp46 sequences are in italics).

GA101-T5-Ritux-NKp46
Polypeptide 1
(SEQ ID NO: 188)
QVQLVQSGAEVKKPGSSVKVSCKASGYAFSYSWINWVRQAPGQGLEWMGR
IFPGDGDTDYNGKFKGRVTITADKSTSTAYMELSSLRSEDTAVYYCARNV
FDGYWLVYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGST
GSQVQLQQPGAELVKPGASVKMSCKASGYTFTSYNMHWVKQTPGRGLEWI
GAIYPGNGDTSYNQKFKGKATLTADKSSSTAYMQLSSLTSEDSAVYYCAR
STYYGGDWYFNVWGAGTTVTVSARTVAAPSVFIFPPSDEQLKSGTASVVC
LLNNFYPREAKVQWKVDNALQSGNSQESVTEQDSKDSTYSLSSTLTLSKA
DYEKHKVYACEVTHQGLSSPVTKSFNRGEC-
Polypeptide 2
(SEQ ID NO: 189)
DIVMTQTPLSLPVTPGEPASISCRSSKSLLHSNGITYLYWYLQKPGQSPQ
LLIYQMSNLVSGVPDRFSGSGSGTDFTLKISRVEAEDVGVYYCAQNLELP
YTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGECDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK-
Polypeptide 3:
(SEQ ID NO: 190)
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIKASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKV
DKRVEPKSCDKTHGGSSSEVQLQQSGPELVKPGASVKISCKTSGYTFTEY
TMHWVKQSHGKSLEWIGGISPNIGGTSYNQKFKGKATLTVDKSSSTAYME
LRSLTSEDSAVYYCARRGGSFDYWGQGTTLTVSSVEGGSGGSGGSGGSGG
VDDIVMTQSPATLSVTPGDRVSLSCRASQSISDYLHWYQQKSHESPRLLI
KYASQSISGIPSRFSGSGSGSDFTLSINSVEPEDVGVYYCQNGHSFPLTF
GAGTKLELK-

Format T6 (T6)

The domain structure of the trimeric T6 polypeptide is shown in FIG. 2F. The T6 polypeptide is a trimeric polypeptide which shares the structure of the F6 format, but differs by the fusion of an scFv unit at the C-terminus of the third chain (the chain lacking the Fc domain). This trimeric protein contains two antigen binding domains for antigens of interest, and one for NKp46, and does not bind CD16 via its Fc domain due to the N297 substitution. Proteins were cloned, produced and purified as in Example 2-1. The T6 protein contains two antigen binding domains that bind human CD20. The first anti-CD20 ABD comprises the V_{H} and V_{L} of the parent antibody GA101 and the second anti-CD20 ABD comprises the V_{H} and V_{L} of rituximab. The amino acid sequences of the three chains of the T6 proteins are shown in SEQ ID NOS: 191, 192 and 193.

Format T9B (T9B)

The domain structure of the trimeric T9B polypeptide is shown in FIG. 2F. The T9B polypeptide is a trimeric polypeptide which shares the structure of the F9 format (F9B variant), but differs by the fusion of an scFv unit at the C-terminus of the free CH1 domain (on the third chain). This protein contains two antigen binding domains for antigen of interest, and one for NKp46, but will not bind CD16 via its Fc domain due to the monomeric Fc domain and/or the N297 substitution. Trimeric proteins as above described were cloned, produced and purified as in Example 2-1. The T9B protein had two antigen binding domains that bind human CD20. The first anti-CD20 ABD contained the V_{H} and V_{L} of the parent antibody GA101 and the second anti-CD20 ABD contained the V_{H} and V_{L} of the parent antibody rituximab. The amino acid sequences of the three chains of the T9B proteins are shown below.

### GA101-T9B-Ritux-NKp46

Polypeptide 2: (SEQ ID NO: 195)
DIVMTQTPLSLPVTPGEPASISCRSSKSLLHSNGITYLYWYLQKPGQSPQ
LLIYQMSNLVSGVPDRFSGSGSGTDFTLKISRVEAEDVGVYYCAQNLELP
YTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC-
Polypeptide 1: (SEQ ID NO: 194)
QVQLVQSGAEVKKPGSSVKVSCKASGYAFSYSWINWVRQAPGQGLEWMGR
IFPGDGDTDYNGKFKGRVTITADKSTSTAYMELSSLRSEDTAVYYCARNV
FDGYWLVYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTSPPSPAPELLGGPSVFLFPPKPK
DTLMISRTPEVTCVVVDVSHEDPEVKFNWYVDGVEVHNAKTKPREEQYSS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGGG
GGSGGGGSGGGGSGQPREPQVYTLPPSREEMTKNQVSLTCLVKGFYPSDI
AVEWESNGQPENNYKTTPPVLDSDGSFFLYSKLTVDKSRWQQGNVFSCSV
MHEALHNHYTQKSLSLSPGSTGSQVQLQQPGAELVKPGASVKMSCKASGY
TFTSYNMHWVKQTPGRGLEWIGAIYPGNGDTSYNQKFKGKATLTADKSSS
TAYMQLSSLTSEDSAVYYCARSTYYGGDWYFNVWGAGTTVTVSARTVAAP
SVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGE
C-
Polypeptide 3 (SEQ ID NO: 196):
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIKASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKV
DKRVEPKSCDKTHGGSSSEVQLQQSGPELVKPGASVKISCKTSGYTFTEY
TMHWVKQSHGKSLEWIGGISPNIGGTSYNQKFKGKATLTVDKSSSTAYME
LRSLTSEDSAVYYCARRGGSFDYWGQGTTLTVSSVEGGSGGSGGSGGSGG
VDDIVMTQSPATLSVTPGDRVSLSCRASQSISDYLHWYQQKSHESPRLLI
KYASQSISGIPSRFSGSGSGSDFTLSINSVEPEDVGVYYCQNGHSFPLTF
GAGTKLELK-

Format T11 (T1): CD19-T11-NKp46-3

The domain structure of the dimeric T11 polypeptide is shown in FIG. 2F. The T11 polypeptide is a trimeric polypeptide which shares the structure of the F11 format, but differs by the fusion of an scFv unit at the C-terminus of the free CH1 domain. This dimeric protein contains two antigen binding domains for antigen of interest, and one for NKp46, and does not bind CD16 via its Fc domain due to the monomeric Fc domain and/or the N297 substitution. Proteins were cloned, produced and purified as in Example 2-1. The T11 protein contains two antigen binding domains that bind human CD20. The first anti-CD20 ABD contained the VH and VL of the parent antibody GA101 and the second anti-CD20 ABD contained the VH and VL of rituximab. The amino acid sequences of the two chains of the T11 protein are shown below.

### GA101-T11-Ritux-NKp46

Polypeptide 1 (SEQ ID NO: 197):
DIVMTQTPLSLPVTPGEPASISCRSSKSLLHSNGITYLYWYLQKPGQSPQ
LLIYQMSNLVSGVPDRFSGSGSGTDFTLKISRVEAEDVGVYYCAQNLELP
YTFGGGTKVEIKGGGGSGGGGSGGGGSQVQLVQSGAEVKKPGSSVKVSCK
ASGYAFSYSWINWVRQAPGQGLEWMGRIFPGDGDTDYNGKFKGRVTITAD
KSTSTAYMELSSLRSEDTAVYYCARNVFDGYWLVYWGQGTLVTVSSASTK
GPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCD
KTHTSPPSPAPELLGGPSVFLFPPKPKDTLMISRTPEVTCVVVDVSHEDP
EVKFNWYVDGVEVHNAKTKPREEQYSSTYRVVSVLTVLHQDWLNGKEYKC
KVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGN
VFSCSVMHEALHNHYTQKSLSLSPGGGGGSGGGGSGGGGSGQPREPQVYT
LPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGSTGS
QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSARTVAAPSVFIFPPSDEQLKSGTASVVCLL
NNFYPREAKVQWKVDNALQSGNSQESVTEQDSKDSTYSLSSTLTLSKADY
EKHKVYACEVTHQGLSSPVTKSFNRGEC-
Polypeptide 2 (SEQ ID NO: 198):
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIKASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKV
DKRVEPKSCDKTHGGSSSEVQLQQSGPELVKPGASVKISCKTSGYTFTEY
TMHWVKQSHGKSLEWIGGISPNIGGTSYNQKFKGKATLTVDKSSSTAYME
LRSLTSEDSAVYYCARRGGSFDYWGQGTTLTVSSVEGGSGGSGGSGGSGG
VDDIVMTQSPATLSVTPGDRVSLSCRASQSISDYLHWYQQKSHESPRLLI
KYASQSISGIPSRFSGSGSGSDFTLSINSVEPEDVGVYYCQNGHSFPLTF
GAGTKLELK-

### Example 5

### NKp46 Binding Affinity by Bispecific Proteins by Surface Plasmon Resonance (SPR)

### Biacore T100 General Procedure and Reagents

SPR measurements were performed on a Biacore T100 apparatus (Biacore GE Healthcare) at 25° C. In all Biacore experiments HBS-EP+ (Biacore GE Healthcare) and NaOH 10 mM served as running buffer and regeneration buffer respectively. Sensorgrams were analyzed with Biacore T100 Evaluation software. Protein-A was purchased from (GE Healthcare). Human NKp46 recombinant proteins were cloned, produced and purified at Innate Pharma.

### Immobilization of Protein-A

Protein-A proteins were immobilized covalently to carboxyl groups in the dextran layer on a Sensor Chip CM5. The chip surface was activated with EDC/NHS (N-ethyl-N′-(3-dimethylaminopropyl) carbodiimidehydrochloride and N-hydroxysuccinimide (Biacore GE Healthcare)). Protein-A was diluted to 10 μg/ml in coupling buffer (10 mM acetate, pH 5.6) and injected until the appropriate immobilization level was reached (i.e. 2000 RU). Deactivation of the remaining activated groups was performed using 100 mM ethanolamine pH 8 (Biacore GE Healthcare).

### Binding Study

The bispecific proteins were first tested in Format F1 described in Example 2 having different anti-NKp46 variable regions from NKp46-1, NKp46-2, NKp46-3 or NKp46-4 antibodies. Antibodies were next tested as different formats F3, F4, F5, F6, F9, F10, F11, F13, F14 having the anti-NKp46 variable regions from the NKp46-3 antibody, and compared to the NKp46-3 antibody as a full-length human IgG1.

Bispecific proteins at 1 μg/mL were captured onto Protein-A chip and recombinant human NKp46 proteins were injected at 5 μg/mL over captured bispecific antibodies. For blank subtraction, cycles were performed again replacing NKp46 proteins with running buffer.

The Bab281 antibody was separately tested for binding to NKp46 by SPR, and additionally by flow cytometry using a cell line expressing the human NKp46 construct at the cell surface. For FACS screening, the presence of reacting antibodies in supernanants was revealed by Goat anti-mouse polyclonal antibody (pAb) labeled with PE. SPC and FACS results showed that the Bab281 based antibody did not bind the NKp46 cell line or NKp46-Fc proteins. Bab281 lost binding to its target when presented in the bispecific format.

### Affinity Study

Monovalent affinity study was done following a regular Capture-Kinetic protocol recommended by the manufacturer (Biacore GE Healthcare kinetic wizard). Seven serial dilutions of human NKp46 recombinant proteins, ranging from 6.25 to 400 nM were sequentially injected over the captured Bi-Specific antibodies and allowed to dissociate for 10 min before regeneration. The entire sensorgram sets were fitted using the 1:1 kinetic binding model.

### Results

SPR showed that the bispecific polypeptides of format F1 having the NKp46-1, 2, 3 and 4 scFv binding domains bound to NKp46, while other bispecific polypeptides having the scFv of other anti-NK46 antibodies did not retain NKp46 binding. The binding domains that did not retain binding in monomeric bispecific format initially bound to NKp46 but lost binding upon conversion to the bispecific format. All of the bispecific polypeptides of formats F1, F2 F3, F4, F5, F6, F9, F10, F11, F13, F14 retained binding to NKp46 when using the NKp46-3 variable regions. Monovalent affinities and kinetic association and dissociation rate constants are shown below in the Table 3 below.

TABLE 3

| Bispecific mAb | ka (1/Ms) | kd (1/s) | KD (M) |
|---|---|---|---|
| CD19-F1-Bab281 | n/a | n/a | n/a |
| | | | (loss of binding) |
| CD19-F1-NKp46-1 | 1.23E+05 | 0.001337 | 1.09E−08 |
| CD19-F1-NKp46-2 | 1.62E+05 | 0.001445 | 8.93E−09 |
| CD19-F1-NKp46-3 | 7.05E+04 | 6.44E−04 | 9.14E−09 |
| CD19-F1-NKp46-4 | 1.35E+05 | 6.53E−04 | 4.85E−09 |
| CD19-F3-NKp46-3 | 3.905E+5 | 0.01117 | 28E−09 |
| CD19-F4-NKp46-3 | 3.678E+5 | 0.01100 | 30E−09 |
| CD19-F5-NKp46-3 | 7.555E+4 | 0.00510 | 67E−09 |
| CD19-F6-NKp46-3 | 7.934E+4 | 0.00503 | 63E−09 |
| CD19-F9A-NKp46-3 | 2.070E+5 | 0.00669 | 32E−09 |
| CD19-F10A-NKp46-3 | 2.607E+5 | 0.00754 | 29E−09 |
| CD19-F11A-NKp46-3 | 3.388E+5 | 0.01044 | 30E−09 |
| CD19-F13-NKp46-3 | 8.300E+4 | 0.00565 | 68E−09 |
| CD19-F14-NKp46-3 | 8.826E+4 | 0.00546 | 62E−09 |
| NKp46-3 IgG1 | 2.224E+5 | 0.00433 | 20E−09 |

### Example 6

### Engagement of NK Cells Against Daudi Tumor Target with Fc-Containing NKp46 x CD19 Bispecific Protein

Bispecific antibodies having a monomeric Fc domain and a domain arrangement according to the single chain F1 or dimeric F2 formats described in Example 3, and a NKp46 binding region based on NKp46-1, NKp46-2, NKp46-3 or NKp46-4 were tested for functional ability to direct NK cells to lyse CD19-positive tumor target cells (Daudi, a well characterized B lymphoblast cell line). The F2 proteins additionally included NKp46-9 variable regions which lost binding to NKp46 in the scFv format but which retained binding in the F(ab)-like format of F2.

Briefly, the cytolytic activity of each of (a) resting human NK cells, and (b) human NK cell line KHYG-1 transfected with human NKp46, was assessed in a classical 4-h 51Cr-release assay in U-bottom 96 well plates. Daudi cells were labelled with 51Cr (50 μCi (1.85 MBq)/1×106 cells), then mixed with KHYG-1 transfected with hNKp46 at an effector/target ratio equal to 50 for KHYG-1, and 10 (for F1 proteins) or 8.8 (for F2 proteins) for resting NK cells, in the presence of monomeric bi-specific antibodies at different concentrations. After brief centrifugation and 4 hours of incubation at 37° C., samples of supernatant were removed and transferred into a LumaPlate (Perkin Elmer Life Sciences, Boston, MA), and 51Cr release was measured with a TopCount NXT beta detector (PerkinElmer Life Sciences, Boston, MA). All experimental conditions were analyzed in triplicate, and the percentage of specific lysis was determined as follows: 100×(mean cpm experimental release−mean cpm spontaneous release)/(mean cpm total release−mean cpm spontaneous release). Percentage of total release is obtained by lysis of target cells with 2% Triton X100 (Sigma) and spontaneous release corresponds to target cells in medium (without effectors or Abs).

Results

In the KHYG-1 hNKp46 NK experimental model, each bi-specific antibody NKp46-1, NKp46-2, NKp46-3, NKp46-4 or NKp46-9 induced specific lysis of Daudi cells by human KHYG-1 hNKp46 NK cell line compared to negative controls (Human IgG1 isotype control (IC) and CD19/CD3 bi-specific antibodies), thereby showing that these antibodies induce Daudi target cell lysis by KHYG-1 hNKp46 through CD19/NKp46 cross-linking.

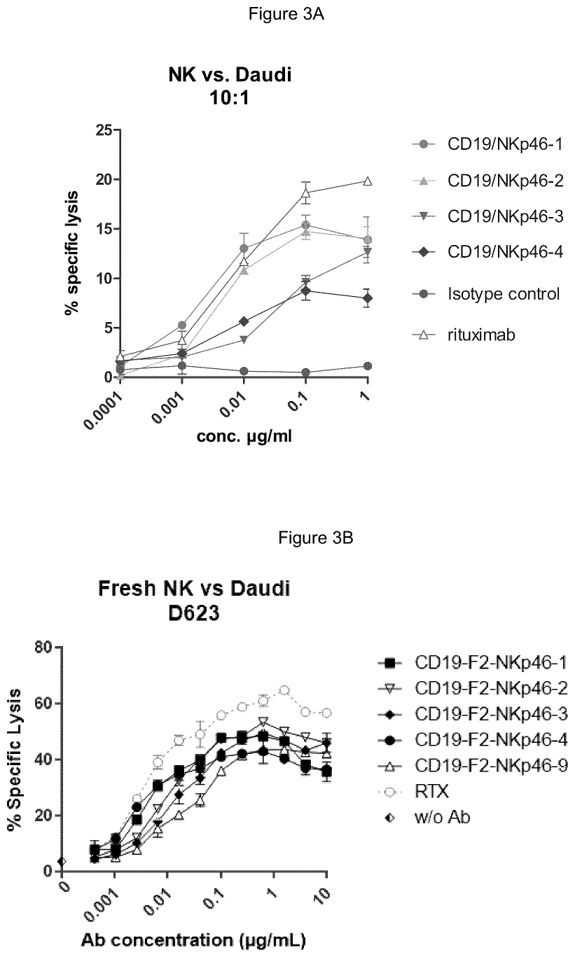

When resting NK cells were used as effectors, each bi-specific antibody NKp46-1, NKp46-2, NKp46-3, NKp46-4 or NKp46-9 again induced specific lysis of Daudi cells by human NK cells compared to negative control (Human IgG1 isotype control (IC) antibody), thereby showing that these antibodies induce Daudi target cell lysis by human NK cells through CD19/NKp46 cross-linking. Rituximab (RTX, chimeric IgG1) was used as a positive control of ADCC (Antibody-Dependent Cell Cytotoxicity) by resting human NK cells. The maximal response obtained with RTX (at 10 μg/ml in this assay) was 21.6% specific lysis illustrating that the bispecific antibodies have high target cell lysis activity. Results for experiments with resting NK cells are shown in FIG. 3A for the single chain F1 proteins and 3B for the dimeric F2 proteins.

### Example 7

### Comparison with Full Length anti-NKp46 mAbs and Depleting Anti-Tumor mAbs: Only NKp46 x CD19 Bispecific Proteins Prevent Non-Specific NK Activation

These studies aimed to investigate whether bispecific antibodies can mediate NKp46-mediated NK activation toward cancer target cells without triggering non-specific NK cell activation.

NKp46 x CD19 bispecific proteins having an arrangement according to the F2 format described in Example 3 with anti-NKp46 variable domains from NKp46-1, NKp46-2, NKp46-3, NKp46-4 or NKp46-9 were compared to:

(a) full-length monospecific anti-NKp46 antibodies (NKp46-3 as human IgG1), and

(b) the anti-CD19 antibody as a full-length human IgG1 as ADCC inducing antibody control comparator.

The experiments further included as controls: rituximab, an anti-CD20 ADCC inducing antibody control for a target antigen with high expression levels; anti-CD52 antibody alemtuzumab, a human IgG1, binds CD52 target present on both targets and NK cells; and negative control isotype control therapeutic antibody (a human IgG1 that does not bind a target present on the target cells (HUG1-IC).

The different proteins were tested for functional effect on NK cell activation in the presence of CD19-positive tumor target cells (Daudi cells), in the presence of CD19-negative, CD16-positive target cells (HUT78 T-lymphoma cells), and in the absence of target cells.

Briefly, NK activation was tested by assessing CD69 and CD107 expression on NK cells by flow cytometry. The assay was carried out in 96 U well plates in completed RPMI, 150 μL final/well. Effector cells were fresh NK cells purified from donors. Target cells were Daudi (CD19-positive), HUT78 (CD19-negative) or K562 (NK activation control cell line). In addition to K562 positive control, three conditions were tested, as follows:
>NK Cell Alone
>NK cells vs Daudi (CD19+)
>NK cells vs HUT78 (CD19−)

Effector:Target (E:T) ratio was 2.5:1 (50 000 E:20 000 T), with an antibody dilution range starting to 10 μg/mL with ¼ dilution (n=8 concentrations). Antibodies, target cells and effector cells were mixed; spun 1 min at 300 g; incubated 4 h at 37° C.; spun 3 min at 500 g; washed twice with Staining Buffer (SB); added 50 μL of staining Ab mix; incubated 30 min at 300 g; washed twice with SB resuspended pellet with CellFix ; stored overnight at 4° C.; and fluorescence revealed with Canto II (HTS).

Results

1. NK Cells Alone

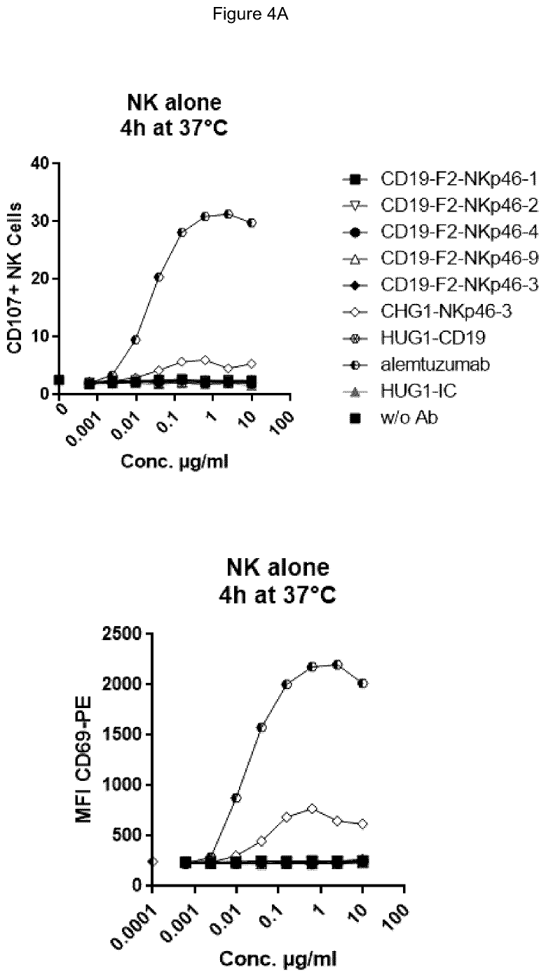

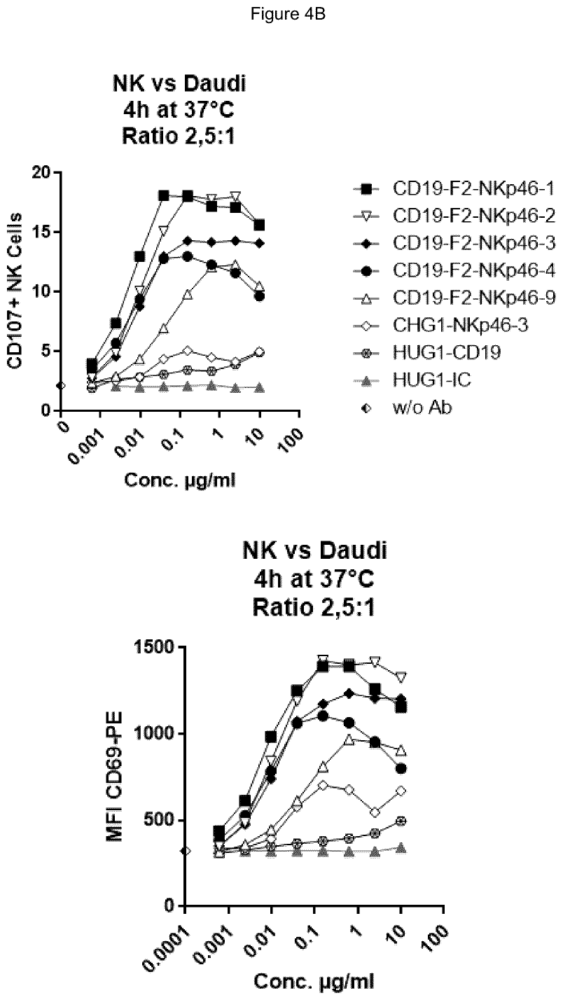

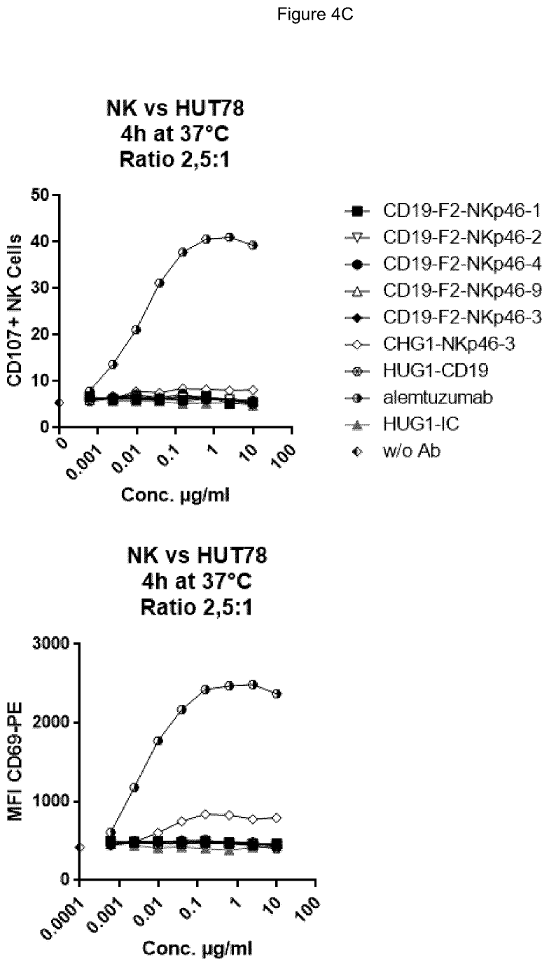

Results are shown in FIG. 4A. In the absence of target-antigen expressing cells, none of the bispecific anti-NKp46 x anti-CD19 antibody (including each of the NKp46-1, NKp46-2, NKp46-3, NKp46-4 and NKp46-9 variable regions) activated NK cells as assessed by CD69 or CD107 expression. Full-length anti-CD19 also did not activate NK cells. However, the full-length anti-NKp46 antibodies caused detectable activation of NK cells. Alemtuzumab also induced activation of NK cells, at a very high level. Isotype control antibody did not induce activation.

2. NK Cells vs Daudi (CD19+)

Results are shown in FIG. 4B. In the presence of target-antigen expressing cells, each of the bispecific anti-NKp46 x anti-CD19 antibodies (including each of the NKp46-1, NKp46-2, NKp46-3, NKp46-4 and NKp46-9 binding domains) activated NK cells. Full-length anti-CD19 showed at best only very low activation of NK cells. Neither full-length anti-NKp46 antibodies or alemtuzumab showed substantial increase in activation beyond what was observed in presence of NK cells alone. FIG. 4 shows full-length anti-NKp46 antibodies showed a similar level of baseline activation observed in presence of NK cells alone. Alemtuzumab also induced activation of NK cells a similar level of activation observed in presence of NK cells alone, and at higher antibody concentrations in this setting (ET 2.5:1) the activation was greater than with the bispecific anti-NKp46 x anti-CD19 antibody. Isotype control antibody did not induce activation.

3. NK Cells vs HUT78 (CD19−)

Results are shown in FIG. 4C. In the presence of target-antigen-negative HUT78 cells, none of the bispecific anti-NKp46 x anti-CD19 antibody (including each of the NKp46-1, NKp46-2, NKp46-3, NKp46-4 and NKp46-9 variable regions) activated NK cells. However, the full-length anti-NKp46 antibodies and alemtuzumab caused detectable activation of NK cells at a similar level observed in presence of NK cells alone. Isotype control antibody did not induce activation.

In conclusion, the bispecific anti-NKp46 proteins are able to activate NK cells in a target-cell specific manner, unlike full-length monospecific anti-NKp46 antibodies and full-length antibodies of depleting IgG isotypes which also activate NK cells in the absence of target cells. The NK cell activation achieved with anti-NKp46 bispecific proteins was higher than that observed with full length anti-CD19 IgG1 antibodies.

### Example 8

### Comparative Efficacy with Depleting Anti-Tumor mAbs: NKp46 x CD19 Bispecific Proteins at Low ET Ratio

These studies aimed to investigate whether bispecific antibodies can mediate NKp46-mediated NK cell activation toward cancer target cells at lower effector:target ratios. The ET ratio used in this Example was 1:1 which is believed to be closer to the setting that would be encountered in vivo than the 2.5:1 ET ratio used in Example 7 or the 10:1 ET ratio of Example 6.

NKp46 x CD19 bispecific proteins having an arrangement according to the F2 format described in Example 3 with anti-NKp46 variable domains from NKp46-1, NKp46-2, NKp46-3, NKp46-4 or NKp46-9 were compared to:

(a) full-length monospecific anti-NKp46 antibodies (NKp46-3 as human IgG1), and

(b) the anti-CD19 antibody as a full-length human IgG1 as ADCC inducing antibody control comparator.

The experiments further included as controls: rituximab (an anti-CD20 ADCC inducing antibody control for a target antigen with high expression levels); anti-CD52 antibody alemtuzumab (a human IgG1, binds CD52 target present on both targets and NK cells), and negative control isotype control therapeutic antibody (a human IgG1 that does not bind a target present on the target cells (HUG1-IC). The different proteins were tested for functional effect on NK cell activation as assessed by CD69 or CD107 expression in the presence of CD19-positive tumor target cells (Daudi cells), in the presence of CD19-negative, CD16-positive target cells (HUT78 T-lymphoma cells), and in the absence of target cells. The experiments were carried out as in Example 7 except that the ET ratio was 1:1.

Results

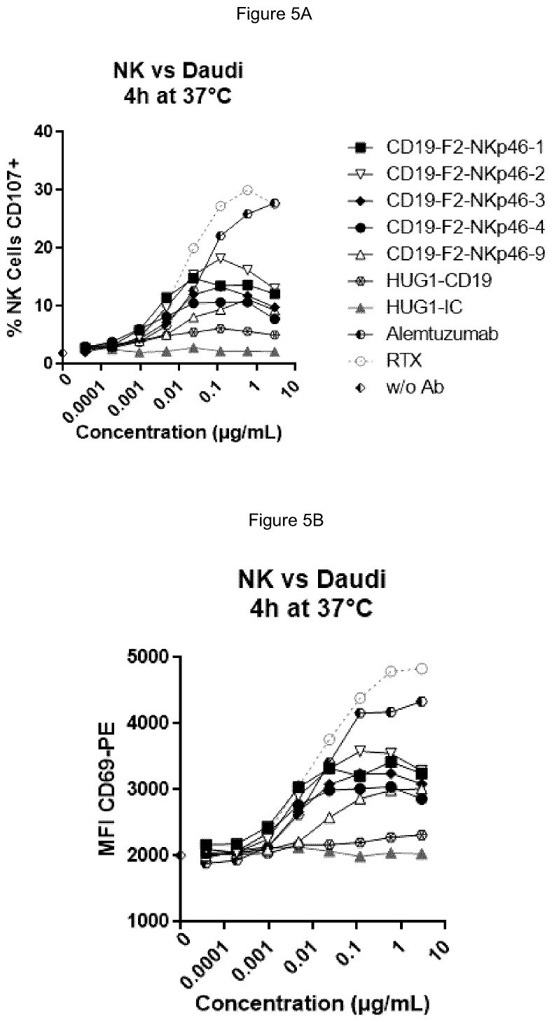

Results are shown in FIG. 5 (5A: CD107 and 5B: CD69). In the presence of target-antigen expressing cells, each of the bispecific anti-NKp46 x anti-CD19 antibody (including each of the NKp46-1, NKp46-2, NKp46-3, NKp46-4 and NKp46-9 variable regions) activated NK cells in the presence of Daudi cells.

The activation induced by bispecific anti-NKp46 x anti-CD19 antibody in the presence of Daudi cells was far more potent than the full-length human IgG1 anti-CD19 antibody as ADCC inducing antibody which had low activity in this setting. Furthermore, in this low E:T ratio setting the activation induced by bispecific anti-NKp46 x anti-CD19 antibody was as potent as anti-CD20 antibody rituximab, with a difference being observed only at the highest concentrations that were 10 fold higher than concentrations in which differences were observed at the 2.5:1 ET ratio.

In the absence of target cells or in the presence of target antigen-negative HUT78 cells, full-length anti-NKp46 antibodies and alemtuzumab showed a similar level of baseline activation observed in the presence of Daudi cells. Anti-NKp46 x anti-CD19 antibody did not activate NK cells in presence of HUT78 cells.

In conclusion, the bispecific anti-NKp46 proteins are able to activate NK cells in a target-cell specific manner and at lower effector:target ratio are more effective in mediating NK cell activation that traditional human IgG1 antibodies.

### Example 9

### NKp46 Mechanism of Action

NKp46 x CD19 bispecific proteins having an arrangement according to the F2, F3, F5 or F6 formats described in Examples 3 or 4 with anti-NKp46 variable domains from NKp46-3 were compared to rituximab (anti-CD20 ADCC inducing antibody), and a human IgG1 isotype control antibody for functional ability to direct CD16−/NKp46+ NK cell lines to lyse CD19-positive tumor target cells.

Briefly, the cytolytic activity of the CD16−/NKp46+ human NK cell line KHYG-1 was assessed in a classical 4-h ⁵¹Cr-release assay in U-bottom 96 well plates. Daudi or B221 cells were labelled with ⁵¹Cr (50 μCi (1.85 MBq)/1×10⁶ cells), then mixed with KHYG-1 at an effector/target ratio equal to 50:1, in the presence of test antibodies at dilution range starting from 10⁻⁷ mol/L with ⅕ dilution (n=8 concentrations).

After brief centrifugation and 4 hours of incubation at 37° C., 50 μL of supernatant were removed and transferred into a LumaPlate (Perkin Elmer Life Sciences, Boston, MA), and ⁵¹Cr release was measured with a TopCount NXT beta detector (PerkinElmer Life Sciences, Boston, MA). All experimental conditions were analyzed in triplicate, and the percentage of specific lysis was determined as follows: 100×(mean cpm experimental release−mean cpm spontaneous release)/(mean cpm total release−mean cpm spontaneous release). Percentage of total release is obtained by lysis of target cells with 2% Triton X100 (Sigma) and spontaneous release corresponds to target cells in medium (without effectors or Abs).

Results

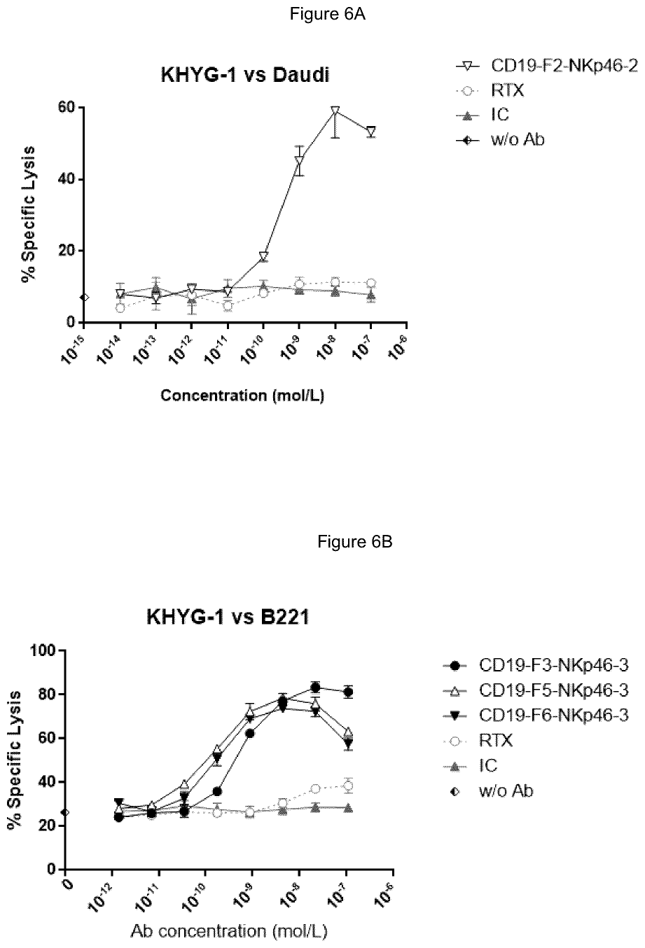

Results are shown in FIG. 6A (KHYG-1 vs Daudi) and FIG. 6B (KHYG-1 vs B221). In the KHYG-1 hNKp46 NK experimental model, each NKp46 x CD19 bispecific protein (Format F2, F3, F5 and F6) induced specific lysis of Daudi or B221 cells by human KHYG-1 hNKp46 NK cell line, while rituximab and human IgG1 isotype control (IC) antibodies did not.

### Example 10

### Comparative Efficacy of Bispecific and Conventional IgG Antibody Using Fresh Human NK Cells

NKp46 x CD19 bispecific proteins that bind human CD16 having an arrangement according to the F5 format with anti-NKp46 variable domains from NKp46-3 were compared to the same bispecific antibody as a F6 format (which lacks CD16 binding), and to a human IgG1 isotype anti-CD19 antibody, as well as a human IgG1 isotype control antibody for functional ability to direct purified NK cells to lyse CD19-positive Daudi tumor target cells.

Briefly, the cytolytic activity of fresh human purified NK cells from EFS Buffy Coat was assessed in a classical 4-h ⁵¹Cr-release assay in U-bottom 96 well plates. Daudi or HUT78 cells (negative control cells that do not express CD19) were labelled with ⁵¹Cr and then mixed with NK cells at an effector/target ratio equal to 10:1, in the presence of test antibodies at dilution range starting from 10 g/ml with 1/10 dilution (n=8 concentrations).

After brief centrifugation and 4 hours of incubation at 37° C., 50 μL of supernatant were removed and transferred into a LumaPlate (Perkin Elmer Life Sciences, Boston, MA), and ⁵¹Cr release was measured with a TopCount NXT beta detector (PerkinElmer Life Sciences, Boston, MA). All experimental conditions were analyzed in triplicate, and the percentage of specific lysis was determined as follows: 100×(mean cpm experimental release−mean cpm spontaneous release)/(mean cpm total release−mean cpm spontaneous release). Percentage of total release is obtained by lysis of target cells with 2% Triton X100 (Sigma) and spontaneous release corresponds to target cells in medium (without effectors or Abs).

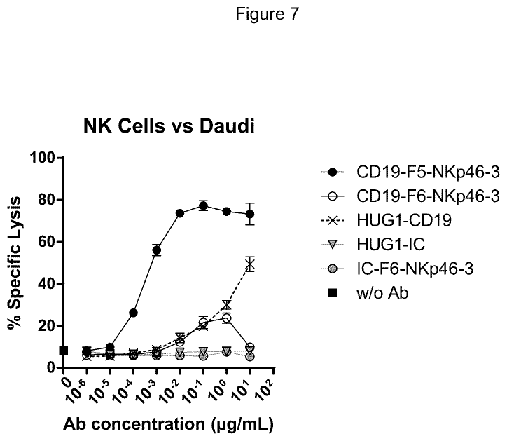

Results are shown in FIG. 7. The CD19-F6-NKp46 (bispecific protein in F6 format) whose Fc domain does not bind CD16 due to a N297 substitution was as potent in mediating NK cell lysis of Daudi target cells as the full-length IgG1 anti-CD19 antibody, which is remarkable considering that the control IgG1 anti-CD19 antibody binds CD19 bivalently and that the anti-CD19 is bound by CD16. Surprisingly, the CD19-F6-NKp46 (F5 format protein) whose Fc domain additionally binds CD16 is far more potent in mediating Daudi target cell lysis that the full-length IgG1 anti-CD19 antibody or the F6 format bispecific protein. At comparable levels of target cell lysis, the CD19-F6-NKp46 was at least 1000 times more potent than the full-length anti-CD19 IgG1.

### Example 11

### Binding of Different Bispecific Formats to FcRn

Affinity of different antibody formats for human FcRn was studied by Surface Plasmon Resonance (SPR) by immobilizing recombinant FcRn proteins covalently to carboxyl groups in the dextran layer on a Sensor Chip CM5, as described in Example 2-6.

A chimeric full length anti-CD19 antibody having human IgG1 constant regions and NKp46 x CD19 bispecific proteins having an arrangement according to the F3, F4, F5, F6, F9, F10, F11, F13 or F14 formats described in Examples 3 or 4 with anti-NKp46 variable domains from NKp46-3 (NKp46-2 for F2) were tested; for each analyte, the entire sensorgram was fitted using the steady state or 1:1 SCK binding model. Results are shown in Table 4 below. The bispecific proteins having dimeric Fc domains (formats F5, F6, F13, F14) bound to FcRn with affinity similar to that of the full-length IgG1 antibody. The bispecific proteins with monomeric Fc domains (F3, F4, F9, F10,

F11) also displayed binding to FcRn, however with lower affinity that the bispecific proteins having dimeric Fc domains.

TABLE 4

| Antibody/Bispecific | SPR method | KD nM |
|---|---|---|
| Human IgG1/K Anti- | SCK/Two state | 7.8 |
| CD19 | reaction | |
| CD19-F5-NKp46-3 | SCK/Two state | 2.6 |
| | reaction | |
| CD19-F6-NKp46-3 | SCK/Two state | 6.0 |
| | reaction | |
| CD19-F13-NKp46-3 | SCK/Two state | 15.2 |
| | reaction | |
| CD19-F14-NKp46-3 | SCK/Two state | 14.0 |
| | reaction | |
| CD19-F3-NKp46-3 | Steady State | 474.4 |
| CD19-F4-NKp46-3 | Steady State | 711.7 |
| CD19-F9A-NKp46-3 | Steady State | 858.5 |
| CD19-F10A-NKp46-3 | Steady State | 432.8 |
| CD19-F11-NKp46-3 | Steady State | 595.5 |

### Example 12

### Binding to FcγR

Different multimeric Fc proteins were evaluated to assess whether such bispecific monomeric Fc protein could retain binding to Fcγ receptors.

SPR measurements were performed on a Biacore T100 apparatus (Biacore GE Healthcare) at 25° C. In all Biacore experiments HBS-EP+ (Biacore GE Healthcare) and 10 mM NaOH, 500 mM NaCl served as running buffer and regeneration buffer respectively. Sensorgrams were analyzed with Biacore T100 Evaluation software. Recombinant human FcRs (CD64, CD32a, CD32b, CD16a and CD16b) were cloned, produced and purified.

F5 and F6 bispecific antibodies CD19-F5-NKp46-3 or CD19-F6-NKp46-3 were immobilized covalently to carboxyl groups in the dextran layer on a Sensor Chip CM5. The chip surface was activated with EDC/NHS (N-ethyl-N′-(3-dimethylaminopropyl) carbodiimidehydrochloride and N-hydroxysuccinimide (Biacore GE Healthcare)). Bispecific antibodies were diluted to 10 μg/ml in coupling buffer (10 mM acetate, pH 5.6) and injected until the appropriate immobilization level was reached (i.e. 800 to 900 RU). Deactivation of the remaining activated groups was performed using 100 mM ethanolamine pH 8 (Biacore GE Healthcare).

Monovalent affinity study was assessed following a classical kinetic wizard (as recommended by the manufacturer). Serial dilutions of soluble analytes (FcRs) ranging from 0.7 to 60 nM for CD64 and from 60 to 5000 nM for all the other FcRs were injected over the immobilized bispecific antibodies and allowed to dissociate for 10 min before regeneration. The entire sensorgram sets were fitted using the 1:1 kinetic binding model for CD64 and with the Steady State Affinity model for all the other FcRs.

While full length wild type human IgG1 bound to all cynomolgus and human Fcy receptors, the CD19-F6-NKp46-3 bi-specific antibodies did not bind to any of the receptors. The CD19-F5-NKp46-3, on the other hand, bound to each of the human receptors CD64 (KD=0.7 nM), CD32a (KD=846 nM), CD32b (KD=1850 nM), CD16a (KD=1098 nM) and CD16b (KD-2426 nM). Convention human anti-IgG1 antibodies has comparable binding to the Fc receptors (KD shown in the table below).

| | | Full length human |
|---|---|---|
| | CD19-F5-NKp46-3 | IgG1 antibody |
| Human Fcγ receptor | KD (nM) | KD (nM) |
| CD64 | 0.7 | 0.24 |
| CD32a | 846 | 379 |
| CD32b | 1850 | 1180 |
| CD16a | 1098 | 630 |
| CD16b | 2426 | 2410 |

### Example 13

### Epitope Mapping of Anti-NKp46 Antibodies

A. Competition Assays

Competition assays were conducted by Surface Plasmon Resonance (SPR according to the methods described below.

SPR measurements were performed on a Biacore T100 apparatus (Biacore GE Healthcare) at 25° C. In all Biacore experiments HBS-EP+ (Biacore GE Healthcare) and NaOH 10mM NaCl 500 mM served as running buffer and regeneration buffer respectively.

Sensorgrams were analyzed with Biacore T100 Evaluation software. Anti-6xHis tag antibody was purchased from QIAGEN. Human 6xHis tagged NKp46 recombinant proteins (NKp46-His) were cloned, produced and purified at Innate Pharma.

Anti-His antibodies were immobilized covalently to carboxyl groups in the dextran layer on a Sensor Chip CM5. The chip surface was activated with EDC/NHS (N-ethyl-N′-(3-dimethylaminopropyl) carbodiimidehydrochloride and N-hydroxysuccinimide (Biacore GE Healthcare)). Protein-A and Anti-His antibodies were diluted to 10 μg/ml in coupling buffer (10 mM acetate, pH 5.6) and injected until the appropriate immobilization level was reached (i.e. 2000 to 2500 RU). Deactivation of the remaining activated groups was performed using 100 mM ethanolamine pH 8 (Biacore GE Healthcare).

Parental regular human IgG1 chimeric antibodies having NKp46 binding region corresponding to NKp46-1, NKp46-2, NKp46-3 or NKp46-4 were used for the competition study which has been performed using an Anti-6xHis tag antibody chip.

Bispecific antibodies having NKp46 binding region based on NKp46-1, NKp46-2, NKp46-3 or NKp46-4 at 1 μg/mL were captured onto Protein-A chip and recombinant human NKp46 proteins were injected at 5 μg/mL together with a second test bispecific antibody of the NKp46-1, NKp46-2, NKp46-3 or NKp46-4 group.

None of NKp46-1, NKp46-2, NKp46-3 or NKp46-4 competed with one another for binding to NKp46, these antibodies each representing a different epitope.

B. Binding to NKp46 Mutants

In order to define the epitopes of anti NKp46 antibodies, we designed NKp46 mutants defined by one, two or three substitutions of amino acids exposed at the molecular surface over the 2 domains of NKp46. This approach led to the generation of 42 mutants transfected in Hek-293T cells, as shown in the table below. The targeted amino acid mutations in the table 5 below are shown both using numbering of SEQ ID NO: 1 (also corresponding to the numbering used in Jaron-Mendelson et al. (2012) J. Immunol. 88(12):6165-74.

TABLE 5

| | Substitution | | |
|---|---|---|---|
| | (Numbering according to: Jaron- | | |
| Mutant | Mendelson and SEQ ID NO 1) | | |
| 1 | P40A | K43S | Q44A |
| 2 | K41S | E42A | E119A |
| 3 | P86A | D87A | |
| 4 | N89A | R91A | |
| 5 | K80A | K82A | |
| 5bis | E34A | T46A | |
| 6 | R101A | V102A | |
| 7 | N52A | Y53A | |
| 8 | V56A | P75A | E76A |
| 9 | R77A | I78A | |
| 10 | S97A | I99A | |
| 10bis | Q59A | H61A | |
| 11 | L66A | V69A | |
| 12 | E108A | | |
| 13 | N111A | L112A | |
| 14 | D114A | | |
| 15 | T125A | R145S | D147A |
| 16 | S127A | Y143A | |
| 17 | H129A | K139A | |
| 18 | K170A | V172A | |
| 19 | I135A | S136A | |
| 19bis | T182A | R185A | |
| 20 | R160A | | |
| 21 | K207A | | |
| 22 | M152A | R166A | |
| 23 | N195A | N196A | |
| Stalk1 | D213A | I214A | T217A |
| Stalk2 | F226A | T233A | |
| Stalk3 | L236A | T240A | |
| Supp1 | F30A | W32A | |
| Supp2 | F62A | F67A | |
| Supp3 | E63A | Q95A | |
| Supp4 | R71A | K73A | |
| Supp5 | Y84A | | |
| Supp6 | E104A | L105A | |
| Supp7 | Y121A | Y194A | |
| Supp8 | P132A | E133A | |
| Supp9 | S151A | Y168A | |
| Supp10 | S162A | H163A | |
| Supp11 | E174A | P176A | |
| Supp12 | P179A | H184A | |
| Supp13 | R189A | E204A | P205A |

### Generation of Mutants

NKp46 mutants were generated by PCR. The sequences amplified were run on agarose gel and purified using the Macherey Nagel PCR Clean-Up Gel Extraction kit (reference 740609). The two or three purified PCR products generated for each mutant were then ligated into an expression vector, with the ClonTech InFusion system. The vectors containing the mutated sequences were prepared as Miniprep and sequenced. After sequencing, the vectors containing the mutated sequences were prepared as Midiprep using the Promega PureYield™ Plasmid Midiprep System. HEK293T cells were grown in DMEM medium (Invitrogen), transfected with vectors using Invitrogen's Lipofectamine 2000 and incubated at 37° C. in a CO2 incubator for 24 hours prior to testing for transgene expression.

Flow cytometry analysis of anti-NKp46 binding to the HEK293T transfected cells

All the anti-NKp46 antibodies were tested for their binding to each mutant by flow cytometry. A first experiment was performed to determine antibodies that lose their binding to one or several mutants at one concentration (10 μg/ml). To confirm a loss of binding, titration of antibodies was done on antibodies for which binding seemed to be affected by the NKp46 mutations (1-0,1-0,01-0,001 μg/ml).

Results

Antibody NKp46-1 had decreased binding to the mutant 2 (having a mutation at residues K41, E42 and E119 (numbering in NKp46 wild-type) compared to wild-type NK46. Similarly, NKp46-1 also had decreased binding to the supplementary mutant Supp7 (having a mutation at residues Y121 and Y194.

Antibody NKp46-3 had decreased binding to the mutant 19 (having a mutation at residues 1135, and S136. Similarly, NKp46-1 also had decreased binding to the supplementary mutant Supp8 (having a mutation at residues P132 and E133.

Antibody NKp46-4 had decreased binding to the mutant 6 (having a mutation at residues R101, and V102. Similarly, NKp46-1 also had decreased binding to the supplementary mutant Supp6 having a mutation at residues E104 and L105.

In this study, we identified epitopes for anti-NKp46 antibodies (NKp46-1, NKp46-3 and NKp46-4). Epitopes of NKp46-4, NKp46-3 and NKp46-1 are on NKp46 D1 domain, D2 domain and D1/D2 junction, respectively. R101, V102, E104 and L105 are essential residues for NKp46-4 binding and defined a part of NKp46-4 epitope. The epitope of NKp46-1 epitope includes K41, E42, E119, Y121 and Y194 residues. The epitope of NKp46-3 includes P132, E133, 1135, and S136 residues.

All headings and sub-headings are used herein for convenience only and should not be construed as limiting the invention in any way. Any combination of the above-described elements in all possible variations thereof is encompassed by the invention unless otherwise indicated herein or otherwise clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate). All methods described herein can be performed in any suitable order unless otherwise indicated herein or otherwise clearly contradicted by context.

The use of any and all examples, or exemplary language (e.g., “such as”) provided herein is intended merely to better illuminate the invention and does not pose a limitation on the scope of the invention unless otherwise indicated. No language in the specification should be construed as indicating any element is essential to the practice of the invention unless as much is explicitly stated.

The description herein of any aspect or embodiment of the invention using terms such as reference to an element or elements is intended to provide support for a similar aspect or embodiment of the invention that “consists of,” “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

We claim:

1. A method of treating a disease in a subject comprising administering to the subject a composition comprising protein or polypeptide comprising an antigen binding domain that binds a human NKp46 polypeptide, wherein the domain comprises a heavy chain variable region (VH) and a light chain variable region (VL) combination selected from the group consisting of:
(a) a VH comprising an amino acid sequence of SEQ ID NOS: 199 or 200 (NKp46-1 H1 or H3 variable domain), and a VL comprising an amino acid sequence of the amino acid sequence of SEQ ID NO: 201 (NKp46-1 L1 variable domain);
(b) a VH comprising an amino acid sequence of SEQ ID NOS: 202, 203 or 204 (NKp46-2 H1, H2 or H3 variable domain), and a VL comprising an amino acid sequence of SEQ ID NO: 205 (NKp46-2 L1 variable domain);
(c) a VH comprising an amino acid sequence of SEQ ID NOS: 206, 207 or 208 (NKp46-3 H1, H3 or H4 variable domain), and a VL comprising an amino acid sequence of SEQ ID NO: 209 (NKp46-3 L1 variable domain);
(d) a VH comprising an amino acid sequence of SEQ ID NOS: 210, 211 or 212 (NKp46-4 H1 variable domain), and a VL comprising an amino acid sequence of SEQ ID NO: 213 (NKp46-4 L2 variable domain);
(e) a VH comprising an amino acid sequence of SEQ ID NO: 215 (NKp46-9 H2 variable domain), and a VL comprising an amino acid sequence of SEQ ID NOS: 217 or 218 (NKp46-9 L1 or L2 variable domain); or
(f) a VH comprising an amino acid sequence of SEQ ID NO: 216 (NKp46-9 H3 variable domain), and a VL comprising an amino acid sequence of SEQ ID NOS: 217 or 218 (NKp46-9 L1 or L2 variable domain).

2. A multispecific antigen binding protein, a protein, or polypeptide comprising an antigen binding domain that binds a human NKp46 polypeptide, wherein the domain comprises a heavy chain variable region (VH) and a light chain variable region (VL) combination selected from the group consisting of:
(a) a VH comprising an amino acid sequence of SEQ ID NOS: 199 or 200 (NKp46-1 H1 or H3 variable domain), and a VL comprising an amino acid sequence of the amino acid sequence of SEQ ID NO: 201 (NKp46-1 L1 variable domain);
(b) a VH comprising an amino acid sequence of SEQ ID NOS: 202, 203 or 204 (NKp46-2 H1, H2 or H3 variable domain), and a VL comprising an amino acid sequence of SEQ ID NO: 205 (NKp46-2 L1 variable domain);
(c) a VH comprising an amino acid sequence of SEQ ID NOS: 206, 207 or 208 (NKp46-3 H1, H3 or H4 variable domain), and a VL comprising an amino acid sequence of SEQ ID NO: 209 (NKp46-3 L1 variable domain);
(d) a VH comprising an amino acid sequence of SEQ ID NOS: 210, 211 or 212 (NKp46-4 H1 variable domain), and a VL comprising an amino acid sequence of SEQ ID NO: 213 (NKp46-4 L2 variable domain);
(e) a VH comprising an amino acid sequence of SEQ ID NO: 215 (NKp46-9 H2 variable domain), and a VL comprising an amino acid sequence of SEQ ID NOS: 217 or 218 (NKp46-9 L1 or L2 variable domain); or
(f) a VH comprising an amino acid sequence of SEQ ID NO: 216 (NKp46-9 H3 variable domain), and a VL comprising an amino acid sequence of SEQ ID NOS: 217 or 218 (NKp46-9 L1 or L2 variable domain).

3. The multispecific antigen binding protein, protein, or polypeptide of claim 2, wherein the protein is a multispecific protein further comprising a second antigen binding domain that binds to an antigen of interest, wherein the multispecific protein is capable of directing an NKp46-expressing NK cell to lyse a target cell expressing the antigen of interest.

4. The multispecific antigen binding protein, protein, or polypeptide of claim 3, wherein the protein is a heterodimer and comprises:
(a) a first polypeptide having a domain arrangement:
Vₐ₋₁-(CH1 or CK)ₐ-Fc domain-Vₐ₋₂-V_{b-2},
and
(b) a second polypeptide chain having a domain arrangement:
V_{b-1}-(CH1 or CK)-Fc domain
wherein one of Vₐ₋₁ and V_{b-1} is a light chain variable domain and the other is a heavy chain variable domain, one of Vₐ₋₂ and V_{b-2} is a light chain variable domain and the other is a heavy chain variable domain;
wherein (CH1 or CK)_{b} dimerizes with the (CH1 or CK)ₐ on the central chain, and the V_{b-1} forms an antigen binding domain together with Vₐ₋₁ of the central chain, and wherein Vₐ₋₂ and V_{b-2} together form an antigen binding domain.

5. The multispecific antigen binding protein, protein, or polypeptide of claim 3, wherein the protein is a heterotrimer and comprises:
(a) a first polypeptide chain having a domain arrangement:
Vₐ₋₁-(CH1 or CK)ₐ-Fc domain-Vₐ₋₂-(CH1 or CK)_{b},
(b) a second polypeptide chain having a domain arrangement:
V_{b-1}-(CH1 or CK)_{c}-Fc domain,
and
(c) a third polypeptide chain having a domain arrangement:
V_{b-2}-(CH1 or CK)_{d},
wherein one of Vₐ₋₁ and V_{b-1} is a light chain variable domain and the other is a heavy chain variable domain, one of Vₐ₋₂ and V_{b-2} is a light chain variable domain and the other is a heavy chain variable domain;
wherein (CH1 or CK)_{c} dimerizes with the (CH1 or CK)ₐ on the central chain, and the Vₐ₋₁ and V_{b-1} form an antigen binding domain; and
wherein (CH1 or CK)_{d} dimerizes with the (CH1 or CK)_{b} unit on the central chain, and the Vₐ₋₂ and V_{b-2} form an antigen binding domain.

6. An isolated nucleic acid encoding a multispecific antigen binding protein, protein, or polypeptide of claim 2, or a polypeptide chain thereof.

7. A recombinant host cell comprising a nucleic acid according to claim 6.

8. A method of producing a protein comprising culturing a cell of claim 7 under conditions suitable for expression of the protein and recovering the protein.

9. A method of treating a disease in a subject comprising administering to the subject a multispecific antigen binding protein, protein, or polypeptide of claim 2.
